SEQUENCE LISTING (56 sequences)

SEQ ID NO: 1 - DNA, 2409 bp, synthetic construct
  source 1..2409: mol_type = other DNA
gtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaa

SEQ ID NO: 2 - DNA, 2409 bp, synthetic construct
  source 1..2409: mol_type = other DNA
gtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggatggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaa

SEQ ID NO: 3 - protein, 802 aa, synthetic construct
VLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 4 - DNA, 5616 bp, synthetic construct
  source 1..5616: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggtctagagctagctgtcgtgaggaatttcgacatttaaatttaattaagccaccatggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggatggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaattaattaatctcgacggtatcggttaacttttaaaagaaaagggggaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgaaatcggatccagctaaatctaagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggggagggcagaggaagtcttctaacatgcggtgacgtggaggagaatcccggccctgaattcatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 5 - DNA, 11078 bp, synthetic construct
  source 1..11078: mol_type = other DNA
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 6 - DNA, 13504 bp, synthetic construct
  source 1..13504: mol_type = other DNA
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaagccaccatggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaattaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 7 - DNA, 43 bp, synthetic construct
  source 1..43: mol_type = other DNA
taagcattaattaagccaccatggtgctgctgtcccgcaagcg

SEQ ID NO: 8 - DNA, 34 bp, synthetic construct
  source 1..34: mol_type = other DNA
tgcttattaattaattacttgaaggcctccggaa

SEQ ID NO: 9 - DNA, 21 bp, synthetic construct
  source 1..21: mol_type = other DNA
tcaagcctcagacagtggttc

SEQ ID NO: 10 - DNA, 20 bp, synthetic construct
  source 1..20: mol_type = other DNA
cctcacattgccaaaagacg

SEQ ID NO: 11 - DNA, 35 bp, synthetic construct
  source 1..35: mol_type = other DNA
actaaagctagctgtcgtgaggaatttcgacattt

SEQ ID NO: 12 - DNA, 38 bp, synthetic construct
  source 1..38: mol_type = other DNA
actaaaaccggtcccccttttcttttaaaagttaaccg

SEQ ID NO: 13 - DNA, 20 bp, synthetic construct
  source 1..20: mol_type = other DNA
gtacggtgggcgcctataaa

SEQ ID NO: 14 - DNA, 22 bp, synthetic construct
  source 1..22: mol_type = other DNA
gggcaccgtctgaagcgttctt

SEQ ID NO: 15 - DNA, 22 bp, synthetic construct
  source 1..22: mol_type = other DNA
gcagtggactcagcagcacctg

SEQ ID NO: 16 - DNA, 22 bp, synthetic construct
  source 1..22: mol_type = other DNA
gcggcccatgttgtcctggatg

SEQ ID NO: 17 - DNA, 22 bp, synthetic construct
  source 1..22: mol_type = other DNA
gagggcatgctggaggacctca

SEQ ID NO: 18 - DNA, 22 bp, synthetic construct
  source 1..22: mol_type = other DNA
ggaagcaaggaggccaaggacc

SEQ ID NO: 19 - DNA, 22 bp, synthetic construct
  source 1..22: mol_type = other DNA
gtgaaattcagggcccctccgc

SEQ ID NO: 20 - DNA, 22 bp, synthetic construct
  source 1..22: mol_type = other DNA
ccctgcagcatggcatggtagg

SEQ ID NO: 21 - DNA, 22 bp, synthetic construct
  source 1..22: mol_type = other DNA
ggggctctcctggggcagaata

SEQ ID NO: 22 - DNA, 20 bp, synthetic construct
  source 1..20: mol_type = other DNA
gggtatcgacagagtgccag

SEQ ID NO: 23 - DNA, 21 bp, synthetic construct
  source 1..21: mol_type = other DNA
ctgagttcctgcgtacctatg

SEQ ID NO: 24 - DNA, 21 bp, synthetic construct
  source 1..21: mol_type = other DNA
tgccattctccatttgctttg

SEQ ID NO: 25 - DNA, 22 bp, synthetic construct
  source 1..22: mol_type = other DNA
ctcagatcattgtcacagtcgt

SEQ ID NO: 26 - DNA, 21 bp, synthetic construct
  source 1..21: mol_type = other DNA
gtcgtcaggaagaagagaacc

SEQ ID NO: 27 - DNA, 17 bp, synthetic construct
  source 1..17: mol_type = other DNA
acagagcctcgcctttg

SEQ ID NO: 28 - DNA, 17 bp, synthetic construct
  source 1..17: mol_type = other DNA
ccttgcacatgccggag

SEQ ID NO: 29 - DNA, 18 bp, synthetic construct
  source 1..18: mol_type = other DNA
catggcagacgagctgag

SEQ ID NO: 30 - DNA, 20 bp, synthetic construct
  source 1..20: mol_type = other DNA
aaactgtgtgtcccttctgg

SEQ ID NO: 31 - DNA, 20 bp, synthetic construct
  source 1..20: mol_type = other DNA
ctccaatcaacttgccagaa

SEQ ID NO: 32 - DNA, 20 bp, synthetic construct
  source 1..20: mol_type = other DNA
ataccagttggaggctggtc

SEQ ID NO: 33 - DNA, 23 bp, synthetic construct
  source 1..23: mol_type = other DNA
gaaccagatgcacattgatgaag

SEQ ID NO: 34 - DNA, 22 bp, synthetic construct
  source 1..22: mol_type = other DNA
tgcccacatattctcctttgag

SEQ ID NO: 35 - DNA, 19 bp, synthetic construct
  source 1..19: mol_type = other DNA
tgaccttctcctgccacta

SEQ ID NO: 36 - DNA, 21 bp, synthetic construct
  source 1..21: mol_type = other DNA
aagcgaaatcctcagtctgac

SEQ ID NO: 37 - DNA, 21 bp, synthetic construct
  source 1..21: mol_type = other DNA
cacttgaaaatgctccatgcc

SEQ ID NO: 38 - DNA, 20 bp, synthetic construct
  source 1..20: mol_type = other DNA
actcccgaaaaccaatgctc

SEQ ID NO: 39 - DNA, 22 bp, synthetic construct
  source 1..22: mol_type = other DNA
ttgttgtaggatatgcccttga

SEQ ID NO: 40 - DNA, 21 bp, synthetic construct
  source 1..21: mol_type = other DNA
gcgatgtcaataggactccag

SEQ ID NO: 41 - DNA, 20 bp, synthetic construct
  source 1..20: mol_type = other DNA
tggtgtcacagaggctacta

SEQ ID NO: 42 - DNA, 21 bp, synthetic construct
  source 1..21: mol_type = other DNA
gggctcgctcttctgattatt

SEQ ID NO: 43 - DNA, 19 bp, synthetic construct
  source 1..19: mol_type = other DNA
acatcgctcagacaccatg

SEQ ID NO: 44 - DNA, 22 bp, synthetic construct
  source 1..22: mol_type = other DNA
tgtagttgaggtcaatgaaggg

SEQ ID NO: 45 - DNA, 22 bp, synthetic construct
  source 1..22: mol_type = other DNA
tcgccgaccactaccagcagaa

SEQ ID NO: 46 - DNA, 22 bp, synthetic construct
  source 1..22: mol_type = other DNA
cgcgcttctcgttggggtcttt

SEQ ID NO: 47 - DNA, 22 bp, synthetic construct
  source 1..22: mol_type = other DNA
gcagtggactcagcagcacctg

SEQ ID NO: 48 - DNA, 22 bp, synthetic construct
  source 1..22: mol_type = other DNA
ctgcaggaggcgatcatgagcg

SEQ ID NO: 49 - DNA, 21 bp, synthetic construct
  source 1..21: mol_type = other DNA
atggcaaagtgaatgacaagc

SEQ ID NO: 50 - DNA, 21 bp, synthetic construct
  source 1..21: mol_type = other DNA
aggcgaagttaatggaagctc

SEQ ID NO: 51 - DNA, 19 bp, synthetic construct
  source 1..19: mol_type = other DNA
tggacacctgcatcaagac

SEQ ID NO: 52 - DNA, 22 bp, synthetic construct
  source 1..22: mol_type = other DNA
ggatcttcctcttgaactcctc

SEQ ID NO: 53 - DNA, 21 bp, synthetic construct
  source 1..21: mol_type = other DNA
gtgccatactcatgaccatcg

SEQ ID NO: 54 - DNA, 19 bp, synthetic construct
  source 1..19: mol_type = other DNA
ggccacaaagccaaagatg

SEQ ID NO: 55 - DNA, 21 bp, synthetic construct
  source 1..21: mol_type = other DNA
cgcgtctctccacatacattc

SEQ ID NO: 56 - DNA, 21 bp, synthetic construct
  source 1..21: mol_type = other DNA
gctggcttattctgagatgga

(19) United States
(12) Patent Application Publication
(10) Pub. No.: US 2025/0092369 A1
(43) Pub. Date: Mar. 20, 2025

(54) METHOD FOR IMPROVING TRANSCYTOSIS PROPERTIES OF HUMAN BLOOD-BRAIN BARRIER MODEL

(71) Applicant: Wisconsin Alumni Research Foundation, Madison, WI (US)

(72) Inventors: Eric Shusta, Madison, WI (US); Sarah Boutom, Madison, WI (US); Yunfeng Ding, Wellesley, MA (US); Benjamin Gastfriend, San Diego, CA (US); Sean Palecek, Verona, WI (US)

(21) Appl. No.: 18/889,235

(22) Filed: Sep. 18, 2024

Related U.S. Application Data
(60) Provisional application No. 63/583,449, filed on Sep. 18, 2023.

(51) Int. Cl. C12N 5/071; G01N 33/50

(52) U.S. Cl. CPC C12N 5/069; G01N 33/5064; C12N 2501/115; C12N 2501/415; C12N 2501/42; C12N 2506/45; C12N 2510/00

(57) ABSTRACT

The present invention provides cells and methods for producing endothelial cells with blood-brain barrier (BBB)-like transcytosis properties. Endothelial cells produced by the methods, BBB models comprising these endothelial cells, and methods of using the BBB models to test the ability of a therapeutic agent to cross the BBB are also provided.

### CROSS-REFERENCE TO RELATED APPLICATIONS

This application claims priority to U.S. Provisional Application No. 63/583,449 filed on Sep. 18, 2023, the contents of which are incorporated by reference in their entireties.

### STATEMENT REGARDING FEDERALLY SPONSORED RESEARCH

This invention was made with government support under NS107461 awarded by the National Institutes of Health. The government has certain rights in the invention.

### SEQUENCE LISTING

This application includes a sequence listing in XML format titled “960296_04543_ST26.xml”, which is 86,119 bytes in size and was created on Sep. 17, 2024. The sequence listing is electronically submitted with this application via Patent Center and is incorporated herein by reference in its entirety.

### BACKGROUND

The blood-brain barrier (BBB) is composed of specialized vascular endothelial cells that maintain brain homeostasis and regulate the passage of blood solutes into the central nervous system (CNS) by restricting both transcellular and paracellular transport. In vitro human pluripotent stem cell (hPSC)-derived BBB models have proven useful for the study of barrier regulation, molecular transport, and brain drug delivery. Such models offer improved recapitulation of human development and disease as compared to animal models, and offer greater accessibility as compared to primary human tissues. However, the brain microvascular endothelial cells (BMECs) that form the BBB in vivo exhibit reduced expression of vesicular transcytosis proteins relative to non-BBB endothelial cells, and this is not reflected in existing BBB models. As a result, existing BBB models do not accurately model trans-BBB transport of substances that utilize the caveolae-mediated transport pathway. Accordingly, there remains a need in the art for BBB models that have in vivo-like BBB transcytosis properties.

### SUMMARY

In a first aspect, the present invention provides gene-edited pluripotent stem cells that comprise a transgene comprising a polynucleotide encoding a Notch1 receptor intracellular domain (N1ICD) operably linked to an inducible promoter.

In a second aspect, the present invention provides endothelial progenitor cells differentiated from the pluripotent stem cells described herein.

In a third aspect, the present invention provides methods for producing an endothelial cell with BBB-like transcytosis properties. The methods comprise (a) culturing a CD34+CD31+ endothelial progenitor cell in a medium comprising a Wnt/β-catenin signaling activator; and (b) inducing Notch signaling after 2-7 days of culturing.

In fourth aspect, the present invention provides populations of endothelial cells with BBB-like transcytosis properties that are produced by the methods described herein.

In a fifth aspect, the present invention provides in vitro BBB models comprising a confluent monolayer of the endothelial cells described herein cultured on a surface.

In a sixth aspect, the present invention provides methods for using the BBB models described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows immunostaining of unedited human pluripotent stem cell-derived endothelial progenitor cells (hPSC-EPCs) with lentiviral-induced constitutive Notch1 intracellular domain (N1ICD) overexpression. Immunocytochemistry (ICC) analysis of various markers in hPSC-EPCs treated with dimethyl sulfoxide (DMSO), CHIR99021 (CHIR), CHIR and lentivirus encoding IRES-GFP (GFP LV), or CHIR and lentivirus encoding N1ICD-IRES-GFP (N1ICD LV) on D11 after 6 days of culture in hECSR medium supplemented with DMSO or CHIR and 4 days following transduction with either GFP LV or N1ICD LV. (A) Representative images of ICC analysis for caveolin-1, CD31, and GFP in various treatment conditions. Only transduced cells were stained for GFP. Hoechst nuclear counterstain is overlaid in all images. Scale bar: 100 μm. (B) Quantification of caveolin-1 mean gray value (MGV) in different conditions from (A), normalized to Hoechst MGV. (C) Representative images of ICC analysis for plasmalemma vesicle associated protein (PLVAP) and CD31 in various treatment conditions. GFP staining not shown. Hoechst nuclear counterstain is overlaid in all images. Scale bar: 100 μm. (D) Quantification of PLVAP MGV in different conditions from (C), normalized to Hoechst MGV. (E) Representative images of ICC analysis for GLUT-1 and CD31 in various treatment conditions. GFP staining not shown. Hoechst nuclear counterstain is overlaid in all images. Scale bar: 100 μm. (F) Quantification of GLUT-1 MGV in different conditions from (C), normalized to Hoechst MGV. In (B), (D), and (F), points represent n=3 biological replicates from one differentiation of IMR90-4 iPSC-derived endothelial progenitor cells (EPCs). Horizontal bars indicate the mean. Hoechst-normalized relative fluorescence for each of the three markers was further normalized within each analysis such that the mean of the DMSO condition was equal to 1. Statistical analyses were performed on Hoechst-normalized data; P-values: One-way ANOVA followed by Tukey's HSD test.

FIG. 2 shows an analysis of transcriptional changes in blood-brain barrier (BBB)-related markers in unedited hPSC-EPCs with lentiviral-induced constitutive N1ICD overexpression. RT-qPCR analysis of various markers in D11 hPSC-ECs with induced barrier properties, treated with CHIR and GFP LV or CHIR and N1ICD LV. Relative expression (2-ΔΔCt normalized to GAPDH) is shown for the vesicular transcytosis-related genes CAV1 (A), PLVAP (B), and MFSD2A (C); for N1ICD (D), GFP (E), CDH5 (F), CDH2 (G), and FN1 (H); for the tight junction-related genes CLDN5 (I), OCLN (J), and TJP1 (K); and for the efflux transport-related genes ABCB1 (L), ABCC1 (M), ABCG2 (N), and SLC2A1 (O), which encodes GLUT-1. In all analyses, points represent n=3 biological replicates from one differentiation of IMR90-4 iPSC-derived EPCs. Bars indicate mean±SD. Relative gene expression values normalized to GAPDH were further normalized within each analysis such that the mean of the CHIR+GFP LV condition was equal to 1. Statistical analyses were performed on normalized data; P-values<0.05 by Student's t-test are highlighted with an asterisk.

FIG. 3 shows differentiation and validation of gene-edited hPSC-EPCs comprising a doxycycline-inducible, N1ICD-overexpressing transposon plasmid (PB-TRE-N1ICD). (A) Schematic of the protocol for differentiation of human pluripotent stem cells (hPSCs) to endothelial progenitor cells (EPCs), followed by magnetic-activated cell sorting (MACS) sorting, hygromycin reselection and doxycycline treatment to overexpress N1ICD. (B) Schematic of the doxycycline-inducible, N1ICD-overexpressing transposon construct. The N1ICD cassette follows a TRE3G doxycycline inducible promoter. A cassette encoding the Tet-On 3G protein follows an EF-1α core promoter, resulting in constitutive expression of this protein. Tet-On 3G must associate with doxycycline to bind to the TRE3G inducible promoter. Also following the EF-1α promoter and Tet-On 3G are a self-cleaving 2A peptide linker (T2A) followed by a hygromycin resistance gene (HygR). The sequence of this construct is provided as SEQ ID NO: 4. The top portion of the schematic indicates that during differentiation of hPSCs to EPCs, the TRE3G promoter is not active due to the absence of doxycycline. The bottom portion indicates that hPSC-EPCs that have been differentiated and sorted can be treated with doxycycline to activate N1ICD overexpression. (C) Representative flow cytometry plots of D5 hPSC-EPCs derived from a population of gene-edited IMR90-4 iPSCs with heterogeneous copy numbers of the integrated construct shown in (B). Graphs show percentage of CD31+/CD34+ EPCs before (pre-MACS) and after (post-MACS) sorting. (D) Western blotting analysis of D15 hPSC-derived PB-TRE-N1ICD EPCs treated with CHIR, with or without doxycycline. Membranes were blotted for full-length Notch1 (N1 FL) and intracellular domain (N1ICD), as well as β-actin. Predicted approximate molecular weights of each detected protein are indicated on the right-hand side.

FIG. 4 shows immunostaining of gene-edited PB-TRE-N1ICD hPSC-EPCs+CHIR±Dox. Immunocytochemistry (ICC) analysis of various markers in gene-edited PB-TRE-N1ICD hPSC-EPCs on D15 after 10 days of culture in hECSR supplemented with CHIR and 1:1000 diluted DPBS from D5 to D11 and 4 days of CHIR and doxycycline (1 μg/mL) or PBS treatment from D11 to D15. (A) Representative images of ICC analysis for caveolin-1 and CD31. Hoechst nuclear counterstain overlaid in all images. Scale bar: 200 μm. (B) Quantification of caveolin-1 mean gray value (MGV) in different conditions from (A), normalized to Hoechst MGV. (C) Representative images of ICC analysis for PLVAP and CD31. Hoechst nuclear counterstain overlaid in all images. Scale bar: 200 μm. (D) Quantification of PLVAP MGV in different conditions from (C), normalized to Hoechst MGV. (E) Representative images of ICC analysis for GLUT-1 and CD31. Hoechst nuclear counterstain overlaid in all images. Scale bar: 200 μm. (F) Quantification of GLUT-1 mean gray value MGV in different conditions from (C), normalized to Hoechst MGV. In (B), (D), and (F), points represent n=4 biological replicates from one differentiation of IMR90-4 PB-TRE-N1ICD 10(+CR2) hPSC-derived EPCs. Horizontal bars indicate mean±SD. Hoechst-normalized relative fluorescence for each of the three markers was further normalized within each analysis such that the mean of the PBS condition was equal to 1. Statistical analyses were performed on Hoechst-normalized data; P-values: Student's t test.

FIG. 5 shows FACS sorting and bulk RNA sequencing of gene-edited PB-TRE-N1ICD 10(+CR2) hPSC-EPCs with doxycycline-inducible N1ICD overexpression. (A) Schematic of culture timeline and FACS sorting. PBS-treated (n=4) and Dox-treated (n=3) cells were sorted by FACS into subpopulations based on CD144 (VE-cadherin) expression. Lower boundary for CD144^{high} gate was set based on majority of PBS-treated gene-edited hPSC-EPCs and unedited hPSC-EPCs cultured in the presence of CHIR alone. Lower boundary for CD144^{low} gate was set based on CD144 antibody-stained undifferentiated hPSCs and isotype control antibody-stained edited hPSC-EPCs. Gated plots show representative distributions of PBS- and Dox-treated edited hPSC-EPCs into CD144^{high} and CD144^{low} expressing subpopulations. (B) Principal component analysis (PCA) plot showing relative differences in transcriptomic profile of bulk RNA-sequenced subpopulations from (A), including PBS-treated CD144^{high} (PBS), Dox-treated CD144^{high} (Doxhi), and Dox-treated CD144^{low} (Doxlo). Raw bulk RNA sequencing data were pre-processed using the Galaxy pipeline (usegalaxy.org) and analyzed in RStudio using DESeq2. (C) Normalized expression (transcripts per million, TPM) of NOTCH1 and HEYL from each bulk RNA-sequenced subpopulation discussed in (A) and (B). (D) Heatmap of row-normalized TPM expression for Notch signaling-related genes that are significantly differentially expressed (FDR<0.05) between the 3 subpopulations. Selected genes were obtained from KEGG and Hallmark Notch signaling gene sets via MSigDB (www.gsea-msigdb.org/gsea/msigdb). Row z-scores were calculated for each gene by subtracting mean expression across all samples from normalized expression for a specific sample and dividing the result by the standard deviation. Row z-scores range from bright red, indicating high expression, and dark blue, indicating low expression. Heatmap was produced in RStudio using the pheatmap package.

FIG. 6 shows a transcriptomic comparison of gene-edited PB-TRE-N1ICD hPSC-EPCs+CHIR±Dox to other cell types. (A) Analysis of bulk transcriptomic data of subpopulations of gene-edited PB-TRE-N1ICD 10(+CR2) hPSC-EPC subpopulations using PACNet⁴⁵ classification scores to assess similarity to generic cell types in a training data set. Sample replicates from left to right: PBS-treated, CD144-high (PBS_CD144hi); doxycycline-treated, CD144-high (Dox_CD144hi); doxycycline-treated, CD144-low (Dox_CD14410); random cell type (rand) auto-generated by PACNet software. Classification score colors ranging from black to yellow indicate no transcriptomic resemblance to 100% transcriptomic match, respectively, to training data cell types indicated on the y-axis. (B) PACNet analysis of primary human brain microvascular endothelial cells (HBMEC), immortalized hCMEC.D3 brain endothelial cells (hCMEC_D3), and primary human aortic endothelial cells (Haortic) relative to training set cell types. All analyses in (A) and (B) were performed using the PACNet webtool (cahanlab.org/resources/agnosticCellNet_web/). (C) PCA plot combining several in vivo and in vitro transcriptomic datasets, including: pseudo-bulked in vivo adult brain capillary endothelial cell single-cell RNA sequencing (scRNA-seq) data (Yang et al, 2022)⁴⁷; pseudo-bulked in vivo embryonic brain capillary endothelial cells scRNA-seq data (Crouch et al, 2022)⁴⁶; in vitro primary/immortalized brain endothelial and primary peripheral endothelial cells (similar to those presented in [B]) bulk RNA-seq data (Qian et al, 2017)⁵⁴; in vitro passage 1 hPSC-derived endothelial progenitor cells (EPCs) treated with CHIR and hPSC-derived smooth muscle-like cells (SMLCs) bulk RNA-seq data (Gastfriend et al, 2021)⁸; in vitro primary cells representing different developmental lineages bulk RNA-seq data (Pandey, 2022, GSE190615 [www.ncbi.nlm.nih.gov/geo/query/acc.cgi?acc=GSE190615]); in vitro gene-edited PB-TRE-N1ICD hPSC-derived endothelial progenitor cells treated with CHIR with or without doxycycline bulk RNA-seq data (this study). PCA plot was generated using the DESeq2 package in R.]

FIG. 7 shows normalized expression of selected BBB- and cell identity-related genes from bulk RNA-seq of edited PB-TRE-N1ICD 10(+CR2) hPSC-EPCs+CHIR±Dox subpopulations. Comparison of normalized gene expression (TPM) between bulk RNA-sequenced PBS- and Dox-treated subpopulations for NOTCH1 (A), CAV1 (B), PLVAP (C), MFSD2A (D), SLC2A1 (E), CLDN5 (F), OCLN (G), TJP1 (H), ABCB1 (I), ABCG2 (J), ABCC1 (K), PECAM1 (L), CDH5 (M), EPCAM (N), ACTA2 (O), and FN1 (P). In all analyses, points represent n=3 or n=4 biological replicates depending on treatment condition from one differentiation of gene-edited IMR90-4 PB-TRE-N1ICD 10(+CR2) hPSC-EPCs. Bars indicate mean±SD. Relative gene expression values are shown in TPM, and statistical analyses were performed on TPM data; P-values<0.05 by one-way ANOVA with post-hoc Tukey's test are highlighted using an asterisk.

FIG. 8 demonstrates that N1ICD overexpression in gene-edited PB-TRE-N1ICD hPSC-EPCs reduces fluorescent albumin accumulation by inhibiting caveolae-mediated endocytosis. (A) Representative live cell epifluorescence microscopy images of 10(+CR2) PB-TRE-N1ICD hPSC-EPCs cultured in hECSR+CHIR, with PBS or Dox, and PBS-cultured cells with methyl-β-cyclodextrin (MβCD, inhibitor of caveolae-mediated endocytosis) pretreatment that were incubated at 37° C. with bovine serum albumin (BSA) conjugated to AlexaFluor647 (albumin-AF647). CD144 (VE-cadherin) and AlexaFluor 647 are shown in the 488 and 647 nm channels, respectively. Hoechst nuclear counterstain is overlaid in all images. Scale bar: 200 μm. (B) Representative flow cytometry plots for isotype control, PBS- and Dox-treated cells from the fluorescent albumin accumulation assay showing gating of the CD144^{high} (CD144hi) and CD144^{low} (CD144lo) subpopulations. (C) Quantification of albumin-AF647 accumulation in gated subpopulations incubated at 37° C., both with and without MβCD pretreatment. Selected statistical comparisons shown between accumulation for each subpopulation and its respective MβCD-pretreated condition as well as between individual subpopulations in the absence of MβCD pretreatment. (D) Quantification of albumin-AF647 accumulation in all CD144-positive cells from each condition, with or without MβCD pretreatment. Conditions incubated at 37° C. as well as 4° C. control were included. Selected statistical comparisons are shown among PBS- or Dox-treated conditions in the presence and absence of MβCD-pretreatment. Statistical analyses for (C) and (D) were performed on raw MFI data; P-values<0.05 by one-way ANOVA with post-hoc Tukey's test.

FIG. 9 demonstrates that N1ICD overexpression in gene-edited PB-TRE-N1ICD 10(+CR2) hPSC-EPCs+CHIR reduces frequency of total and caveolae-associated vesicles. Transmission electron microscopy (TEM) analysis of PBS- or Dox-treated PB-TRE-N1ICD 10(+CR2) hPSC-EPCs. (A) Representative ultrastructural TEM images. Arrowheads indicate locations of vesicles. Scale bar: 400 nm. (B) Quantification of vesicle density relative to total cell area for each image. (C) Quantification of vesicle count per diameter range across all 12 images for PBS- or Dox-treated conditions. (D) Representative immunolabeled TEM image of Nanogold particles bound to caveolin-1 in PBS-treated cells. Scale bar: 100 nm. (E) Quantification of area fraction occupied by Nanogold particles relative to total cell area. (F) Quantification of caveolin-1/Nanogold-associated vesicles relative to total cell area. For (C), (E), and (F), points represent 3 images measured per biological replicate for n=4 replicates in each treatment condition. Horizontal bars indicate mean±SD. Statistical analyses were performed on cell area-normalized counts for (C) and (F) and on area fraction for (E). All P-values<0.05 by Student's t-test.

FIG. 10 shows the transfer plasmids for GFP and N1ICD lentiviruses. (A) Plasmid map for pWPI (Addgene #12254). The EF-1α core promoter is followed by an internal ribosome entry site (IRES) and GFP gene. (B) Plasmid map for pWPI-N1ICD (Addgene #185525). Identical to pWPI except that the EF-1α promoter is followed by a gene encoding human native Notch1 receptor intracellular domain (N1ICD), which is then followed by an IRES and GFP. Both plasmids also contain an ampicillin resistance gene (AmpR) downstream or the aforementioned cassettes for plasmid cloning and colony selection in E. coli.

FIG. 11 shows the protocol for hPSC-EPC differentiation and validation of lentiviral transduction. (A) Schematic of the protocol for differentiation of unedited hPSCs to EPCs⁴⁰. Once hPSC-EPCs have been differentiated and sorted, they can be cultured in hECSR medium (hESFM+B27+FGF2), optionally supplemented with small molecules, recombinant proteins, or genetic overexpression vehicles like lentivirus. In the case of our initial experiments, unedited hPSC-EPCs that had been cryopreserved after sorting were thawed and plated on D5, cultured with hECSR supplemented with CHIR until D7 when they were transduced with N1ICD LV or GFP LV and subsequently cultured for an additional 4 days. On D11, cells with high GLUT-1 expression and low expression of caveolin-1 and PLVAP were isolated for further characterization in downstream assays. (B) Schematic of the lentiviral transfer plasmid constructs for GFP LV and N1ICD LV. The sequence of the pWPI-IRES-GFP plasmid, which comprises the GFP LV construct, is provided as SEQ ID NO: 5, and the sequence of the pWPI-N1ICD-IRES-GFP, which comprises the N1ICD LV construct, is provided as SEQ ID NO: 6. EF-1α core promoter is followed by an IRES and GFP for both transfer plasmids. In N1ICD LV, IRES and GFP are preceded by a human native N1ICD cassette. Representative flow cytometry plots showing GFP and CD31 fluorescence in unedited hPSC-EPCs transduced with GFP LV (C) or N1ICD LV (D). Each analysis used an isotype control and un-transduced control to establish gates for both markers. CD31 mean fluorescence intensity (MFI) values are shown in underlined text for GFP negative cells. For GFP positive cells, CD31 MFI and GFP MFI values are shown in italic text and bold text, respectively. (E-G) Western blotting analysis of D11 hPSC-ECs with induced barrier properties, treated with CHIR and GFP LV or CHIR and N1ICD LV. (E) Membrane blotted for full-length Notch1 (N1 FL) and intracellular domain (N1ICD), followed by re-probing for β-actin. Predicted approximate molecular weights of each detected protein are indicated on the right-hand side. Quantification of western blot band intensities for N1 FL (F) and N1ICD (G), normalized to respective input control (β-actin) band intensities. In all western blot analyses, points represent n=4 biological replicates from one differentiation of IMR90-4 iPSC-derived EPCs. Bars indicate mean±SD. β-actin-normalized band intensities were further normalized within each analysis such that the mean of the CHIR+GFP LV condition was equal to 1. Statistical analyses were performed on β-actin-normalized data; P-values: Student's t-test.

FIG. 12 shows caveolin-1 flow cytometry in transduced unedited hPSC-EPCs and human umbilical vein endothelial cells (HUVECs). (A) Representative flow cytometry plots for hPSC-EPCs cultured for 6 days (D5-D11) in hECSR supplemented with CHIR and transduced on D7 with GFP LV or N1ICD LV. Samples included a GFP single stain control (cells transduced with GFP LV, not stained with caveolin-1-PE) and caveolin-1 single stain control (un-transduced cells, stained with caveolin-1-PE; data not shown). (B) Representative flow cytometry plots for HUVECs transduced with GFP LV or N1ICD LV and cultured for 6 days in EGM-2 medium. Samples included similar controls as described in (A). (C) Quantification of caveolin-1 expression (MFI) in hPSC-EPCs treated with CHIR and GFP LV versus in hPSC-EPCs treated with CHIR and N1ICD LV. Points represent n=3 biological replicates from one differentiation of IMR90-4 iPSC-derived EPCs. Bars indicate mean±SD. (D) Quantification of caveolin-1 expression (MFI) in HUVECs transduced with GFP LV versus in HUVECs transduced with N1ICD LV. Points represent n=3 biological replicates. Bars indicate mean±SD. Statistical analyses were performed on un-normalized data; P-values: Student's t-test.

FIG. 13 shows CD31 flow cytometry in transduced hPSC-EPCs and HUVECs. (A) Representative flow cytometry plots for hPSC-EPCs cultured for 6 days (D5-D11) in hECSR supplemented with CHIR and transduced on D7 with GFP LV or N1ICD LV. Samples included a GFP single stain control (cells transduced with GFP LV, not stained with CD31-APC) and CD31 single stain control (un-transduced cells, stained with CD31-APC; data not shown). (B) Representative flow cytometry plots for HUVECs transduced with GFP LV or N1ICD LV and cultured for 6 days in EGM-2 medium. Samples included similar controls as described in (A). (C) Quantification of CD31 expression (MFI) in hPSC-EPCs treated with CHIR and GFP LV versus in hPSC-EPCs treated with CHIR and N1ICD LV. Points represent n=3 biological replicates from one differentiation of IMR90-4 iPSC-derived EPCs. Bars indicate mean±SD. (D) Quantification of CD31 expression (MFI) in HUVECs transduced with GFP LV versus in HUVECs transduced with N1ICD LV. Points represent n=3 biological replicates. Bars indicate mean±SD. Statistical analyses were performed on un-normalized data; P-values: Student's t-test.

FIG. 14 shows the generation of a gene-edited hPSC line with doxycycline-inducible overexpression of N1ICD. (A) Schematic of the PB-TRE-N1ICD plasmid, which contains a transposon encoding N1ICD overexpression elements that will be integrated into the hPSC genome by a piggyBac transposase. The transposon is located between 3′ and 5′ piggyBac inverted repeats. A TRE3G doxycycline-inducible promoter is followed by the human native N1ICD sequence (SEQ ID NO: 2), which is similar to the N1ICD sequence used in the pWPI-N1ICD plasmid (SEQ ID NO: 1) except that there is a silent mutation at amino acid 433 that was unintentionally introduced during cloning. There is also an EF-1α core promoter followed by a Tet-On 3G gene, followed by a hygromycin resistance cassette (HygR). Outside of the piggyBac inverted repeats is an ampicillin resistance gene that can be used for selection of transformed E. coli clones during plasmid amplification. (B) Screenshot of Sanger sequencing results for the cloned PB-TRE-N1ICD plasmid, which show a silent mutation (GAC→GAT) in coding sequence of N1ICD for amino acid 433 (aspartic acid). Note: The top DNA sequence is a portion of SEQ ID NO: 1 comprising residues 1,277-1,310, the bottom DNA sequence is the corresponding portion of SEQ ID NO: 2 (residues 1,277-1,310), and the amino acid sequence is the corresponding part of SEQ ID NO: 3 (residues 426-437). (C) Gel electrophoresis image showing purified PB-TRE-N1ICD from 3 separate clones (c4, c5, c6), digested at both NheI and AgeI restriction sites. Ladder band sizes are shown on the right. All 3 clones have bands of the expected size for the doxycycline-inducible plasmid backbone (˜8 kb) and N1ICD insert (˜2.5 kb). A band of ˜10.5 kb, corresponding to a small fraction of plasmid digested at a single restriction site, is also seen. Clone c6 was Sanger sequenced (results shown in (A) and (B). (D-E) RT-qPCR analysis of a population of gene-edited IMR90-4 iPSCs containing heterogeneous copy numbers of the PB-TRE-N1ICD transposon, after 3 days treatment with 1 μg/mL doxycycline or PBS control. Expression of NOTCH1 (D) and HEYL (E) (a gene downstream of Notch signaling activation) are expressed as 2^{-ΔΔCt} normalized to GAPDH. Points represent n=3 biological replicates. Horizontal bar indicates mean. Relative gene expression values normalized to GAPDH were further normalized within each analysis such that the mean of the PBS condition was equal to 1. Statistical analyses were performed on GAPDH-normalized data; P-values: Student's t-test.

FIG. 15 shows the optimization of culture parameters for gene-edited PB-TRE-N1ICD hPSC-EPCs. Duration of hygromycin reselection and doxycycline treatment were initially optimized in a population of gene-edited IMR90-4 iPSCs containing heterogeneous copy numbers of the PB-TRE-N1ICD transposon. (A-B) Flow cytometry data analysis for D13, D15, or D17 hPSC-EPCs cultured in hECSR supplemented with CHIR, hygromycin, and doxycycline for various durations. A quantification of percentage CD31+ cells (A) and CD31 MFI (B) is shown for each condition. Points represent n=2 biological replicates from one differentiation of heterogenous gene-edited IMR90-4 iPSC-derived EPCs. Bars indicate mean±SD. (C) Immunocytochemistry (ICC) analysis of a D15 heterogeneous population edited hPSC-derived EPCs cultured in hECSR supplemented with CHIR and treated with 10 μg/mL hygromycin for 6 days followed by 1 μg/mL doxycycline (1:1000 diluted from stock) or PBS control. Representative images of ICC analysis for caveolin-1 and CD31 for doxycycline- or PBS-treated edited hPSC-EPCs. Hoechst nuclear counterstain is overlaid in all images. Scale bar: 100 μm. (D) Quantification of caveolin-1 and CD31 expression (MGV) in doxycycline- or PBS-treated cells from (B), normalized to Hoechst MGV. Points represent n=3 biological replicates from one differentiation of heterogenous gene-edited IMR90-4 iPSC-derived EPCs. Bars indicate mean±SD. Hoechst-normalized relative fluorescence for each marker was further normalized within each analysis such that the mean of the PBS condition was equal to 1. Statistical analyses were performed on Hoechst-normalized data; P-values: Student's t-test.

FIG. 16 shows a characterization of clonal gene-edited PB-TRE-N1ICD hPSC-EPCs. (A) RT-qPCR quantification of piggyBac transposon copy number in 11 clonal populations of gene-edited IMR90-4 iPSCs containing the PB-TRE-N1ICD transposon. The assay determines the number of piggyBac inserts relative to the 2 UCR1 loci per genome. PiggyBac insert copy number=0.5×(2^{-((average piggyBac insert Ct)-(average UCR1 Ct))}). Clone names are indicated on the x-axis; (+Y) or (+CR2) notation indicates that the edited IMR90-4 iPSC clones were generated in medium supplemented with the ROCK inhibitor Y-27632 or CloneR2, respectively. Clones with highly variable copy numbers are indicated by downward arrows and were selected for further characterization. (B) RT-qPCR analysis of N1ICD expression in PB-TRE-N1ICD hPSC clones 3(+Y) [48.3 copies], 9(+Y) [1.6 copies], 10(+CR2) [24.3 copies], and 11 (+CR2) [15.2 copies] treated for 3 days with various dilutions of doxycycline (1:1000, 1:5000, and 1:10,000, corresponding to 1 μg/mL, 200 ng/mL, and 100 ng/mL, respectively) or PBS control, with n=2 biological replicates per condition. An additional unedited hPSC sample (n=1) was included for reference. N1ICD expression is expressed as 2^{-ΔΔCt} normalized to HPRT1. Bars indicate mean±SD. Relative gene expression values normalized to HPRT1 were further normalized to unedited hPSCs such that relative N1ICD expression in unedited hPSCs was equal to 1.

FIG. 17 shows BBB gene expression in clonal gene-edited PB-TRE-N1ICD hPSC-EPCs+CHIR±different doxycycline concentrations. RT-qPCR analysis of various markers in D15 gene-edited clonal hPSC-EPCs treated with CHIR with or without various doses of doxycycline (100 ng/mL [1:10K], 200 ng/mL [1:5K], or 1 μg/mL [1:1K]) or 1:1000 diluted PBS. Relative expression (2^{-ΔΔCt} normalized to ACTB) of (A) NOTCH1, (B) MFSD2A, (C) CAV1, (D) PLVAP, (E) SLC2A1, and (F) CDH5 in EPCs derived from edited hPSC clones 3(+Y), 9(+Y), 10(+CR2), and 11 (+CR2). In all analyses, points represent n=3 biological replicates from one differentiation of IMR90-4 PB-TRE-N1ICD 10(+CR2) hPSC-derived EPCs. Bars indicate mean±SD. Relative gene expression values normalized to ACTB were further normalized within each analysis for (A)-(F) such that the mean of the 3(+Y) 1:1K PBS condition was equal to 1. Statistical analyses were performed on normalized data; P-values: One-way ANOVA with post-hoc Tukey's HSD test.

FIG. 18 shows immunostaining of gene-edited PB-TRE-N1ICD 10(+CR2) hPSC-EPCs with doxycycline-inducible N1ICD overexpression. Immunocytochemistry (ICC) analysis of BBB markers in gene-edited PB-TRE-N1ICD hPSC-EPCs treated with CHIR and 1:1000 diluted PBS or CHIR and 1 μg/mL doxycycline on D15 after 10 days of culture in hECSR supplemented with CHIR from D5 to D11 and 4 days of CHIR and doxycycline or PBS treatment from D11 to D15. (A) Representative images of ICC analysis for claudin-5 and CD31. Hoechst nuclear counterstain is overlaid in all images. Scale bar: 200 μm. (B) Quantification of claudin-5 mean gray value (MGV) in different conditions from (A), normalized to Hoechst MGV. (C) Representative images of ICC analysis for P-glycoprotein (P-gp) and CD31. Hoechst nuclear counterstain is overlaid in all images. Scale bar: 200 μm. (D) Quantification of P-gp MGV in different conditions from (C), normalized to Hoechst MGV. In (B) and (D), points represent n=4 biological replicates from one differentiation of IMR90-4 PB-TRE-N1ICD 10(+CR2) hPSC-derived EPCs. Horizontal bars indicate mean±SD. Hoechst-normalized relative fluorescence for each of the three markers was further normalized within each analysis such that the mean of the PBS condition was equal to 1. Statistical analyses were performed on Hoechst-normalized data; P-values: Student's t test.

FIG. 19 shows an analysis of transcriptional changes in BBB-related markers in gene-edited PB-TRE-N1ICD 10(+CR2) hPSC-EPCs with doxycycline-inducible N1ICD overexpression with or without hygromycin preconditioning and early or delayed doxycycline dosing. RT-qPCR analysis of various markers in D15 gene-edited PB-TRE-N1ICD 10(+CR2) hPSC-EPCs cultured in hECSR supplemented with CHIR with either 6 days of hygromycin followed by 4 days of DPBS (6d H+4d P); 6 days of hygromycin followed by 4 days of doxycycline (6d H+4d D); 6 days of DPBS followed by 4 days of doxycycline (6d P+4d D); or 2 days of DPBS, followed by 4 days of doxycycline, followed by 4 days of DPBS (2d P+4d D+4d P). (A-C) Relative expression of vesicular transcytosis-related genes CAV1 (A), PLVAP (B), and MFSD2A (C). (D-G) Relative expression for N1ICD (D), CDH5 (E), CDH2 (F), and FN1 (G). (H-J) Relative expression of tight junction-related genes CLDN5 (H), OCLN (I), and TJP1 (J). (K-N) Relative expression of efflux transport-related genes ABCB1 (K), ABCC1 (L), ABCG2 (M), and SLC2A1 (N). In all analyses, points represent n=3 biological replicates from one differentiation of gene-edited IMR90-4 PB-TRE-N1ICD 10(+CR2) hPSC-EPCs. Horizontal bars indicate mean±SD. Relative gene expression values (2^{-ΔΔCt} normalized to ACTB) were further normalized within each analysis such that the mean of the 6d H+4d P condition was equal to 1. Statistical analyses were performed on normalized data; P-values<0.05 by one-way ANOVA w/post-hoc Dunnett's test.

FIG. 20 shows an analysis of transcriptional changes in BBB-related markers in gene-edited PB-TRE-N1ICD 10(+CR2) hPSC-EPCs with doxycycline-inducible N1ICD overexpression. RT-qPCR analysis of various markers in D15 ECs with induced barrier properties derived from the 10(+CR2) gene-edited IMR90-4 iPSC clone with doxycycline-inducible N1ICD overexpression. D5 hPSC-EPCs derived from this edited clone were reselected for 6 days with hygromycin followed by 4 days of doxycycline treatment. (A-C) Relative expression (2^{-ΔΔCt} normalized to ACTB) of vesicular transcytosis-related genes CAV1 (A), PLVAP (B), and MFSD2A (C). (D-G) Relative expression (2^{-ΔΔCt} normalized to ACTB) for N1ICD (D), CDH5 (E), CDH2 (F), and FN1 (G). (H-J) Relative expression (2^{-ΔΔCt} normalized to ACTB) of tight junction-related genes CLDN5 (H), OCLN (I), and TJP1 (J). (K-N) Relative expression (2^{-ΔΔCt} normalized to ACTB) of efflux transport-related genes ABCB1 (K), ABCC1 (L), ABCG2 (M), and SLC2A1 (N). In all analyses, points represent n=3 biological replicates from one differentiation of IMR90-4 iPSC-derived EPCs. Bars indicate mean±SD. Relative gene expression values normalized to ACTB were further normalized within each analysis such that the mean of the condition treated with PBS was equal to 1. Statistical analyses were performed on normalized data; P-values<0.05 by Student's t-test are highlighted using an asterisk.

FIG. 21 shows differential expression of genes expected to be up- or down-regulated at the BBB in PB-TRE-N1ICD 10(+CR2) hPSC-EPCs+CHIR±Dox based on bulk RNA-seq. Endothelial cells were isolated using Seurat from an integrated single cell transcriptomic analysis²⁵ of multiple organs, including brain vasculature and peripheral organ vascular beds from lung, liver, heart and skeletal muscle. From this, 310 genes were identified to be significantly differentially expressed (absolute value [log (fold change)])>1, false discovery rate <0.05) between brain endothelial cells and peripheral organ endothelial cells. 173 genes were found to be upregulated and 137 were found to be downregulated in brain compared to peripheral endothelium. Heatmaps show relative expression of the 310 genes between the three subpopulations in the PBS- and doxycycline-treated gene-edited PB-TRE-N1ICD 10(+CR2) hPSC-EPCs (PBS-treated, CD144hi; Dox-treated, CD144hi; Dox-treated, CD144lo). (A) Of the 173 genes expected to be upregulated in brain endothelial cells, 46 are appropriately upregulated in response to N1ICD overexpression (highest in Dox_CD144lo subpopulation). 56/173 genes are downregulated in response to N1ICD overexpression, 61/173 are not differentially expressed, and 10 are not expressed (TPM=0). (B) Of the 137 genes expected to be downregulated in brain endothelial cells, 50 are appropriately downregulated in response to N1ICD overexpression (lowest in Dox_CD144lo subpopulation). 27/137 genes are upregulated in response to N1ICD overexpression, 59/137 are not differentially expressed, and 1 is not expressed (TPM=0). Heatmap columns indicate sample/replicate and rows indicate genes. Cell color indicates row z-score normalized gene expression level, ranging from blue (lowest expression) to red (highest). Heatmaps were generated using the pheatmap package in R. (C-D) MA plots showing log fold change in doxycycline-treated condition relative to PBS control versus log mean normalized counts of selected significantly differentially expressed BBB marker genes and BBB-enriched transcription factors and other transcriptional regulatory factors (TFORFs) seen in the comparison of the (C) Dox-treated CD144 high (Dox_CD144hi) vs. PBS_CD144hi or (D) Dox-treated CD144 low (Dox_CD144lo) subpopulation vs. PBS_CD144hi. For both plots, red dots indicate selected genes, blue dots indicate other significantly differentially expressed genes (FDR<0.05), and black dots indicate genes that are not significantly differentially expressed. MA plots were generated using ideal package in R.

FIG. 22 shows relative expression of selected BBB-related genes in PBS- and Dox-treated subpopulations of gene-edited PB-TRE-N1ICD 10(+CR2) hPSC-EPCs based on bulk RNA-seq. Average log₂ (fold change) in Dox-treated subpopulation (CD144hi or CD14410) relative to PBS control and average log₂ (TPM) for Dox-treated cells from CD144hi or CD144lo subpopulations of various genes related to BBB and general endothelial cell properties. Genes in each category were selected based on literature, including a list used in a recent publication by Porkoláb et al⁷³. The categories of genes listed include (A) transcytosis-related, (B) tight junction-related, (C) efflux transport-related, (D) adherens junction-related, (E) endothelial transcription factor-related, and (F) SLC cassette nutrient transporter-related. Values for average log₂ (fold change) in Dox-versus PBS-treated cells were obtained from DESeq2 analysis. Hatching of the bars indicates that the fold change is not statistically significant (FDR≥0.05). In graphs of normalized gene expression (average log₂[TPM]), error bars indicate standard deviation between replicates for Dox-treated cells. All graphs generated using GraphPad Prism.

FIG. 23 demonstrates that N1ICD overexpression in gene-edited PB-TRE-N1ICD 10(+CR2) hPSC-EPCs and primary endothelial cells reduces fluorescent albumin accumulation by inhibiting caveolae- and clathrin-mediated endocytic pathways. Quantification of flow cytometry-based measurement of endocytic uptake of bovine serum albumin (BSA) conjugated to AlexaFluor647 (albumin-AF647) into (A-F) gene-edited PB-TRE-N1ICD hPSC-EPCs treated with doxycycline or PBS or (G) human umbilical vein endothelial cells (HUVECs) treated with N1ICD overexpressing lentivirus or control GFP lentivirus. (A) Quantification of albumin-AF647 accumulation in gated subpopulations for 10(+CR2) hPSC-EPCs incubated at 37° C., both with and without chlorpromazine (CPZ, clathrin-mediated endocytosis inhibitor) or rottlerin (macropinocytosis inhibitor) pretreatment. Selected statistical comparisons between accumulation for each subpopulation and its respective CPZ- or rottlerin-pretreated condition as well as between individual subpopulations in the absence of CPZ or rottlerin pretreatment are shown. (B) Quantification of albumin-AF647 accumulation in all CD144-positive cells from each condition for 10(+CR2) hPSC-EPCs, with or without CPZ or rottlerin pretreatment. Conditions incubated at 37° C. as well as 4° C. control were included. Selected statistical comparisons are shown among PBS- or Dox-treated conditions in the presence and absence of CPZ or rottlerin pretreatment. (C) Quantification of albumin-AF647 accumulation in all CD144^{high} cells from each condition for 10(+CR2) hPSC-EPCs, with or without CPZ or rottlerin pretreatment. Conditions incubated at 37° C. as well as 4° C. control were included. Selected statistical comparisons are shown among PBS- or Dox-treated conditions in the presence and absence of CPZ or rottlerin pretreatment. (D) Quantification of albumin-AF647 accumulation in all CD144-positive cells from each condition for 3(+Y) hPSC-EPCs. Conditions incubated at 37° C. as well as 4° C. control were included. Selected statistical comparisons are shown among PBS- or Dox-treated conditions at both temperatures. (E) Quantification of albumin-AF647 accumulation in all CD144-positive cells from each condition for 9(+Y) hPSC-EPCs. Conditions incubated at 37° C. as well as 4° C. control were included. Selected statistical comparisons are shown among PBS- or Dox-treated conditions at both temperatures. (F) Quantification of albumin-AF647 accumulation in all CD144-positive cells from each condition for 11 (+CR2) hPSC-EPCs. Conditions incubated at 37° C. as well as 4° C. control were included. Selected statistical comparisons are shown among PBS- or Dox-treated conditions at both temperatures. (G) Quantification of albumin-AF647 accumulation in HUVECs transduced with N1ICD LV or GFP LV, with or without MβCD pretreatment. Conditions incubated at 37° C. as well as 4° C. control were included. Selected statistical comparisons are shown among GFP LV- or N1ICD LV-transduced conditions in the presence and absence of MβCD-pretreatment. Points in each graph represent n=3 biological replicates from one differentiation of gene-edited PB-TRE-N1ICD hPSCs or vial of HUVECs, respectively. Horizontal bars indicate mean±SD. Statistical analyses for all comparisons were performed on raw MFI data; P-values<0.05 by one-way ANOVA with post-hoc Tukey's test.

FIG. 24 shows transendothelial electrical resistance (TEER) measurement in gene-edited PB-TRE-N1ICD 10(+CR2) hPSC-EPCs+CHIR±Dox. hPSC-EPCs were cultured in hECSR supplemented with CHIR, treated with or without 1 g/mL doxycycline from D11-D15. X axis indicates the day in the differentiation process based on the schematic shown in FIG. 5A. Points represent n=4 biological replicates from one independent differentiation of gene-edited IMR90-4 PB-TRE-N1ICD hPSC-EPCs. Horizontal bars indicate standard deviation of means of TEER at each time point.

### DETAILED DESCRIPTION

The present inventors have developed methods for generating endothelial cells with BBB-like transcytosis properties. As is described in the Examples, the inventors generated these cells by treating human pluripotent stem cell-derived endothelial progenitor cells with the small molecule CHIR99021 to activate Wnt/β-catenin signaling and overexpressing the Notch1 intracellular domain (N1ICD) to active Notch signaling. They demonstrate that these endothelial cells express low levels of vesicular transcytosis proteins, similar to the levels observed in the human BBB in vivo. BBB models that comprise these endothelial cells should more accurately model trans-BBB transport of therapeutic agents, particularly those that utilize the caveolae-mediated transport pathway.

While the inventors have previously shown that Wnt/β-catenin signaling drives endothelial progenitor cells towards a more central nervous system-like phenotype (Gastfriend et al. (2021) Elife 10, e70992; WO2022072354), the additional effects of Notch signaling on the transcytosis properties of these cells were unknown. The endothelial progenitor cells that the inventors generated via activation of Wnt/β-catenin signaling alone exhibited upregulation of BBB markers like the glucose transporter GLUT-1 and downregulation of the vesicular transcytosis-associated protein plasmalemma vesicle associated protein (PLVAP), but they also exhibited upregulation of the transcytosis-associated protein caveolin-1. In contrast, the endothelial progenitor cells generated using the methods of the present invention exhibit downregulation of both caveolin-1 and PLVAP while maintaining upregulation of GLUT-1. Thus, BBB models comprising the EPCs described herein are expected to have improved BBB transcytosis properties as compared to previously described models.

### Pluripotent Stem Cells:

In a first aspect, the present invention provides gene-edited pluripotent stem cells that comprise a transgene comprising a polynucleotide encoding a Notch1 receptor intracellular domain (N1ICD) operably linked to an inducible promoter.

The term “pluripotent stem cell” (PSC) refers to a cell that has the ability to differentiate into cells of all three germ layers (i.e., ectoderm, endoderm, and mesoderm). Pluripotent stem cells include embryonic stem cells (ESCs) and induced pluripotent stem cells (iPSCs). iPSCs are stem cells that are produced by genetically reprogramming a multipotent or somatic cell back to a pluripotent state. This is accomplished through forced expression of pluripotency-associated factors (e.g., Oct3/4, Sox2, Nanog, Tbx3, and Klf4/5). In preferred embodiments, the pluripotent stem cells used with the present invention are human pluripotent stem cells (hPSCs).

The pluripotent stem cells of the present invention are “gene-edited,” meaning that their genomes comprise one or more copies of a transgene, i.e., a gene that was artificially introduced into a genome.

As is described in the Examples, the inventors used a PiggyBac transposon vector to randomly insert a N1ICD-encoding transgene into the genome of pluripotent stem cells, which generated cells comprising various copy numbers of the transgene inserted into random genomic loci. The inventors tested pluripotent stem cells that comprised about 2 to about 48 copies of the transgene. Thus, in some embodiments, the pluripotent stem cells comprise 2-48 copies of the transgene. In some embodiments, the pluripotent stem cells comprise about 2, about 15, about 24, or about 48 copies of the transgene. Those of skill in the art will appreciate that other means of inserting polynucleotide constructs into the genome could be used to introduce the transgene into pluripotent stem cells.

The pluripotent stem cells of the present invention comprise a transgene that encodes the intracellular domain of the transmembrane receptor Notch1. Notch signaling is activated by the binding of a Notch ligand to Notch1, which results in two sequential proteolytic cleavages that lead to the release of the Notch1 intracellular domain (N1ICD). The cleaved N1ICD subsequently translocates into the nucleus where it interacts with the transcription factor RBPJ, the coactivator MAML1, and the acetyltransferase EP300 to drive expression of Notch target genes. Thus, N1ICD is a fragment of the Notch1 receptor that functions as its activated form, and overexpression of N1ICD activates Notch signaling.

In the Examples, the inventors utilized two polynucleotide sequences that encode human N1ICD, namely SEQ ID NO: 1 and SEQ ID NO: 2. SEQ ID NO: 1, which is a DNA sequence that comprises nucleotides 5,522-7,930 of NCBI Reference Sequence: NM_017617.5, is the N1ICD-encoding sequence that the inventors used in their lentiviral transfer plasmid. SEQ ID NO: 2, which is identical to SEQ ID NO: 1 except that it comprises a silent mutation at nucleotide 1,296, is the N1ICD-encoding sequence the inventors used in their transposon plasmid. Thus, in some embodiments, the polynucleotide encoding N1ICD comprises or consists of SEQ ID NO: 1 or SEQ ID NO: 2. Both of these sequences encode the N1ICD protein of SEQ ID NO: 3. Thus, in some embodiments, the N1ICD comprises or consists of SEQ ID NO: 3. The N1ICD fragment may be expressed as a single, stand-alone protein by adding a methionine to its N-terminus to allow for translation. Alternatively, the N1ICD may be expressed as part of a fusion protein. In these embodiments, an enzyme cleavage site or self-cleaving peptide (e.g., a 2A peptide) may be included between the N1ICD and the other component(s) of the fusion protein to allow for its separation therefrom.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages).

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can drive transcription of the polynucleotide.

An “inducible promoter” is a promoter that only drives transcription under particular conditions or in the presence of a particular molecule (i.e., an “induction reagent”). Inducible promoters are commonly activated using fusion proteins comprising a sensor domain fused to a DNA binding domain (e.g., TALEs or dCas9) and a transcriptional activation domain (e.g., VP64, SunTag). After the sensor domain binds to its activator molecule, the fusion protein undergoes a conformational change that allows the DNA binding domain to bind to DNA and recruit the transcriptional activation to the inducible promoter. For a detailed description of such inducible systems, see Doshi et al. (Crit Rev Biotechnol 40(8): 1131-1150, 2020), which is hereby incorporated by reference in its entirety. In the Examples, the inventors utilized the doxycycline-inducible promoter TRE3G to drive N1ICD overexpression in their gene-edited pluripotent stem cells. Thus, in some embodiments, the inducible promoter is a doxycycline-inducible promoter (i.e., a promoter that is activated in the presence of doxycycline).

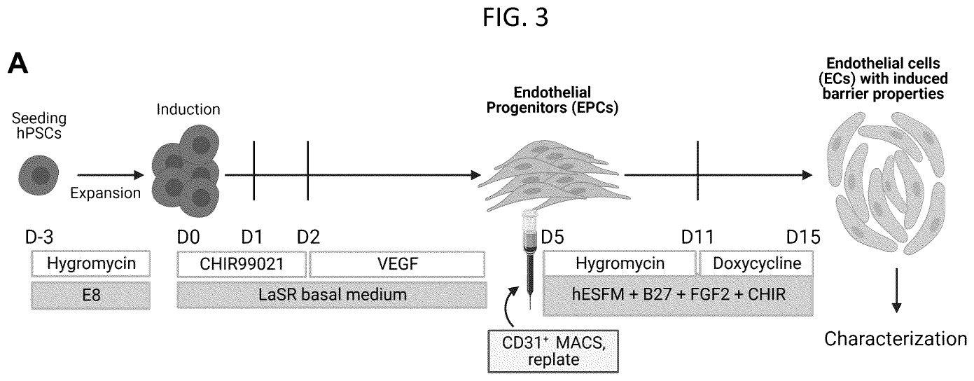

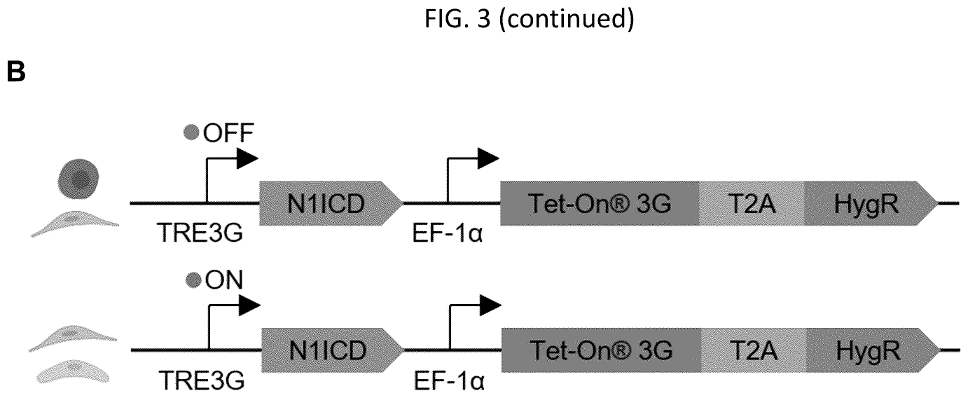

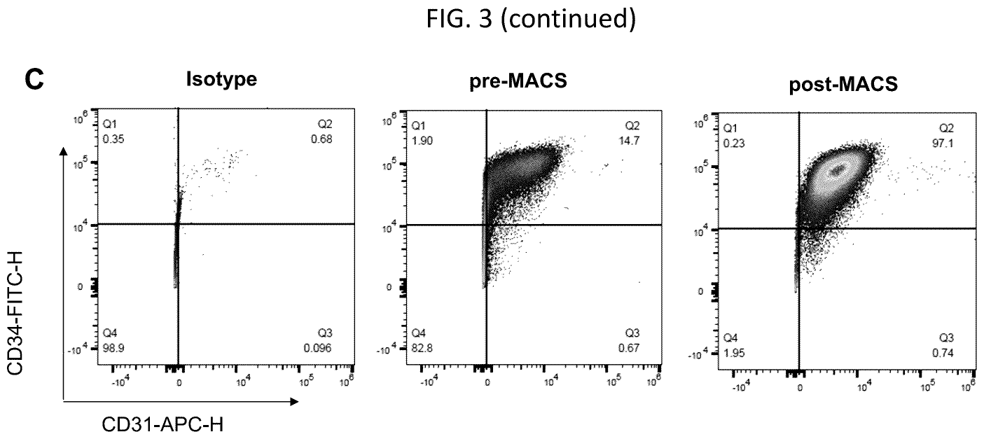

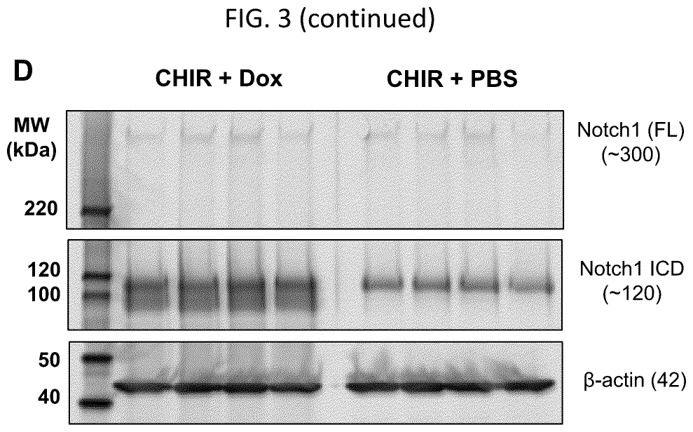

The transgene that the inventors introduced into pluripotent stem cells in the Examples is depicted schematically in FIG. 3B. In addition to the components of the Tet-On system that was used to provide doxycycline inducible gene expression, this transgene also comprises a selectable marker gene that confers resistance to the antibiotic hygromycin. Inclusion of this selectable marker gene allowed the inventors to perform hygromycin selections to select for pluripotent stem cells in which the transgene was successfully inserted in the genome. Thus, in some embodiments, the transgene further comprises a selectable marker gene (i.e., a gene that enables the selection of transformed cells). In some embodiments, the selectable marker gene confers resistance to hygromycin.

### Endothelial Progenitor Cells:

In a second aspect, the present invention provides endothelial progenitor cells that were differentiated from the pluripotent stem cells described herein.

“Endothelial cells” are cells that line blood vessels, lymph vessels, and the heart. They form a semi-permeable barrier between the blood and the surrounding tissues, and regulate the exchange of substances therebetween.

The term “endothelial progenitor cells,” as used herein, refers to CD34+CD31+ cells (i.e., cells that express the cell surface antigens CD34 and CD31) that are able to differentiate into endothelial cells. These cells may also be characterized as CD34+CD31+CD144+ endothelial progenitor cells. Like the pluripotent stem cells from which they were differentiated, the endothelial progenitor cells of the present invention comprise a transgene comprising a polynucleotide encoding a N1ICD operably linked to an inducible promoter.

Endothelial progenitor cells can be isolated from other cell types using any cell sorting method known in the art. Suitable cell sorting methods include both fluorescence activated cell sorting (FACS) methods and magnetic-activated cell sorting (MACS) methods. In the Examples, the inventors isolated endothelial progenitor cells using MACS based on CD31 surface antigen expression. Examples of alternative MACS-based strategies for isolating these cells include using CD34-FITC and anti-FITC magnetic beads with an Easy Sep Magnet and using CD31-biotin and anti-biotin magnetic beads with LS Columns on a MidiMACS Magnet.

Differentiation is the process by which a cell transitions from one cell type into another more specialized cell type. Methods for differentiating pluripotent stem cells into endothelial progenitor cells are described in U.S. Pat. No. 9,290,741, the contents of which are incorporated by reference in their entirety. Briefly, the method for generating endothelial progenitor cells from pluripotent stem cells comprises: (i) contacting cultured pluripotent stem cells with a Wnt/β-catenin signaling activator for a period of about two days in a cell culture medium suitable for maintenance of endothelial cells and substantially free of exogenous growth factors, and then (ii) culturing the cells in the absence of the Wnt/β-catenin signaling activator for about three to ten days. Suitable cell culture medium for maintenance of endothelial cells are known in the art and include, for example, Advanced™ DMEM, VcG-Advanced™ DMEM, LaSR medium, Advanced™ DMEM-F12; VcG-Advanced™ DMEM-F12, StemPro® 34 medium, Advanced™ RPMI, and VcG-Advanced™ RPMI. Other methods for differentiating pluripotent stem cells into endothelial progenitor cells may also be used to generate these cells.

Methods for Producing Endothelial Cells with BBB-Like Transcytosis Properties:

In a third aspect, the present invention provides methods for producing an endothelial cell with BBB-like transcytosis properties. The methods comprise (a) culturing a CD34+CD31+ endothelial progenitor cell in a medium comprising a Wnt/β-catenin signaling activator; and (b) inducing Notch signaling after 2-7 days of culturing.

Cell culture is the process by which cells are grown in an artificial environment. Cells are typically cultured in a culture medium in a vessel (e.g., a dish, flask, plate, or tube), and other factors such as the concentration of gases (e.g., CO₂, O₂), pH, osmotic pressure, and temperature may be manipulated.

A “culture medium” is a substance that provides the necessary nutrients (amino acids, carbohydrates, vitamins, minerals), growth factors, and/or hormones for a cell to grow. A culture medium may be a solid, liquid, or semi-solid substance. In the Examples, the inventors cultured endothelial progenitor cells in hECSR medium to which a Wnt/β-catenin signaling activator was added. “hECSR” is human endothelial serum-free medium (hESFM; i.e., a basal serum-free growth medium) supplemented with B-27 supplement (i.e., a defined yet complex mixture of antioxidant enzymes, proteins, vitamins, and fatty acids) and fibroblast growth factor 2 (FGF2; i.e., a protein that regulates cell growth and differentiation). Thus, in some embodiments, the culture medium comprises hESFM. In some embodiments, the culture medium is supplemented with B-27 supplement and/or FGF2. In specific embodiments, the culture medium is supplemented with 1×B-27 supplement and 20 ng/mL FGF2. Previous findings, described in U.S. Patent Publication No. 2023/0375530, suggest that culturing hPSC-derived endothelial progenitors in commercially available endothelial media (e.g., EGM-2, ECM-5) reduces induction of a BBB-like phenotype and alters the functional properties of the resulting endothelial cells, likely due to the presence of serum proteins and/or mitogens in these media. Thus, while the inventors have not determined whether such commercial media can be used in the methods of the present invention, the composition of the culture media is expected to be important for producing endothelial cells with the desired BBB-like properties, including BBB-like transcytosis properties.

In step (a) of the methods, cells are cultured in the presence of a Wnt/β-catenin signaling activator. As used herein, a “Wnt/β-catenin signaling activator” is any reagent that activates Wnt/β-catenin signaling. Examples of Wnt/β-catenin activators include Wnt ligands and Gsk3 inhibitors. Examples of Wnt ligands include Wnt3a, Wnt7a, and Wnt7b. Wnt ligands may be used at concentrations ranging from about 10 ng/ml to about 200 ng/ml. Examples of Gsk3 inhibitors include small molecules that inhibit Gsk3 phosphotransferase activity or binding interactions, RNA interference reagents that result in Gsk3 knockdown (e.g., SignalSilence® GSK3α/β siRNA), and reagents that result in overexpression of a dominant negative form of Gsk3. Dominant negative forms of Gsk3 are known in the art (see, e.g., Hagen et at. (2002), J Biol Chem, 277 (26): 23330-23335). Suitable small molecule Gsk3 inhibitors include, but are not limited to, CHIR99021 (CAS No. 252917-06-9), CHIR98014 (CAS No. 556813-39-9), BIO-acetoxime (CAS No. 667463-85-6), BIO (CAS No. 667463-62-9), LiCl, SB 216763 (CAS No. 280744-09-4), SB 415286 (CAS No. 264218-23-7), AR A014418 (CAS No. 487021-52-3), 1-Azakenpaullone (CAS No. 676596-65-9), and Bis-7-indolylmaleimide (CAS No. 133052-90-1). In the Examples, the inventors utilized the Gsk3 inhibitor CHIR99021. Thus, in preferred embodiments, the Wnt/β-catenin signaling activator is CHIR99021. CHIR99021 can be used at a concentration ranging from about 3 μM to about 15 μM. In the Examples the inventors utilized CHIR99021 at a concentration of 4 μM. Thus, in preferred embodiments, the concentration of CHIR99021 in the culture ranges from about 3 μM to about 5 μM.

In step (b) of the methods, Notch signaling is induced in the cells after 2-7 days of culturing. The inventors determined that inducing Notch signaling for 2 days was insufficient to produce endothelial cells with BBB-like transcytosis properties, and they found that inducing Notch signaling for 6 days produced endothelial cells with an undesirable endothelial cell morphology and increased risk of cell fate change. Thus, in some embodiments, Notch signaling is induced for 3-6 days. In the Examples, the inventors induced Notch signaling for 4 days. Thus, in preferred embodiments, Notch signaling is induced for 4 days.

Any method for inducing Notch signaling may be used in the present methods. Methods for inducing Notch signaling include, without limitation, expressing a recombinant Notch ligand (e.g., DLL4, JAG2), culturing in a medium comprising a Notch ligand, culturing on extracellular matrix or another substrate endowed with a Notch ligand, co-culturing with cells that overexpress a Notch ligand, artificial activation of DNA binding sites for the transcription factor RBPJ, and overexpression of a full-length Notch receptor or a portion thereof comprising the intracellular domain (e.g., a Notch1 receptor intracellular domain (N1ICD) or Notch3 receptor intracellular domain (N3ICD)). In the Examples, the inventors induced Notch signaling via overexpression of a N1ICD. Thus, in preferred embodiments, Notch signaling is induced via overexpression of a N1ICD. In specific embodiments, Notch signaling is induced via overexpression of a N1ICD comprising or consisting of SEQ ID NO: 3.

As used herein, the term “overexpression” describes expression of a particular protein at levels that are higher than the levels that the protein is natively expressed at. Overexpression can be achieved by introducing one or more additional copies of a polynucleotide encoding a protein into a cell or by editing the endogenous gene encoding that protein such that it is expressed at higher levels.

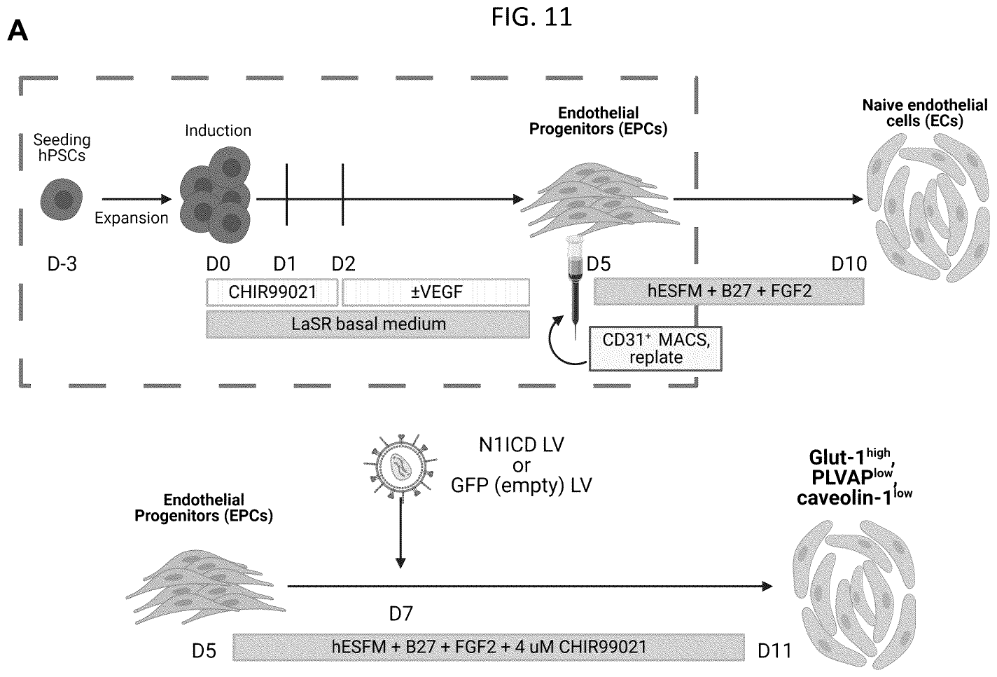

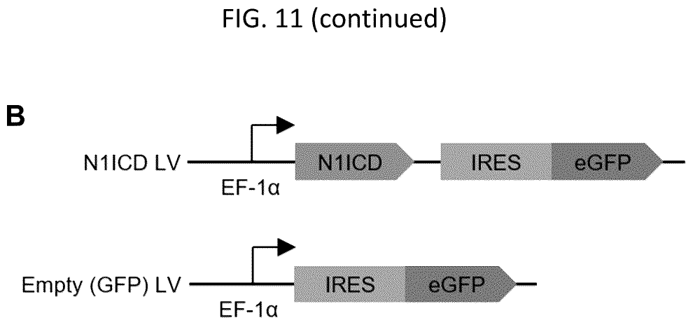

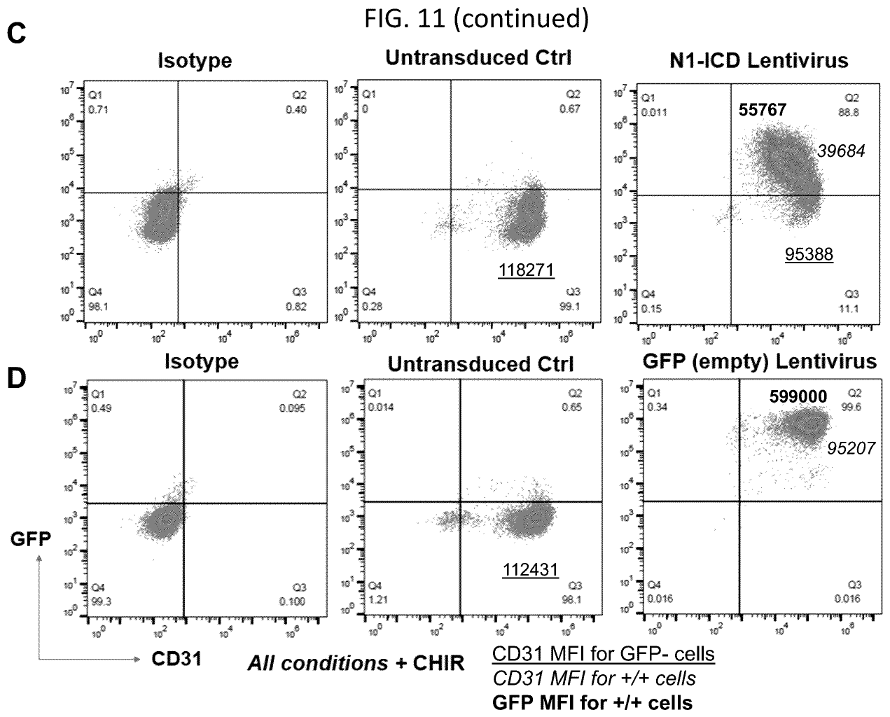

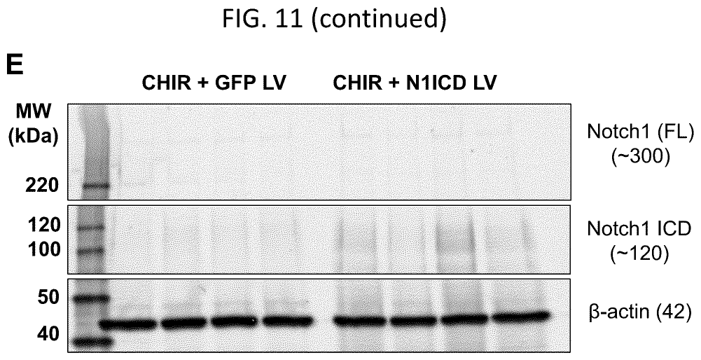

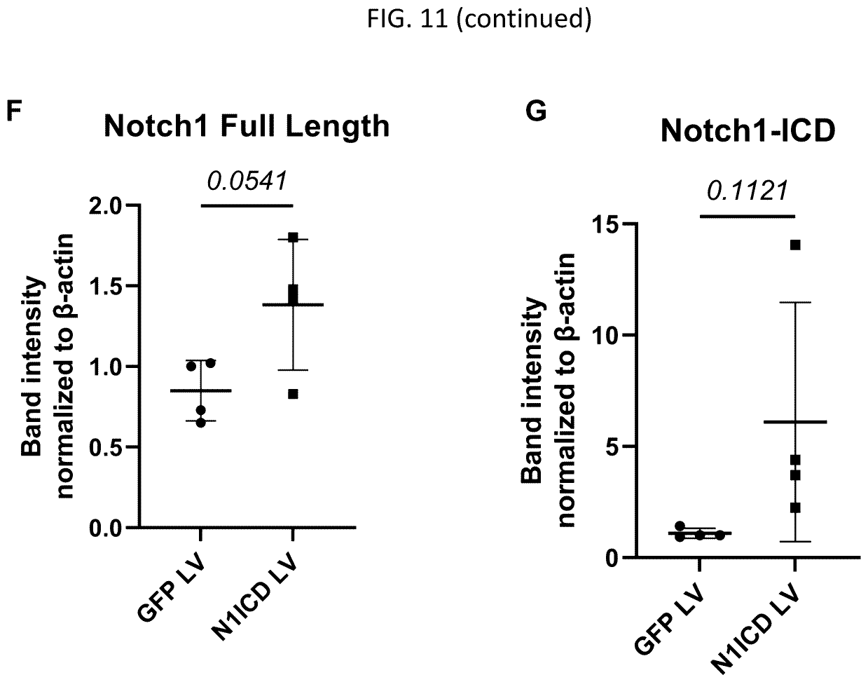

The inventors overexpressed N1ICD in endothelial progenitor cells using two different methods. First, the inventors overexpressed N1ICD from a lentiviral construct. See FIG. 11A for a schematic depiction of this process and see FIG. 11B for a schematic depiction of the N1ICD-encoding construct that was utilized. Thus, in some embodiments, overexpression of N1ICD is achieved via transduction with a virus comprising a polynucleotide encoding N1ICD. “Transduction” is a process in which a virus is used to deliver DNA into a cell. Any virus that is suitable for gene delivery into mammalian cells may be used in the methods of the present invention. Examples of suitable viruses include, without limitation, adenoviruses, adeno-associated viruses, retroviruses, and lentivirus. However, in preferred embodiments, the virus is a lentivirus.

Second, the inventors overexpressed N1ICD by generating gene-edited pluripotent stem cells that comprise a transgene encoding N1ICD under the control of a doxycycline-inducible promoter, differentiating these cells into endothelial progenitor cells, and contacting the differentiated cells with doxycycline to induce N1ICD overexpression. See FIG. 3A for a schematic depiction of this process and see FIG. 3B for a schematic depiction of the N1ICD-encoding construct that was utilized. Thus, in some embodiments, overexpression of the N1ICD is achieved by contacting gene-edited endothelial progenitor cells that comprise a transgene comprising a polynucleotide encoding a N1ICD operably linked to an inducible promoter with a reagent that induces expression from the inducible promoter. In some embodiments, the inducible promoter is a doxycycline-inducible promoter, and the induction reagent is doxycycline.

In embodiments that utilize gene-edited endothelial progenitor cells that comprise a transgene encoding N1ICD, the transgene may further comprise a selectable marker gene, and the methods may further comprise selecting for expression of a selectable marker prior to step (b).

The methods of the present invention produce endothelial cells with BBB-like transcytosis properties. “Transcytosis” is a form of cellular transport in which extracellular cargo is endocytosed, shuttled across the cytoplasm in membrane-bound vesicles, and secreted at a different plasma membrane surface. The term “blood-brain barrier (BBB)-like transcytosis properties” is used herein to describe endothelial cells that express vesicular transcytosis-associated proteins at reduced levels as compared to non-BBB endothelial cells (i.e., endothelial cells that are not part of the BBB). Reduced expression of transcytosis-associated proteins results in reduced levels of transcytosis, like those observed for the brain microvascular endothelial cells (BMECs) that form the BBB in vivo. Examples of transcytosis-associated proteins include caveolin-1, plasmalemma vesicle associated protein (PLVAP), and major facilitator superfamily domain containing 2a (MFSD2A). Caveolae are small lipid rafts that non-specifically shuttle proteins, such as albumin and immunoglobulins, across the brain endothelium. Caveolin-1 is a scaffolding protein that is the main component of caveolae. PLVAP is an endothelial cell-specific protein that forms the diaphragms of caveolae. MFSD2A is a lipid flippase (transporter) expressed in BBB-containing endothelial cells that regulates transport of phospholipids (e.g., docosahexaenoic acid (DHA)). Accumulation of these lipids in the plasma membrane results in suppressed formation of caveolae. In the Examples, the inventors demonstrate that the endothelial cells produced by their methods express lower levels of both caveolin-1 and PLVAP (at both the protein and transcript level). Additionally, they demonstrate that the endothelial cells produced by their methods express higher levels of MFSD2A (at the transcript level). Thus, in some embodiments, the endothelial cell produced by the method exhibits reduced expression of caveolin-1 and/or PLVAP and/or increased expression of MFSD2A relative to a non-BBB endothelial cell. In some embodiments, the endothelial cell produced by the method exhibits reduced expression of caveolin-1 and/or PLVAP and/or increased expression of MFSD2A relative to an endothelial cell produced by performing only step (a) of the method.

Glucose is the primary metabolic fuel used to fuel the mammalian brain, and the glucose transporter GLUT-1 is used to transport glucose across the BBB into the brain. Accordingly, GLUT-1 is expressed at much higher levels in the BMECs that form the BBB than in non-BBB endothelial cells. In the Examples, the inventors demonstrate that the endothelial cells produced by their methods express high levels of GLUT-1 (at both the protein and transcript level). Thus, in some embodiments, the endothelial cell produced by the method exhibits increased expression of GLUT-1 relative to a non-BBB endothelial cell. In some embodiments, the endothelial cell produced by the method exhibits increased expression of GLUT-1 relative to an endothelial cell produced by performing only step (a) of the method.

BMECs also express a battery of brain-specific nutrient transporters with members in the solute carrier (SLC) transporter family that help maintain CNS homeostasis. In the Examples, the inventors demonstrate that expression of many SLC nutrient transport genes is increased in the endothelial cells produced by their methods in response to Notch signaling (at the transcript level). These genes include the neutral amino acid transporters SLC1A4, SLC1A5, and SLC7A5; the cationic amino acid transporters SLC7A1, SLC7A2, and SLC7A6; the multivitamin transporter SLC5A6; and the facilitated glucose transporter SLC2A3. Thus, in some embodiments, the endothelial cell produced by the method exhibits increased expression of SLC1A4, SLC1A5, SLC7A5, SLC7A1, SLC7A2, SLC7A6, SLC5A6, and/or SLC2A3 relative to an endothelial cell produced by performing only step (a) of the method.

Expression levels can be measured at either the protein or transcript level. Examples of methods for measuring expression at the protein level include, but are not limited to, enzyme-linked immunoassay (ELISA), dot blotting, western blotting, flow cytometry, mass spectrometry, 2-D PAGE, and chromatographic methods. Examples of methods for measuring expression at the transcript level include, but are not limited to, reverse transcription polymerase chain reaction (RT-PCR), quantitative PCR, Northern blotting, in situ hybridization, microarray analysis, and RNA sequencing methods.

Additionally, the inventors have demonstrated that the endothelial cells produced by their methods exhibit reduced vesicular endocytosis relative to an endothelial cell produced by performing only step (a) of the method. “Vesicular endocytosis” is a process in which a cell takes in external substances by engulfing them in a vesicle produced via invagination of the cell membrane. Vesicular endocytosis can be measured via an endocytosis assay, such as a fluorescent substrate accumulation assay. In this assay, cellular uptake of fluorescent substrate can be visualized via fluorescence microscopy (e.g., epifluorescence microscopy, confocal microscopy, total internal reflection fluorescence [TIRF] microscopy) or quantified via a flow cytometry-based endocytosis assay. These assays can be performed in live or fixed cells. Vesicular endocytosis can also be visualized via transmission electron microscopy (TEM), in which vesicle structures can be observed at high resolution and counted. Specific types of vesicles, such as caveolin-1 associated vesicles, can be identified in TEM images by immunolabeling vesicle-associated proteins with gold nanoparticle-conjugated antibodies.

In some embodiments, the endothelial progenitor cell used in the method was differentiated from a pluripotent stem cell, as described in the section above titled “Endothelial progenitor cells.”

Endothelial Cells with BBB-Like Transcytosis Properties:

In fourth aspect, the present invention provides populations of endothelial cells with BBB-like transcytosis properties that are produced by the methods described herein. The endothelial cells produced by the methods should also have BBB-like barrier properties, including well-developed tight junctions, selective permeability, moderate transendothelial electrical resistance (TEER), a flat cellular morphology, and junctional architecture characteristic of primary BMECs.

### Bbb Models:

In a fifth aspect, the present invention provides in vitro BBB models comprising a confluent monolayer of the endothelial cells described herein cultured on a surface. The BBB models of the present invention are distinguished from previously described BBB models in that they have BBB-like transcytosis properties.

The BBB models comprise a confluent monolayer of cells. “Confluency” describes the degree to which the surface of a cell culture vessel is covered by adherent cells. Cultured cells are considered “confluent” if the surface is completely covered with cells, and there is no more room for the cells to grow as a monolayer. The term “monolayer” refers to a single layer of cells. Cells in a monolayer grow side-by-side on the same growth surface rather than on top of one another.

Suitable cell growth surfaces are known in the art and include, but are not limited to, permeable supports, collagen coated permeable membranes, filters, polymers, meshes, matrices, membranes, and hydrogel-based substrates. The surface may be within a tissue culture system or a microfluidic device. Examples of suitable permeable supports include tissue culture plate inserts, porous and permeable membranes, and transwell systems (e.g., Corning Transwells®).

In some embodiments, the BBB model is an isogenic model. As used herein, the term “isogenic model” refers to a model made from cells that are selected to model the genetics of a specific subject in vitro. An isogenic BBB model is made by generating induced pluripotent stem cells (iPSCs) from the subject's somatic cells (i.e., by reprogramming them to a pluripotent state), differentiating the iPSCs into endothelial progenitor cells, using the methods described herein to differentiate the endothelial progenitor cells into endothelial cells with BBB-like transcytosis properties, and culturing the resulting cells on a suitable surface to generate a BBB model.

The “subject” may be a mammal or a non-mammalian vertebrate, such as a bird. Suitable mammals include, but are not limited to, humans, cows, horses, sheep, pigs, goats, rabbits, dogs, cats, bats, mice, and rats. In certain embodiments, the subject is a lab animal (e.g., a mouse or rat) and the BBB model is used for research purposes. In preferred embodiments, the subject is a human.

In some embodiments, the BBB model is an isogenic model generated from the cells of a subject that has a brain disease. In these embodiments, the BBB model can be used to screen for therapeutics that are able to cross the subject's BBB to treat the brain disease. Examples of brain diseases include, without limitation, Alzheimer's disease, multiple sclerosis, stroke, epilepsy, traumatic brain injury, Parkinson's disease, and brain tumors.

### Methods for Using the BBB Models:

In a sixth aspect, the present invention provides methods for using the BBB models described herein. The BBB models can be used as a research tool or for pre-clinical studies of trans-BBB transport of therapeutic agents.

A “therapeutic agent” is an agent that aids in the treatment, prevention, or diagnosis of a disease or condition. Examples of therapeutic agents include pharmaceuticals, biologics, toxins, alkylating agents, enzymes, antibiotics, antimetabolites, antiproliferative agents, chemotherapeutic agents, hormones, neurotransmitters, oligonucleotides, aptamers, lectins, compounds that alter cell membrane permeability, photochemical compounds, small molecules, liposomes, micelles, gene therapy vectors, and vaccines.

In some embodiments, the therapeutic agent is an agent that needs to cross the BBB to reach its target for therapy or to have a therapeutic effect. In some cases, the therapeutic agent may use or be suspected of using the caveolae-mediated transport pathway to cross the BBB. Examples of such agents include albumin-bound nanoparticle drugs (e.g., nab-paclitaxel (Abraxane)) and liposomal nanocarriers. Although caveolar-mediated transcytosis is normally suppressed at the BBB as in the BBB model system provided herein, it is upregulated in the early stages of several CNS diseases (e.g., glioblastoma multiforme) and is a hallmark of aging (Sorets et al., Curr Opin Chem Eng 30: 86-95, 2020). Thus, this pathway could potentially be utilized to deliver therapeutic agents to the CNS under such conditions or following transient BBB disruption via focused ultrasound (FUS).

The ability of a therapeutic agent to cross the BBB can be assessed using a transwell assay. In this assay, the BBB model comprises a confluent monolayer of endothelial cells cultured on a porous membrane that separates two chambers, and the transport of substances between the two chambers is measured. The BBB models provided herein, which have BBB-like transcytosis properties, will more accurately reflect the ability of therapeutic agents to cross the BBB in vivo.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

Induction of blood-brain barrier (BBB) properties in central nervous system (CNS) endothelial cells during human development is incompletely understood and our knowledge of this process is derived mainly from animal models^{1,2}. In vitro BBB models derived from human pluripotent stem cells (hPSCs) can be used to study human BBB development, cerebrovascular disease, and therapeutic approaches for treating brain diseases³. However, there is a limited understanding of signaling pathways that influence the unique property of low vesicular endocytosis and transcytosis in brain microvascular endothelial cells relative to peripheral endothelial cells. However, in vivo studies^{4,5} suggest the importance of certain BBB-relevant signaling pathways, including Wnt and Notch, to the induction of this feature of the BBB.

In the following example, the inventors differentiated endothelial progenitor cells (EPCs) from hPSCs, treated the resulting hPSC-EPCs with CHIR99021 to activate the Wnt/β-catenin signaling cascade, and overexpressed the Notch1 receptor intracellular domain (N1ICD) to simulate Notch signaling. They show that co-activation of Notch and Wnt/β-catenin signaling resulted in simultaneous upregulation of GLUT-1, a BBB-enriched glucose transporter, and decreased expression of both PLVAP and caveolin-1⁶, two vesicular transcytosis-associated proteins. They also discovered that the combination of CHIR99021 and N1ICD overexpression can induce significant upregulation of MFSD2A, which encodes a lipid flippase associated with reduced vesicular transcytosis across the BBB⁷. While Wnt/β-catenin signaling has previously been shown to drive hPSC-EPCs to a more CNS-like phenotype⁸, the substantial additional effects of Notch signaling on transcytosis properties were unknown. These findings suggest that this combination of signaling inputs results in induction of important barrier properties, including expression of BBB-enriched nutrient transporters, reduced expression of transcytosis proteins, and reduced vesicle-based substrate uptake. These findings contribute to our understanding of developmentally relevant signaling pathways and their importance to induction of specific BBB properties.

### INTRODUCTION

Brain microvascular endothelial cells (BMECs) are specialized vascular endothelial cells that make up all central nervous system (CNS) microvasculature with the exception of vessels of the circumventricular organs^{9,10}. BMECs are characterized by blood-brain barrier (BBB) properties that distinguish them from similarly sized vessels in other organs due to their restriction of passage of most solutes and cells from the vessel lumen into the central nervous system^{1,2}. These properties include expression of tight junction proteins including claudin-5 and occludin, which seal cell-cell junctions and restrict paracellular transport^{11,12}. BMECs also express a battery of brain-specific nutrient transporters with members in the solute carrier (SLC) transporter family that help maintain CNS homeostasis¹³. Polarized efflux pumps with broad-spectrum recognition of small molecules in the ATP-binding cassette (ABC) family prevent the entry of majority of pharmaceuticals¹³, except certain anesthetic, psychiatric drugs, and anti-epileptic drugs^{14,15}. Finally, a major property of BMECs is the extremely low levels of transcytosis compared to vascular endothelial cells in peripheral organs^{6,7}.

However, much of our understanding of how these properties arise in the human CNS remains limited; most of our knowledge about signaling pathways and important regulators of BBB property induction and maintenance has been deduced through genetic studies in model animals, particularly mice². These signaling pathways include canonical Wnt, VEGF, Notch, TGF-β, and Sonic hedgehog (Shh) signaling as well as other pathways^{1,16,17}. The role of Wnt signaling in CNS angiogenesis and barrier-genesis (or the establishment of barrier properties in CNS endothelium) has been most prolifically studied^{8,18-20}.

Notch signaling is a pathway of intrinsic importance to endothelial cell function. It is involved in the regulation of arterial differentiation of endothelial cells and sprouting angiogenesis²¹. Laminar flow-induced signaling through the NOTCH1 receptor in arterial vessels is important for maintenance of endothelial cell-cell junctions and reduction of endothelial proliferation²². Notch signaling also mediates interactions between mural and endothelial cells at the blood-brain barrier²³. An undetermined ligand on the surface of CNS pericytes binds to the NOTCH1 receptor on the surface of BMECs to liberate the NOTCH1 intracellular domain (N1ICD). The N1ICD interacts with SMAD4 which results in upregulation of N-cadherin and maintenance of pericyte-BMEC adhesions²⁴. When we examined endothelial cells using an integrative single cell RNA-seq comparison of human brain and peripheral vasculature²⁵, we found that NOTCH1 and genes encoding the Notch signaling-related transcription factors HES1/4 were enriched in brain endothelial cells compared to peripheral endothelial cells.

A recent study⁴ of the retinal vasculature in adult mouse models demonstrated the importance of Notch signaling, specifically the interaction between Notch ligand D114 and the receptor Notch1 in the maintenance of low levels of vesicular transcytosis that characterize the blood-retina barrier (BRB). Antibody-mediated blockade of the Notch ligand-receptor interaction in this context resulted in increased expression of vesicular transcytosis-associated structural proteins caveolin-1 and PLVAP, increased numbers of endothelial vesicles, and increased permeability of retinal vasculature to both 4 and 10 kDa fluorescent Dextran⁴. It is unknown based on existing evidence whether Notch signaling regulates MFSD2A, a lipid flippase enriched in BMECs that is tied to reduced levels of caveolae-mediated transport at the BBB^{5,7,26,27}.

Notch signaling is mediated by signals from a “sending” cell via one of five transmembrane Notch ligands (e.g., Delta-like (DLL) 1, 3, and 4 or Jagged (JAG) 1 and 2) to a “receiving” cell expressing one of 4 Notch receptors (NOTCH 1-4)²¹. Multiple Notch ligands are capable of binding to and activating signaling through an individual Notch receptor variant²⁸. Following the ligand-receptor interaction at the cell surface, sequential enzymatic cleavage events by ADAM (a disintegrin and metalloproteinase domain-containing protein) and γ-secretase ultimately result in liberation of the Notch intracellular domain (NICD), the main transcriptional effector of the canonical Notch signaling pathway^{21,29,30}. NICD then translocates to the nucleus where it complexes with transcription factor RBPJ (also known as CBF-1 or CSL) and co-activator MAM to regulate expression of downstream genes²⁹. Canonical Notch signaling is dependent on the DNA binding protein RBPJ, whereas non-canonical Notch signaling is RBPJ-independent³¹. Non-canonical Notch signaling includes interactions of NICD in the cytoplasm with effectors of the Wnt, PI3K, mTORC2, or AKT signaling pathways or in the nucleus with transcription factors such as HIF-1α, NFκB, or YY1³¹.

Because of the molecular and functional similarities between the BRB and the BBB, we hypothesized that overactivation of the Notch signaling pathway in an in vitro hPSC-derived endothelial model system is important for induction of certain BBB properties, including reduction of vesicular transport. To our knowledge, there are no small-molecule agonists that directly target Notch signaling without off-target effects. While N-methylhemeanthidine chloride (NMHC)³², chrysin³³ and hesperetin³⁴ have been identified as potential Notch signaling agonists, they are cytotoxic and can interact with other pathways, including AKT signaling by NMHC³⁵ and TGF-β and NFκB signaling by hesperitin^{36,37}. Another reported activator of Notch signaling is adropin³⁸, a protein that interacts with the little-studied non-canonical NOTCH1 ligand NB-3 to promote oligodendrocyte progenitor differentiation through a Deltex1 (DTX1)-mediated pathway³⁹. DTX1 expression is essentially absent in hPSC-derived endothelial progenitor cells (hPSC-EPCs)⁸, so adropin-mediated Notch signaling activation is unlikely to be relevant in our experimental system. Therefore, we chose to directly activate Notch signaling by overexpressing N1ICD, a gene encoding the intracellular transcriptional effector of signaling through the NOTCH1 receptor. This approach bypasses any off-target effects of other agonist molecules that are not directly related to Notch signaling. We show that, in Wnt signaling-activated hPSC-EPCs (supplemented with a low dose of CHIR99021), Notch signaling activation results in BBB-like expression changes in MFSD2A, caveolin-1, PLVAP, and GLUT-1.

### Materials and Methods:

hPSC Maintenance

Matrigel-coated tissue culture plates were prepared by thawing and resuspending a 2.5 mg aliquot of Matrigel, Growth Factor Reduced (Corning, Glendale, AZ) in 30 mL DMEM/F-12 (Life Technologies, Carlsbad, CA). The Matrigel solution was used to coat tissue culture plates (Corning) at a concentration of 8.7 μg/cm² (1 mL/well in a 6-well plate, 0.5 mL/well in a 12-well plate). A 2.5 mg Matrigel aliquot dissolved in DMEM/F-12 is sufficient to coat up to five 6- or 12-well plates. Matrigel-coated plates were then incubated at 37° C. for a minimum of 1 h, maximum of 1 week before use. hPSCs used in this study were IMR90-4 iPSCs (WiCell, Madison, WI). hPSCs were maintained on Matrigel-coated 6-well plates in E8 medium (STEMCELL Technologies, Vancouver, Canada) at 37° C., 5% CO₂ with daily media changes.

hPSCs were passaged with Versene (Life Technologies) at 70-80% confluence or when colonies began to touch. To passage, hPSCs were washed once with Versene, then incubated with Versene for 7-8 min. Next, Versene was aspirated and hPSC colonies were dissociated by gentle spraying with 4 mL E8 medium. To achieve a 1:12 split ratio, 2 mL of the hPSC-containing E8 suspension was transferred to a conical tube containing 4.2 mL E8. For a 1:6 split ratio, all 4 mL of the hPSC-containing E8 suspension was transferred to conical tube containing 2.2 mL E8. Next, for both split ratios, the cell suspension was gently mixed by lifting and lowering a serological pipette 5-10 times (without pipetting up and down). In a 6-well Matrigel-coated plate pre-filled with 1 mL/well E8, 1 mL/well of the mixed hPSC suspension was equally distributed, 0.5 mL per step in a U-shaped fashion, to achieve a final volume of 2 mL/well. The plate was subsequently agitated from left to right and front to back 3-4 times each and incubated at 37° C., 5% CO₂ for 24 h without disturbing before the next media change.

Differentiation of hPSC-Derived Endothelial Progenitor Cells (hPSC-EPCs)

Both gene-edited and unedited hPSCs were differentiated to EPCs according to previously published protocols^{8,40,41} with minor modifications. On day −3 (D-3), hPSCs were treated with 1 mL/well Accutase (Innovative Cell Technologies, San Diego, CA) (1 mL/well) for 7 min at 37° C. Accutased hPSCs were triturated to completely singularize cells and subsequently quenched in 4× volume of E8 (STEMCELL Technologies). Singularized hPSCs were counted with a hemacytometer and subsequently centrifuged for 5 min, 200×g. The hPSC pellet was resuspended in E8 supplemented with 10 μM ROCK inhibitor Y-27632 (Tocris, Bristol, UK) and seeded onto Matrigel-coated 12-well plates at a density of (3-5)×10⁴ cells/cm², 1 mL/well. Plates were incubated at 37° C., 5% CO₂. The following 2 days (D-1 and D-2), media was changed to E8, 1 mL/well. On D0, differentiation media was added with LaSR medium (Advanced DMEM/F-12 [Life Technologies], 2.5 mM GlutaMAX [Life Technologies], and 60 μg/mL L-ascorbic acid 2-phosphate magnesium [Sigma-Aldrich, St. Louis, MO]) supplemented with 6 μM CHIR99021 (henceforth abbreviated as “CHIR”, Tocris), 2 mL/well. On D1, media was changed with LaSR supplemented with 6 μM CHIR, 2 mL/well. Media changes on D1 and D2 were performed 24 h±30 min after the previous media change. From D2-D4, media was changed with pre-warmed 37° C. LaSR supplemented with 50 ng/mL recombinant human VEGF₁₆₅ (Peprotech, Cranbury, NJ), 2 mL/well.

On D5, hPSC-EPCs were sorted by magnetic-activated cell sorting (MACS) based on CD31 surface antigen expression. hPSCs were dissociated with 1 mL/well Accutase for 15 min, 37° C. 12 mL/plate of Accutased cells were thoroughly singularized by trituration and quenched through a 40 μm cell strainer (Beckton Dickinson, Vernon Hills, IL) in 38 mL DMEM (Life Technologies) supplemented with 10% FBS (R&D Systems, Minneapolis, MN). Quenched hPSCs were counted with a hemocytometer and centrifuged for 5 min, 200×g. Pelleted cells were resuspended in MACS buffer (Dulbecco's phosphate buffer saline without Ca and Mg [DPBS; Life Technologies], 0.5% w/v bovine serum albumin [Sigma-Aldrich], 2 mM EDTA [Sigma-Aldrich]) at a concentration of 60 μL/10⁷ cells. Human FcR blocking reagent (Miltenyi Biotec, Auburn, CA) was added at a dilution of 1:50, and CD31 magnetic microbeads (Miltenyi Biotec) were added at a dilution of 20 μL/10⁷ cells, gently mixed by pipetting, incubated for 15 min, 4° C. Cells were then washed by adding 2 mL MACS buffer/10⁷ cells and centrifuging for 5 min, 200×g. During centrifugation, a MidiMACS magnetic separator (Miltenyi Biotec) was prepared in the BSC by placing LS columns (Miltenyi Biotec) into available slots with “fins” facing away from the magnet. The LS columns were primed with 3 mL MACS buffer. Next, the cell pellet was resuspended in 0.5-2 mL MACS buffer depending on number of LS columns (1-4, respectively). 0.5 mL of cell suspension was loaded per LS column (about (4-6)×10⁷ cells per column). After cell suspension had completely flowed through the LS column, 3 additional MACS buffer washes (3 mL/column) were performed, allowing wash buffer to stop dripping completely before adding next wash fraction. After final wash, each column was removed the MidiMACS magnet, and cells were eluted with 5 mL MACS buffer and the provided plunger into a sterile conical tube. Eluate fractions were combined; cells were counted by hemacytometer and centrifuged for 5 min, 200×g. A portion of the hPSC-EPCs were used for CD31/CD34 flow cytometry validation of pre-MACS and post-MACS purity. The remaining cells were either directly used for experiments or cryopreserved in aliquots of 2.5×10⁶ cells/mL in EPC freezing medium. EPC freezing medium is 60% hECSR (human endothelial serum-free medium [hESFM; Life Technologies] supplemented with 1×B-27 supplement [Life Technologies] and 20 ng/ml FGF2 [Waisman Biomanufacturing, Madison, WI]), 30% FBS, 10% dimethyl sulfoxide (DMSO; Sigma-Aldrich) passed through a 0.22 μm pore Steriflip (Sigma-Aldrich) filter.

### Production of Lentivirus

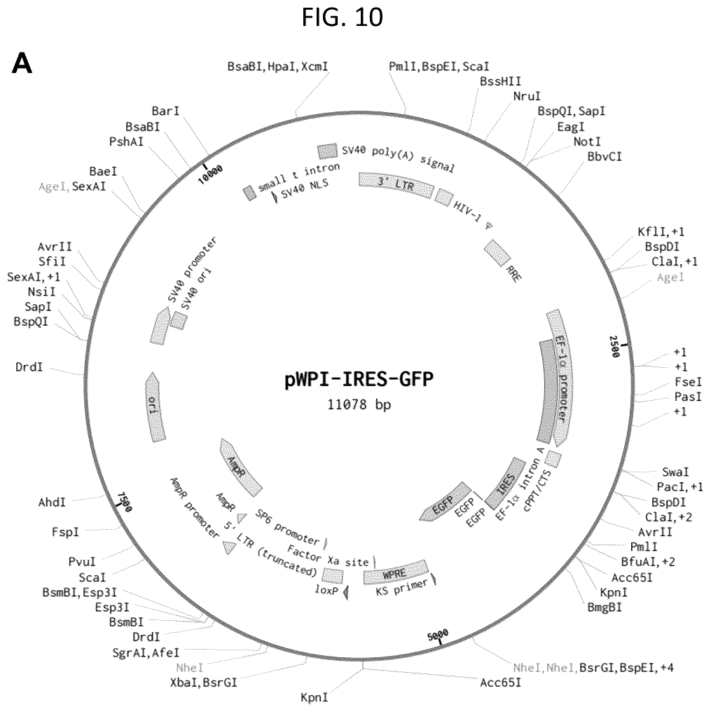

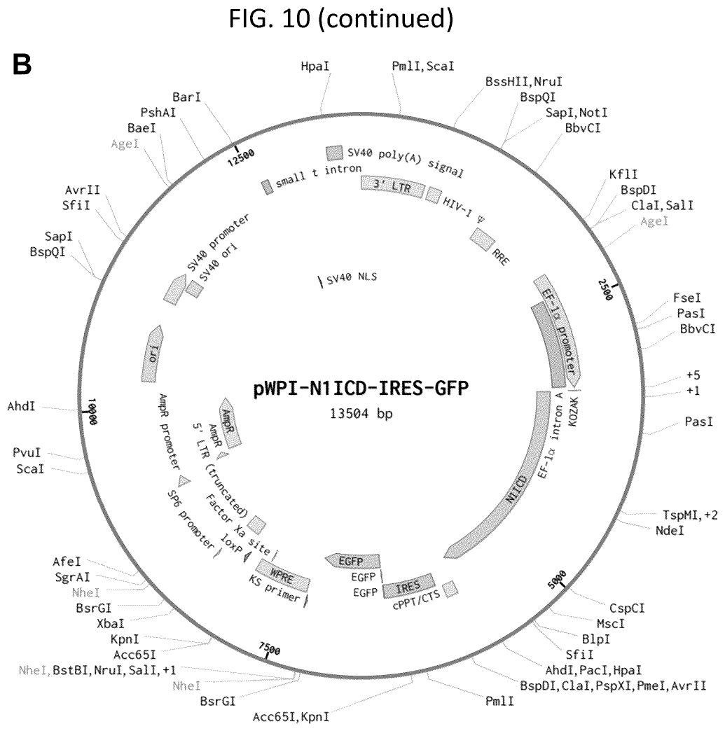

Lentivirus plasmids pWPI (FIG. 10A, Addgene plasmid #12254), psPAX2 (Addgene plasmid #12260), and pMD2.G (Addgene plasmid #12259) were obtained from Addgene (Watertown, MA) as gifts from Didier Trono. To generate transfer plasmid pWPI-N1ICD (FIG. 10B), a construct encoding the native human Notch1 intracellular domain (N1ICD) was cloned into the pWPI plasmid after the EF-1α promoter and ahead of the IRES cassette. First, a cDNA fragment encoding the intracellular domain of the Notch1 receptor was amplified from a cDNA library generated from an hPSC-derived neural crest cells⁴². This fragment spans nucleotides 5,522-7,930 of NM_017617.5, corresponding to amino acids 1,754-2,556 of NP060087.3. For N1ICD, the forward primer (Table 1) contained a Kozak consensus sequence and start codon; forward and reverse primers (Table 1) included PacI restriction enzyme sites. pWPI and the resulting PCR fragments were digested with PacI. Ligation was performed with Instant Sticky-end Ligase Master Mix (New England Biolabs, Ipswich, MA), and the resulting products were transformed into NEB Stable Competent E. coli (New England Biolabs). Single ampicillin-resistant colonies were picked and screened via PCR for presence of insert using primers annealing to the EF-1α promoter and IRES (Table 1). Clones with forward-oriented inserts were identified and the correct sequence was confirmed via Sanger Sequencing. pWPI and pWPI-N1ICD plasmids were then expanded and purified using the EndoFree Plasmid Maxi Kit (Qiagen, Germantown, MD). pWPI-N1ICD has been deposited to Addgene (Addgene plasmid #185525).

TABLE 1
Primers

| | |
|---|---|
| NAME | SEQUENCE |

Primers for cloning

| | |
|---|---|
| NOTCH1 forward | TAA GCA TTA ATT AAG CCA CCA TGG TGC TGC TGT CCC |
| | GCA AGC G (SEQ ID NO: 7) |
| NOTCH1 reverse | TGC TTA TTA ATT AAT TAC TTG AAG GCC TCC GGA A |
| | (SEQ ID NO: 8) |
| EF-1α promoter | TCA AGC CTC AGA CAG TGG TTC (SEQ ID NO: 9) |
| forward | |
| IRES reverse | CCT CAC ATT GCC AAA AGA CG (SEQ ID NO: 10) |
| NheI overhang for | ACT AAA GCT AGC TGT CGT GAG GAA TTT CGA CAT TT |
| N1ICD (SB001) | (SEQ ID NO: 11) |
| AgeI overhang for | ACT AAA ACC GGT CCC CCT TTT CTT TTA AAA GTT AAC |
| N1ICD (SB002) | CG (SEQ ID NO: 12) |

Primers for Sanger sequencing

| | |
|---|---|
| TRE3G promoter | GTA CGG TGG GCG CCT ATA AA (SEQ ID NO: 13) |
| forward (BD69) | |
| N1ICD reverse | GGG CAC CGT CTG AAG CGT TCT T (SEQ ID NO: 14) |
| (SB003) | |
| N1ICD forward | GCA GTG GAC TCA GCA GCA CCT G (SEQ ID NO: 15) |
| (SB004) | |
| N1ICD reverse | GCG GCC CAT GTT GTC CTG GAT G (SEQ ID NO: 16) |
| (SB005) | |
| N1ICD forward | GAG GGC ATG CTG GAG GAC CTC A (SEQ ID NO: 17) |
| (SB006) | |
| N1ICD forward | GGA AGC AAG GAG GCC AAG GAC C (SEQ ID NO: 18) |
| (SB007) | |
| N1ICD reverse | GTG AAA TTC AGG GCC CCT CCG C (SEQ ID NO: 19) |
| (SB008) | |
| N1ICD forward | CCC TGC AGC ATG GCA TGG TAG G (SEQ ID NO: 20) |
| (SB009) | |
| N1ICD reverse | GGG GCT CTC CTG GGG CAG AAT A (SEQ ID NO: 21) |
| (SB010) | |
| PB-TRE backbone | GGG TAT CGA CAG AGT GCC AG (SEQ ID NO: 22) |
| reverse (BD70) | |

Primers for RT-qPCR

| | |
|---|---|
| ABCB1 Taqman probe | Hs00184500_m1 |
| MFSD2A Taqman | Hs00293017_m1 |
| probe | |
| ABCC1 forward | CTG AGT TCC TGC GTA CCT ATG (SEQ ID NO: 23) |
| ABCC1 reverse | TGC CAT TCT CCA TTT GCT TTG (SEQ ID NO: 24) |
| ABCG2 forward | CTC AGA TCA TTG TCA CAG TCG T (SEQ ID NO: 25) |
| ABCG2 reverse | GTC GTC AGG AAG AAG AGA ACC (SEQ ID NO: 26) |
| ACTB forward | ACA GAG CCT CGC CTT TG (SEQ ID NO: 27) |
| ACTB reverse | CCT TGC ACA TGC CGG AG (SEQ ID NO: 28) |
| CAV1 forward | CAT GGC AGA CGA GCT GAG (SEQ ID NO: 29) |
| CAV1 reverse | AAA CTG TGT GTC CCT TCT GG (SEQ ID NO: 30) |
| CDH2 forward | CTC CAA TCA ACT TGC CAG AA (SEQ ID NO: 31) |
| CDH2 reverse | ATA CCA GTT GGA GGC TGG TC (SEQ ID NO: 32) |
| CDH5 forward | GAA CCA GAT GCA CAT TGA TGA AG (SEQ ID NO: 33) |
| CDH5 reverse | TGC CCA CAT ATT CTC CTT TGA G (SEQ ID NO: 34) |
| CLDN5 forward | TGA CCT TCT CCT GCC ACT A (SEQ ID NO: 35) |
| CLDN5 reverse | AAG CGA AAT CCT CAG TCT GAC (SEQ ID NO: 36) |
| HEYL forward | CAC TTG AAA ATG CTC CAT GCC (SEQ ID NO: 37) |
| HEYL reverse | ACT CCC GAA AAC CAA TGC TC (SEQ ID NO: 38) |
| HPRT1 forward | TTG TTG TAG GAT ATG CCC TTG A (SEQ ID NO: 39) |
| HPRT1 reverse | GCG ATG TCA ATA GGA CTC CAG (SEQ ID NO: 40) |
| FN1 forward | TGG TGT CAC AGA GGC TAC TA (SEQ ID NO: 41) |
| FN1 reverse | GGG CTC GCT CTT CTG ATT ATT (SEQ ID NO: 42) |
| GAPDH forward | ACA TCG CTC AGA CAC CAT G (SEQ ID NO: 43) |
| GAPDH reverse | TGT AGT TGA GGT CAA TGA AGG G (SEQ ID NO: 44) |
| GFP forward (SB012) | TCG CCG ACC ACT ACC AGC AGA A (SEQ ID NO: 45) |
| GFP reverse (SB013) | CGC GCT TCT CGT TGG GGT CTT T (SEQ ID NO: 46) |
| N1ICD forward | GCA GTG GAC TCA GCA GCA CCT G (SEQ ID NO: 47) |
| (SB004) | |
| N1ICD reverse | CTG CAG GAG GCG ATC ATG AGC G (SEQ ID NO: 48) |
| (SB011) | |
| OCLN forward | ATG GCA AAG TGA ATG ACA AGC (SEQ ID NO: 49) |
| OCLN reverse | AGG CGA AGT TAA TGG AAG CTC (SEQ ID NO: 50) |
| PLVAP forward | TGG ACA CCT GCA TCA AGA C (SEQ ID NO: 51) |
| PLVAP reverse | GGA TCT TCC TCT TGA ACT CCT C (SEQ ID NO: 52) |
| SLC2A1 forward | GTG CCA TAC TCA TGA CCA TCG (SEQ ID NO: 53) |
| SLC2A1 reverse | GGC CAC AAA GCC AAA GAT G (SEQ ID NO: 54) |
| TJP1 forward | CGC GTC TCT CCA CAT ACA TTC (SEQ ID NO: 55) |
| TJP1 reverse | GCT GGC TTA TTC TGA GAT GGA (SEQ ID NO: 56) |

Lentivirus comprising the constructs N1ICD-IRES-GFP (denoted N1ICD LV) and IRES-GFP negative control (denoted GFP LV) were produced in HEK293TN cells (System Biosciences, Palo Alto, CA). 293TN cells were maintained on uncoated 6-well tissue culture plates in DMEM (Life Technologies) supplemented with 10% FBS (R&D Systems, Minneapolis, MN), 1 mM sodium pyruvate (Life Technologies), and 0.5× GlutaMAX Supplement (Life Technologies), with media changes every other day. When 293TN cells reached 70-90% confluence, they were transfected with the packaging plasmids psPAX2 (1 μg/well) and pMD2.G (0.5 μg/well), and the transfer plasmid pWPI-N1ICD or pWPI (1.5 μg/well) using FuGENE HD Transfection Reagent (Promega, Madison, WI). Media was supplemented with 1× antibiotic-antimycotic (Life Technologies) on the day of transfection. Approximately 16-18 h later, media was aspirated and replaced with fresh pre-warmed HEK293TN media, and virus-containing supernatants were collected 24, 48, and 72 h later. After the 1^{st} and 2^{nd} collections, media was replaced with fresh pre-warmed 293TN medium and virus-containing supernatant was cooled at 4° C. After the final collection, the 3^{rd} virus-containing supernatant fraction was cooled at 4° C. for a minimum of 30 min to overnight. The three virus-containing supernatants were then combined, centrifuged to remove cell debris, and passed through a 0.45 μm pore Steriflip filter (Millipore SE1M003M00) before being concentrated 100× with Lenti-X Concentrator (Takara Bio, Mountain View, CA). Briefly, Lenti-X/media mixture was incubated at 4° C. for 30 mins before centrifuging at 1,500×g & 4° C. for 45 mins. Subsequently, supernatant was aspirated, and lentivirus-containing pellets were resuspended in 200 μL each of sterile DPBS and subsequently combined to form a single consistent stock. 25-50 μL aliquots were prepared and frozen at −80° C. for long-term storage.

Culture of hPSC-EPCs:

Collagen IV (Sigma-Aldrich) was dissolved in 0.5 mg/mL acetic acid to a final concentration of 1 mg/mL. Collagen IV-coated plates were made by diluting a volume of the 1 mg/mL stock solution 1:100 in sterile water and adding the resulting solution to tissue culture plates. 1 mL collagen IV solution/well was added to 6-well plates (9.5 cm²/well) and the volume of solution was scaled according to surface area in other multi-well plate formats (e.g., 100 μL/well for a 48-well plate [0.95 cm²/well]). After adding collagen IV solution, the tissue culture plates were incubated for 1 h at RT, protected from light. Collagen IV coating solution was then removed, and cryopreserved hPSC-EPCs were thawed and resuspended in hECSR medium and plated at approximately 4×10⁴ cells/cm². hPSC-EPCs were incubated at 37° C., 5% CO₂ with hECSR medium changes every other day. In some experiments, small molecules and/or lentivirus were added to hECSR medium: in all cases, 4 μM CHIR (Tocris) was added to the culture medium to activate Wnt/β-catenin signaling. To activate Notch signaling, we overexpressed N1ICD using lentiviral transduction in unedited hPSC-derived EPCs or added doxycycline to gene-edited EPCs comprising a tetracycline-inducible N1ICD cassette.

### Culture of HUVECs

Human umbilical vein endothelial cells (HUVECs), pooled in EGM-2 (Lonza, Basel, Switzerland), were thawed and plated at a seeding density of 2500 cells/cm² in an uncoated T-75 flask. Cells were incubated at 37° C., 5% CO₂ and media was replaced every other day with EGM-2 medium (EBM-2 medium [Lonza] supplemented with EGM-2 Endothelial SingleQuots Kit [Lonza]). When cells reached 80-90% confluency, cells were incubated with ˜5 mL 0.25% Trypsin-EDTA with phenol red (Life Technologies) for 10 min at 37° C. and dissociated by trituration. Trypsinized HUVECs were quenched in 4× the volume of EGM-2 medium and centrifuged at 200×g for 5 mins. The cell pellet was resuspended in EGM-2 to a concentration that would achieve a seeding density of 2500 cells/cm² in an uncoated 6-well plate. Cells were cultured for 2 days before replacing EGM-2 medium supplemented with 1.9 μL/well GFP LV or 50 L/well N1ICD LV. Media was changed every other day with EGM-2 medium. 6 days after lentiviral transduction, cells were dissociated and stained for CD31 or caveolin-1 via flow cytometry or lysed for qPCR analysis.

To study substrate accumulation in HUVECs, cells were plated at a seeding density of 5000 cells/cm² in each well of 2 12-well plates with 1 additional well in a third 12-well plate. Cells were incubated at 37° C., 5% CO₂ and media was replaced every other day with EGM-2 medium. Cells were cultured for 2 days before the media was replaced with EGM-2 medium supplemented with 0.76 μL/well GFP LV or 20 μL/well N1ICD LV (doses of both types of lentivirus scaled down by a factor of 2.5× from 6-well plate). Media was changed every other day with EGM-2 medium. 6 days after lentiviral transduction, cells were pretreated with or without endocytosis inhibitors, incubated with or without fluorescent albumin, imaged by epifluorescence microscopy, and isolated for flow cytometry analysis.

Lentiviral Overexpression of N1ICD in Unedited hPSC-EPCs:

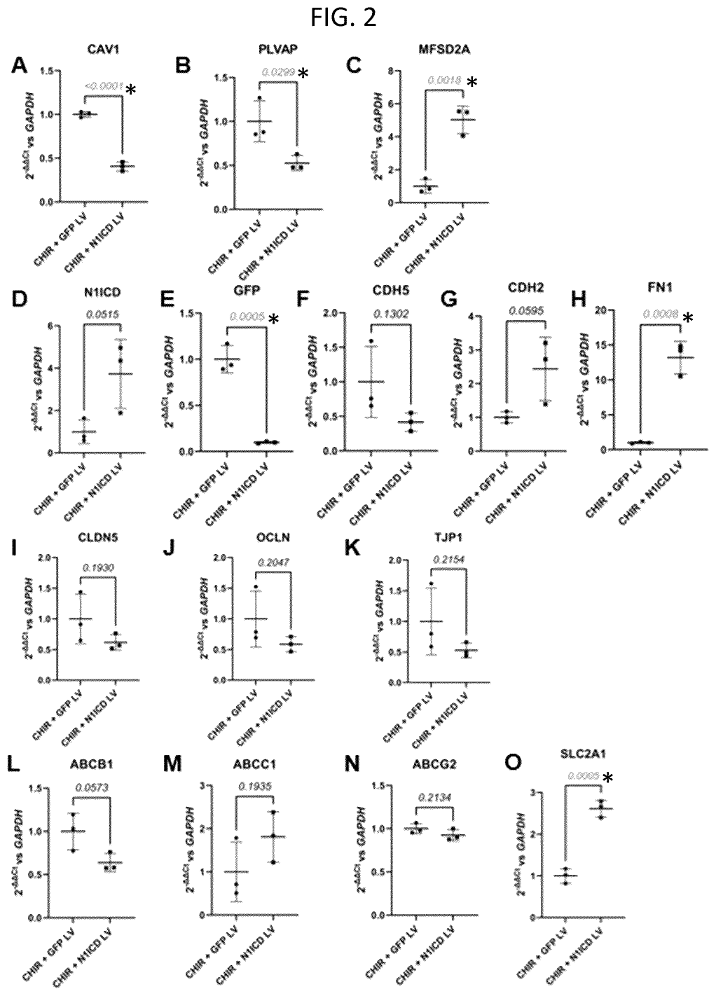

Unedited hPSC-derived EPCs (D5) with high post-MACS purity validated by flow cytometry were cultured on collagen IV-coated plates at approximately 4×10⁴ cells/cm² in hECSR medium supplemented with 4 μM CHIR (Tocris). For characterization by flow cytometry, RT-qPCR, or western blotting, EPCs were seeded in 6-well plates. For characterization by immunocytochemistry, EPCs were seeded in 48-well plates. Medium was changed every other day until D11. On D7, EPCs were transduced with 5.26 μL/cm² N1ICD LV (50 μL/well in a 6-well plate, 5 L/well in a 48-well plate) or 0.66 μL/cm² GFP LV negative control (6.25 μL/well in a 6-well plate, 0.625 μL/well in a 48-well plate). On D11, cells were isolated for various downstream assays. Flow cytometry was used to determine expression of GFP, CD31, and caveolin-1 or PLVAP (Table 2). Expression of several genes was assayed by RT-qPCR (FIG. 2, Table 1). Western blotting was used to quantify expression of Notch1 (including full length protein and intracellular domain [N1ICD]), phospho-JNK, total JNK, and β-actin (Table 2). Cells were stained for GFP, caveolin-1, PLVAP, GLUT-1, and PECAM-1 and imaged with immunofluorescence microscopy (Table 2).

TABLE 2
Antibodies

| | Species/ | Manufacturer, clone | | | |
|---|---|---|---|---|---|
| Target | Isotype | (product number), RRID | Fluorophore | App* | Dilution |
| Caveolin-1 | Rabbit | Cell Signaling Technology | Unconjugated | ICC | 1:500 |
| | polyclonal | (3238), RRID: | | | |
| | | AB_2072166 | | | |
| Caveolin-1 | Rabbit | Cell Signaling Technology | Unconjugated | TEM | 1:100 |
| | monoclonal | (3267) | | | |
| | | RRID: AB_2275453 | | | |
| Caveolin-1 | Host unknown | Miltenyi Biotec REAL686 | PE | FC | 1:50 |
| | monoclonal | (130-129-282), RRID: | | | |
| | | AB_2904649 | | | |
| CD31 | Rabbit | Lab Vision (RB-10333-P), | Unconjugated | ICC | 1:100 |
| (PECAM-1) | polyclonal | RRID: AB_720502 | | | |
| CD31 | Mouse IgG1 | Miltenyi Biotec AC128 | APC | FC | 1:50 |
| (PECAM-1) | | (130-119-891), RRID: | | | |
| | | AB_2784124 | | | |
| CD34 | Mouse IgG2a | Miltenyi Biotec AC136 | FITC | FC | 1:50 |
| | | (130-113-178), RRID: | | | |
| | | AB_2726005 | | | |
| CD144 (VE- | Recomb. human | Miltenyi Biotec REA199 | APC | FC | 1:50 |
| cadherin) | IgG1 | (130-125-985), RRID: | | | |
| | | AB_2857821 | | | |
| CD144 (VE- | Recomb human | Miltenyi Biotec REA199 | FITC | FC | 1:50- |
| cadherin) | IgG1 | (130-123-688), RRID: | | | 1:100 |
| | | AB_2819510 | | | |
| GFP | Mouse IgG2a | Santa Cruz, B-2 (sc-9996), | Unconjugated | ICC | 1:100 |
| | | RRID: AB_627695 | | | |
| GLUT-1 | Mouse IgG2a | Invitrogen SPM498 | Unconjugated | ICC | 1:200 |
| | | (MA5-11315), RRID: | | | |
| | | AB_10979643 | | | |
| Isotype | Mouse IgG1 | R&D Systems 11711 | APC | FC | 1:50 |
| control | | (IC002A), | | | |
| | | RRID: AB_357239 | | | |
| Isotype | Mouse IgG1, κ | BioLegend MOPC-21 | FITC | FC | 1:50 |
| control | | (400108), RRID: | | | |
| | | AB_326429 | | | |
| Notch1 | Rabbit IgG | Cell Signaling Technology, | Unconjugated | WB | 1:500 |
| | | D1E11 (3608), RRID: | | | |
| | | AB_2153354 | | | |
| PLVAP | Rabbit | Prestige Antibodies | Unconjugated | ICC | 1:200 |
| | polyclonal | (HPA002279), RRID: | | | |
| | | AB_1079636 | | | |
| β-actin | Rabbit IgG | Cell Signaling Technology, | Unconjugated | WB | 1:1000 |
| | | 13E5 (4970), RRID: | | | |
| | | AB_2223172 | | | |
| Rabbit IgG | Goat polyclonal | LI-COR, (925-58071), | IRDye 680RD | WB | 1:5000 |
| | | RRID: AB_10956166 | | | |
| Mouse IgG2a | Goat polyclonal | Invitrogen (A-21131), | Alexa Fluor | ICC | 1:200 |
| | | RRID: AB_141618 | 488 | | |
| Rabbit IgG | Goat polyclonal | Invitrogen (A-11008), | Alexa Fluor | ICC | 1:200 |
| | | RRID: AB_143165 | 488 | | |
| Mouse IgG | Goat polyclonal | Invitrogen (A-21422), | Alexa Fluor | ICC | 1:200 |
| | | RRID: AB_141822 | 555 | | |
| Rabbit IgG | Goat polyclonal | Invitrogen (A-11012), | Alexa Fluor | ICC | 1:200 |
| | | RRID: AB_2534079 | 594 | | |
| Mouse IgG2a | Goat polyclonal | Invitrogen (A-21135), | Alexa Fluor | ICC | 1:200 |
| | | RRID: AB_2535774 | 594 | | |
| Rabbit IgG | Goat polyclonal | Invitrogen (A-21245), | Alexa Fluor | ICC | 1:200 |
| | | RRID: AB_2535813 | 647 | | |
| Mouse IgG1 | Goat polyclonal | Invitrogen (A-21240), | Alexa Fluor | ICC | 1:200 |
| | | RRID: AB_141658 | 647 | | |
| Mouse IgG | Goat polyclonal | Invitrogen (A-21235), | Alexa Fluor | ICC | 1:200 |
| | | RRID: AB_2535804 | 647 | | |
| Rabbit IgG | Goat polyclonal | Nanoprobes (#2003), | Nanogold ® | TEM | 1:50 |
| | | RRID: AB_2687591 | | | |

*Application: FC, flow cytometry; ICC, immunocytochemistry; WB, western blotting; TEM, transmission electron microscopy

Generation of Doxycycline-Inducible N1ICD-Overexpressing hPSCs

N1ICD and preceding Kozak sequence were PCR amplified using Q5 High-Fidelity 2× Master Mix (New England Biolabs) from pWPI-N1ICD with primers containing NheI and AgeI restriction site overhangs (Table 1). The amplification product was run on a 1% agarose gel. The band corresponding to the N1ICD amplicon with NheI and AgeI overhangs was cut out of the gel and DNA was purified using the Monarch DNA Gel Extraction Kit (New England Biolabs). Plasmid PB-TRE-ETV2 was digested using NheI-HF and AgeI-HF (New England Biolabs) to separate the ETV2 cassette from the plasmid backbone containing a doxycycline-inducible TRE3G promoter, an EF-1α core promoter followed by a Tet-On 3G cassette, a hygromycin resistance cassette, and 3′ and 5′ piggyBac inverted repeats. The backbone also contained an ampicillin resistance cassette outside the piggyBac inverted repeats for cloning purposes. The restriction digest fragments of PB-TRE-ETV2 were separated by gel electrophoresis on a 1% agarose gel into ˜8 kb backbone and ˜1 kb ETV2 insert. The band corresponding to the backbone was excised and DNA was purified using the Monarch DNA Gel Extraction Kit (New England Biolabs). Additionally, the N1ICD amplicon with NheI and AgeI overhangs was similarly digested with NheI-HF and AgeI-HF to create sticky ends. The digested N1ICD amplicon with restriction overhangs was column washed using the DNA Clean and Concentrator-5 kit (Zymo Research, Irvine, CA). Sticky-end ligation of the N1ICD PCR product with restriction overhangs (˜2.5 kb) and PB-TRE-ETV2 plasmid backbone (˜8 kb) was performed with the T4 DNA Ligase (Thermo Scientific, Waltham, MA). The resulting ˜10.5 kb plasmid (PB-TRE-N1ICD) was transformed into NEB Stable Competent E. coli (New England Biolabs). Single ampicillin-resistant colonies were picked, expanded, and plasmid DNA was purified with the ZymoPURE Plasmid Miniprep Kit (Zymo Research). The resulting PB-TRE-N1ICD plasmids were digested with NheI-HF and AgeI-HF (New England Biolabs), and the resulting restriction fragments were run on a 1% agarose gel to verify presence of an 8 kb backbone and 2.5 kb insert (N1ICD) and absence of a 1 kb insert (ETV2). Clones with appropriate restriction fragments were Sanger sequenced with 10 sequencing primers (Table 1) to verify that the N1ICD sequenced matched that in pWPI-N1ICD.

Unedited IMR90-4 hPSCs were reverse-transfected with a Super PiggyBac Transposase Expression Vector (PB210PA-1 [System Biosciences]) and the doxycycline-inducible N1ICD overexpressing transposon plasmid (PB-TRE-N1ICD) using the TransIT-LT1 Transfection Reagent (Mirus Bio, Madison, WI) according to the manufacturer's protocol. Briefly, 0.5 mL mTeSR1 medium (STEMCELL Technologies) was added to a well in a Matrigel-coated 6-well plate. In a sterile tube, 400 μL Opti-MEM (Life Technologies) was supplemented with 12 μL TransIT-LT1 Transfection Reagent and 4 μg plasmid DNA (5:2 ratio of PB-TRE-N1ICD [2857.1 ng] to PB210 [1142.9 ng]), mixed and incubated at room temperature for 15 min. During incubation, hPSCs were singularized for 10 min with Accutase (Innovative Cell Technologies), quenched in 4× volume of mTeSR1 medium and centrifuged for 5 min at 200×g. During the centrifugation, the plasmid and transfection reagent mixture was added to the well containing mTeSR1 medium and incubated at room temperature for the remainder of the procedure. Accutased hPSCs were counted and resuspended in mTeSR1 medium to a final concentration of 2×10⁶ cells/mL. Cells were transferred to the well plate containing TransIT-LT1: DNA complexes. The medium was supplemented with 10 μM ROCK inhibitor Y-27632 (Tocris) and 1×Antibiotic-Antimycotic (Life Technologies), and cells were incubated at 37° C., 5% CO₂. 24 h later, medium was replaced with mTeSR1 medium. Transfected hPSCs were cultured with daily mTeSR1 medium changes until 60-70% confluent, then cells were passaged to a new Matrigel coated plate. After the cells reached ˜20% confluency, media was replaced with mTeSR Plus medium (STEMCELL Technologies) supplemented with 50 μg/mL hygromycin B in PBS (Invitrogen, Carlsbad, CA). Media was replaced daily with mTeSR Plus medium supplemented with 50 μg/mL hygromycin. If cell density appeared to decline on consecutive days, hygromycin was omitted and mTeSR Plus was supplemented with 10 μM Y-27632. Cells were passaged with Versene in mTeSR Plus medium without hygromycin at a split ratio of 1:6. Hygromycin selection of transfected hPSCs was performed for a total of three passages, after which 70-80% confluent wells of the heterogeneous population of PB-TRE-N1ICD hPSCs were cryopreserved in hPSC mTeSR1 freezing medium (60% mTeSR1, 30% FBS, 10% DMSO). N1ICD and HEYL expression in heterogeneous PB-TRE-N1ICD hPSCs treated with 1 μg/mL doxycycline hyclate (Thermo Scientific) in sterile DPBS or 1:1000 diluted sterile DPBS was determined by RT-qPCR.

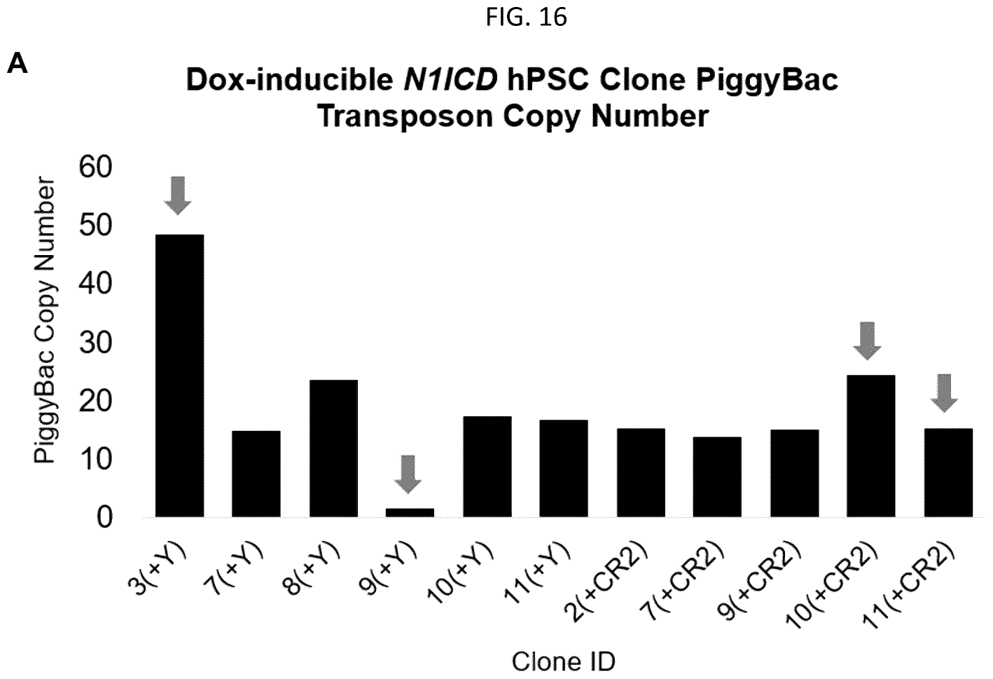

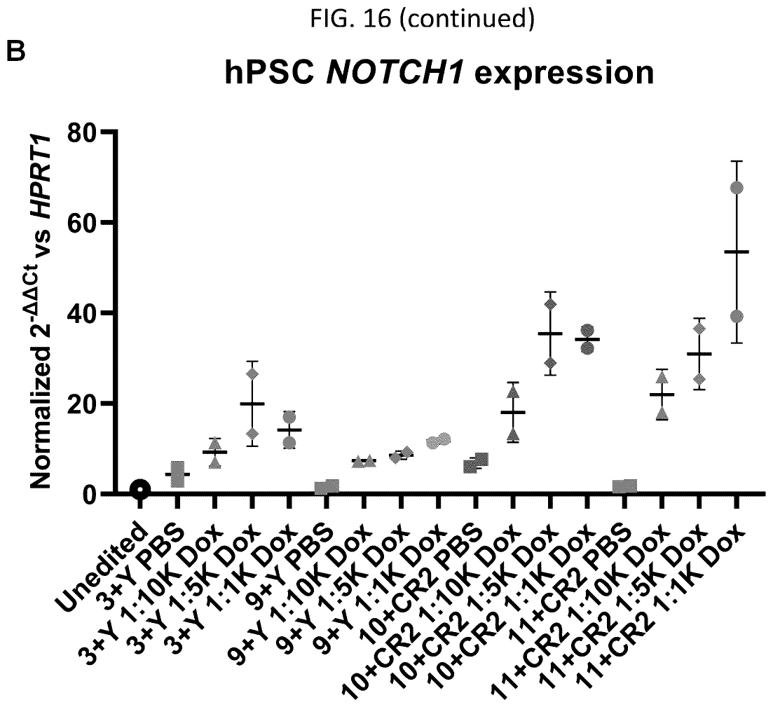

Next, we selected PB-TRE-NICD hPSC clones with specific copy numbers of the integrated PB-TRE-N1ICD transposon. Three 10 cm Matrigel-coated cell culture dishes (6 mL DMEM/F-12 supplemented with Matrigel [see “hPSC Maintenance” for details] added to each ˜57 cm² dish) were prepared and incubated at 37° C., 5% CO₂ for a minimum of 1 h before use. One well of ˜70% confluent IMR90-4 PB-TRE-N1ICD hPSCs was dissociated with Accutase for 9 min and quenched through a 40 μm cell strainer (Beckton Dickinson) in 4× volume of mTeSR Plus. Cell counts were quantified by hemacytometer. 2×10², 2×10³, and 1×10⁴ cells were separately suspended in 8 mL each of mTeSR Plus supplemented with 1×CloneR2 (STEMCELL Technologies), added to the Matrigel-coated dishes, agitated horizontally and side-to-side, and incubated at 37° C., 5% CO₂. Medium was changed daily with mTeSR Plus supplemented with 1×CloneR2 until colonies became visible. Afterwards, medium was changed daily with mTeSR Plus. After ˜7 days, when colonies had grown but were not touching, 12 colonies from 2×10³ cells/dish and 11 colonies from 2×10² cells/dish were picked. Briefly, Matrigel-coated 12-well plates were pre-filled with 1 mL/well mTeSR Plus supplemented with 1×Antibiotic-Antimycotic. Additionally, plates receiving colonies picked from 2×10³ cells/dish or 2×10² cells/dish were supplemented 1×CloneR2 or 10 μM Y-27632, respectively. Individual colonies were visualized with the EVOS XL Core Imaging System (Life Technologies), manually dislodged and aspirated with a p200 pipette tip, transferred into a separate well in the Matrigel-coated 12-well plate, and incubated at 37° C., 5% CO₂. Medium was changed daily with mTeSR Plus supplemented with 1×CloneR2 or 10 μM Y-27632 until colonies had attached and begun to proliferate. After this, medium was changed daily with mTeSR Plus. When the center of a picked colony became opaque under a brightfield microscope or increased cell death was observed, the colony was passaged with Versene to one well in a Matrigel-coated 6-well plate in mTeSR Plus supplemented with 10 μM Y-27632. Subsequent Versene passages were performed without addition of 10 μM Y-27632. In total, 11 clonal hPSC populations were isolated, gDNA was extracted using the Monarch Genomic DNA Purification Kit (New England Biolabs), and PB-TRE-N1ICD transposon copy number in each clone was quantified using PiggyBac qPCR Copy Number Kit (System Biosciences). Cells were cryopreserved in hPSC mTeSR1 freezing medium. Four clones with variable transposon copy numbers were thawed and cultured in E8 medium supplemented with 1:1000 diluted sterile DPBS or different doxycycline concentrations (1 μg/mL, 200 ng/mL, and 100 ng/mL), and NOTCH1 expression was quantified by RT-qPCR (FIG. 16B). All 4 clonal PB-TRE-N1ICD hPSC lines (3[+Y], 9 [+Y], 10 [+CR2], and 11[+CR2]) were sent for G-banded karyotyping (WiCell, Madison, WI) and determined to have normal karyotypes.

Doxycycline-Induced N1ICD Overexpression in Gene-Edited PB-TRE-N1ICD hPSC-EPCs

Gene-edited hPSC-derived EPCs (D5) with high post-MACS purity validated by flow cytometry were cultured on collagen IV-coated plates at approximately 4×10⁴ cells/cm² in hECSR medium supplemented with various antibiotics, with media changes every other day. From D5 until D11, hECSR medium was supplemented with 4 μM CHIR (Tocris) and 10 μg/mL hygromycin B (Invitrogen). From D11 until D15, hECSR medium was supplemented with 4 μM CHIR and either 1:1000 DPBS or 1 μg/mL doxycycline hyclate (Thermo Scientific) in DPBS. On D15, cells were collected for RT-qPCR quantification of differential gene expression between doxycycline and DPBS-treated gene-edited hPSC-EPCs.

### Immunocytochemistry

Cells were washed once with DPBS and fixed with ice-cold methanol for 15 min. Cells were washed once with DPBS and blocked in DPBS supplemented with 10% goat serum (Life Technologies) for 1 h at room temperature on a rocking platform. Cells were washed once with DPBS. Primary antibodies (Table 2) diluted in DPBS supplemented with 10% goat serum were added to cells and incubated at 4° C. overnight (18-24 h) on a rocking platform. The following day, cells were washed 2 times with DPBS. Secondary antibodies (Table 2) diluted in DPBS supplemented with 10% goat serum were added to cells and incubated for 1 h at room temperature on a rocking platform, protected from light. Cells were washed once with DPBS and incubated for 5 min in DPBS supplemented with 4 μM Hoechst 33342 (Life Technologies). Images were acquired using an Eclipse Ti2-E epifluorescence microscope (Nikon, Tokyo, Japan) with a 10× or 20× objective. Images were analyzed using FIJI.

### Flow Cytometry

To validate expression of CD31 and CD34 in differentiated hPSC-EPCs (FIG. 3C), flow cytometry samples were prepared on D5 of differentiation. hPSC-EPCs were incubated for 15 min at 37° C., 5% CO₂ with Accutase, triturated, and quenched through a 40 μm cell strainer into 4× the volume of DMEM (Life Technologies) supplemented with 10% FBS (R&D Systems). Cell count was quantified using a hemocytometer and cells were centrifuged at 200×g for 5 mins. Cells were resuspended in MACS buffer at a concentration of 100 μL/10⁶ cells. Two aliquots of 10⁶ cells each were aliquoted pre-MACS and kept at 4° C. during MACS. (4-5)×10⁵ cells were aliquoted post-MACS. Pre- and post-MACS cell suspensions were supplemented with CD31-APC and CD34-FITC antibodies or corresponding APC and FITC isotype control antibodies (Table 2) at a 1:50 dilution and incubated at 4° C. for 20 min, protected from light. Cells were washed with 2 mL MACS buffer and centrifuged at 200×g for 5 min. Pellets were resuspended in 500 μL each of 4% paraformaldehyde (PFA, Electron Microscopy Sciences, Hatfield, PA) in DPBS and incubated at room temperature for 15 min, protected from light. Cells were centrifuged at 200×g for 5 min and pellets were resuspended in 400 μL MACS buffer. Samples were run on an Attune NxT V6 flow cytometer (Thermo Fisher) with excitation at 488 nm and 633 nm. FITC emission was detected with a 530/30 filter and APC emission was detected with a 670/14 filter. Data were analyzed with Flow Jo software (BD Biosciences, San Jose, CA).

To determine the transduction efficiency of the GFP and N1ICD lentiviruses and measure CD31 expression in transduced unedited hPSC-EPCs (FIG. 11C,D), cells were isolated on D11 to prepare flow cytometry samples. Cells were initially prepared in a similar fashion to the hPSC-EPCs described above. Pellets of transduced cells were resuspended in MACS buffer (100 L/10⁶ cells) supplemented with CD31-APC or APC isotype control antibodies at a 1:50 dilution. Pellets of untransduced cells were resuspended in MACS buffer supplemented with CD31-APC. Cells were incubated at 4° C. for 20 min, protected from light. Cells were washed with 2 mL MACS buffer and centrifuged at 200×g for 5 min. Pellets were resuspended in 500 μL each of 1% PFA in DPBS and incubated 15 min at room temperature, protected from light. Cells were centrifuged at 200×g for 5 min and pellets were resuspended in 400 μL MACS buffer. Samples were run on an Attune NxT V6 flow cytometer with excitation at 488 nm and 633 nm. GFP emission was detected with a 530/30 filter and APC emission was detected with a 670/14 filter. Data were analyzed with Flow Jo software.

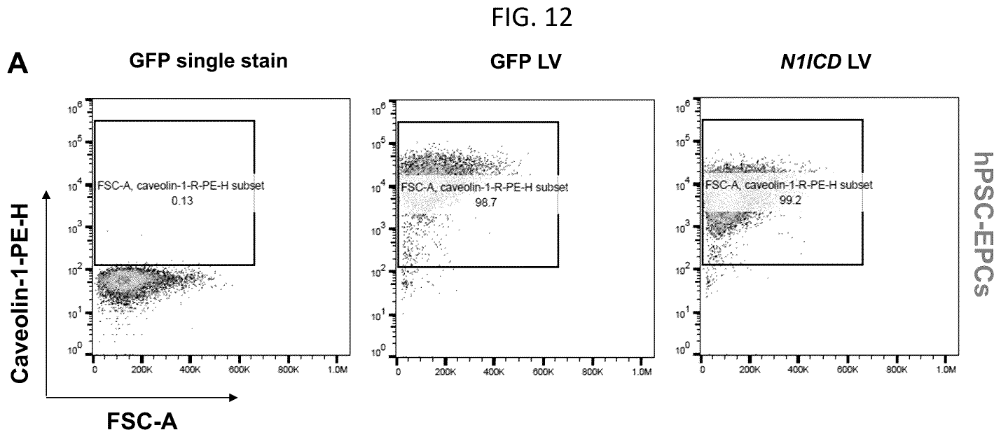

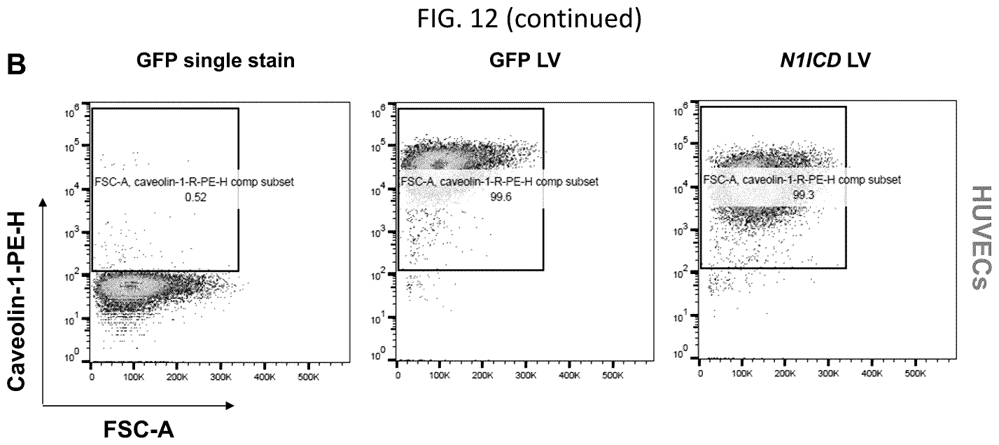

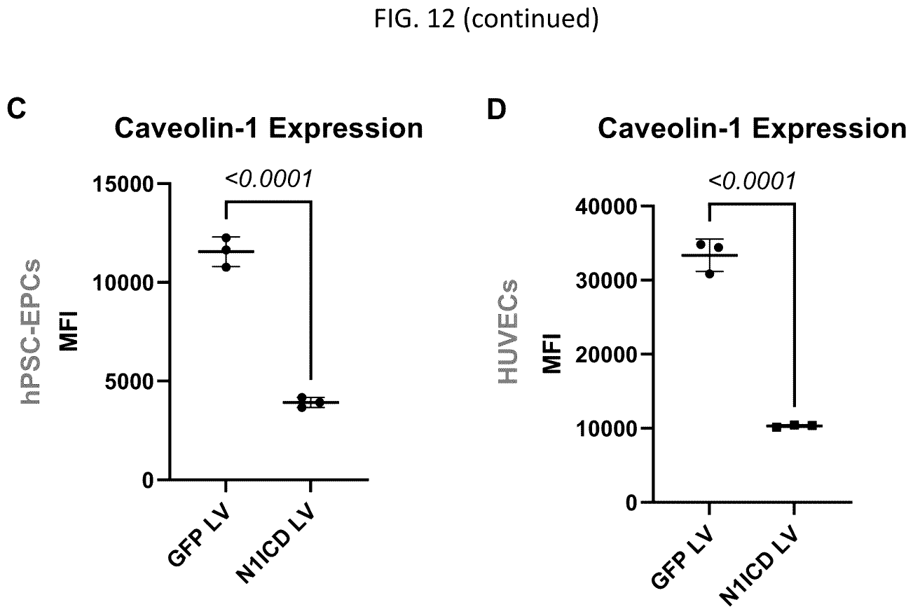

To determine caveolin-1 and CD31 expression in GFP LV or N1ICD LV transduced hPSC-EPCs or HUVECs (FIG. 12,13), hPSC-ECs were isolated at D11 and HUVECs were isolated at 6 days following transduction, respectively. Samples also included unstained and fluorescence minus one (FMO) controls. Cells were dissociated with either Accutase or Trypsin-EDTA, respectively, as previously stated, strained, and resuspended in MACS buffer (100 μL/10⁶ cells). Cell suspensions were supplemented with 1:50 diluted CD31-APC and incubated at 4° C. for 20 min, protected from light. Cells were washed with 2 mL MACS buffer and centrifuged at 200×g for 5 min. Pellets were resuspended in 500 μL each of 4% PFA in DPBS and incubated 15 min at room temperature, protected from light. Cells were centrifuged at 200×g for 5 min, and pellets were resuspended in 500 μL 0.1% Triton X-100 (Sigma Aldrich) in MACS buffer and incubated 30 min at room temperature, protected from light. Cells were washed with 2 mL MACS buffer and centrifuged at 200×g for 5 min. Cells were then resuspended in 400 μL MACS buffer supplemented with 1:50 diluted caveolin-1-PE antibody (Table 2) and incubated at 4° C. overnight on a rocking platform, protected from light. The following day, cells were washed twice with 2 mL MACS buffer and centrifuged at 200×g for 5 min. Cells were resuspended in 400 μL MACS buffer. Samples were run on an Attune NxT V6 flow cytometer with excitation at 488 nm, 561 nm, and 633 nm. GFP emission was detected with a 530/30 filter, PE emission was detected with a 585/16 filter, and APC emission was detected with a 670/14 filter. Data were analyzed with Flow Jo software.

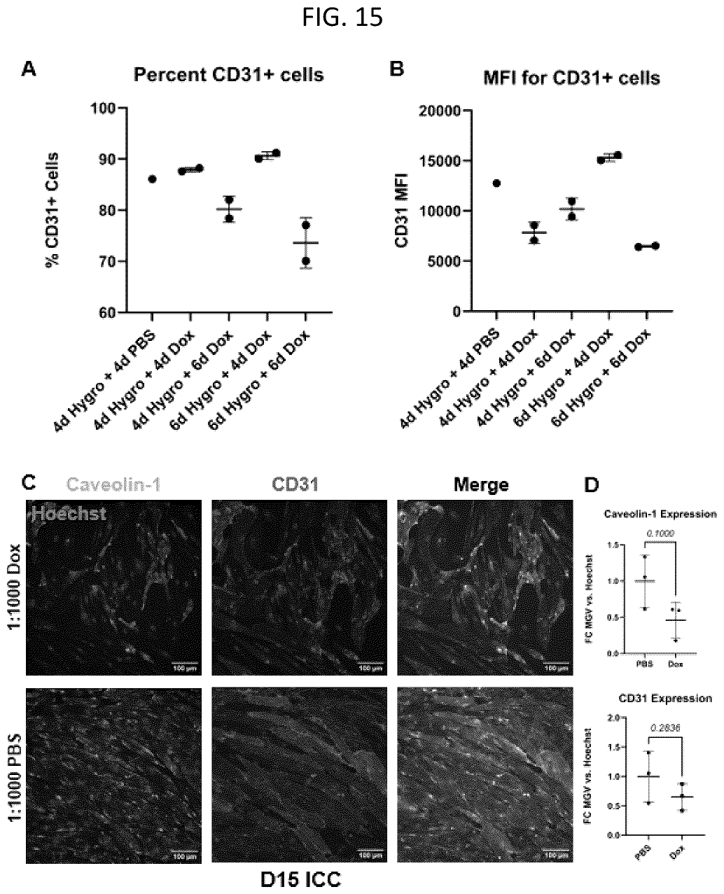

To determine CD31 expression in edited hPSC-EPCs treated with doxycycline vs. DPBS (FIG. 15A,B), hPSC-ECs were isolated at day 13, 15, or 17, dissociated with Accutase as previously stated, quenched in DMEM supplemented with 10% FBS through a cell strainer, centrifuged, and resuspended in MACS buffer (100 μL/10⁶ cells) supplemented with CD31-APC or APC isotype control antibodies at a 1:50 dilution. Cells were incubated at 4° C. for 20 min, protected from light. Cells were washed with 2 mL MACS buffer and centrifuged at 200×g for 5 min. Pellets were resuspended in 500 μL each of 4% PFA in DPBS and incubated 15 min at room temperature, protected from light. Cells were centrifuged at 200×g for 5 min and pellets were resuspended in 400 μL MACS buffer. Samples were run on an Attune NxT V6 flow cytometer with excitation at 633 nm. APC emission was detected with a 670/14 filter. Data were analyzed with Flow Jo software.

### Fluorescence-Activated Cell Sorting (FACS)

Gene-edited PB-TRE-N1ICD hPSC-EPCs were cultured in a 6-well plate with or without doxycycline as described above (n=3* replicates for doxycycline supplemented condition, n=4 replicates for PBS supplemented condition). On D15, cells were isolated with Accutase (incubated 15 mins at 37° C., 1 mL/well), triturated, and quenched through a 40 μm cell strainer into 4× the volume of DMEM (Life Technologies) supplemented with 10% FBS (R&D Systems). An additional well of gene-edited EPCs cultured with PBS was reserved as an isotype control. Unedited hPSCs maintained in E8 medium and unedited hPSC-EPCs cultured for 6 days in hECSR supplemented with 4 μM CHIR were used as an additional negative and positive control, respectively, for FACS. Cell count was quantified using a hemocytometer and cells were centrifuged at 200×g for 5 min. Cells were resuspended in MACS buffer at a concentration of 100 μL/10⁶ cells. Cells suspensions were supplemented with CD144-APC or APC-conjugated isotype control antibodies (Table 2) at a 1:50 dilution for at 4° C. for 20 min, protected from light. Cells were washed with 2 mL MACS buffer and centrifuged at 200×g for 5 min. Pellets were resuspended in 400 μL each of MACS buffer supplemented with 1 μg/mL DAPI (Invitrogen) and incubated at 4° C., protected from light.

Cells were sorted using the BD FACSAria III Cell Sorter (BD Biosciences). Gates for high or low CD144-expressing cells were determined based on two negative controls (gene-edited PB-TRE-N1ICD hPSC-derived ECs stained with APC isotype control antibody or unedited hPSCs stained with CD144-APC antibody, both negative for CD144) and one positive control (unedited hPSC-EPCs cultured with CHIR, CD144 high). After establishing gates, live gene-edited PB-TRE-N1ICD hPSC-ECs treated with or without doxycycline were sorted into tubes containing 500 μL MACS buffer and stored at 4° C.

*Note: 4 biological replicates were supplemented with doxycycline, but 2 replicates were unintentionally combined during FACS sample preparation.

### RT-qPCR

RNA extraction was performed using the Direct-zol RNA Miniprep Kit (Zymo Research). Cells were lysed with 300-600 μL TRIzol reagent (Invitrogen, Waltham, MA) depending on the estimated cell count per sample. Cell lysates were combined with an equal volume of 100% ethanol and transferred to Zymo-Spin IICR spin columns. Columns were incubated with Rnase-free DNase I (Qiagen) to eliminate residual gDNA. Columns were washed with RNA Wash Buffer and Direct-zol RNA Pre-Wash according to Zymo RNA Miniprep Kit protocols. RNA was eluted with RNase-free water and concentration was determined using a NanoDrop 2000 spectrophotometer (Thermo Scientific). 250-1000 ng of RNA was reverse transcribed for 1 h at 37° C. using the OmniScript RT Kit (Qiagen). 1 U/μL RNaseOUT (Life Technologies) was included in the reverse-transcription reactions. Reaction products were diluted to 10 ng/μL. 20 μL qPCR reactions were carried out with 10 ng cDNA and 500 nM each forward and reverse primers (Integrated DNA Technologies, Coralville, IA [Table 1]) using PowerUp SYBR Green Master Mix (Life Technologies) or 1×Taqman Gene Expression Assay probes (Thermo Scientific [Table 1]) using Taqman Fast Advanced Master Mix (Thermo Scientific). Reactions were run on an AriaMx Real-Time PCR System (Agilent Technologies, Santa Clara, CA) using the thermal cycling program corresponding to either IDT primers or Taqman probes. An annealing temperature of 60° C. was used for all reactions.

### Western Blotting

Cells were lysed with radioimmunoprecipitation assay (RIPA) buffer (Rockland Immunochemicals, Pottstown, PA) supplemented with Pierce Protease and Phosphatase Inhibitor (Thermo Scientific) and centrifuged at 4° C. for 5 min, 16,000×g. Supernatants were collected, transferred to new tubes, and stored at −80° C. Protein concentration was quantified using the Pierce BCA Protein Assay Kit. For each sample, 6.5 μg of protein was diluted to equal volume with water, mixed with sample buffer, and heated to 95° C. for 5 min. Samples were then run on 4-12% Tris-glycine gels and transferred to nitrocellulose membranes. Membranes were blocked for 1 h in tris-buffered saline with 0.1% Tween-20 (TBST) supplemented with 5% non-fat dry milk. Primary antibodies (Table 2) were diluted in TBST supplemented with 5% non-fat dry milk, added to membranes, and incubated overnight (16-24 h) at 4° C. on a rocking platform. Membranes were washed 5 times with TBST. Secondary antibodies (Table 2) were diluted in TBST supplemented with 5% non-fat dry milk, added to membranes, and incubated for 1 h at room temperature on a rocking platform, protected from light. Membranes were washed 5 times with TBST and imaged using an Odyssey 9120 (LI-COR, Lincoln, NE). The same membrane was blotted for both Notch1 and β-actin; however, the membrane was initially blotted only for Notch1 and following imaging, the membrane was re-blotted for β-actin. Band intensities were quantified using Image Studio software (LI-COR).

### Fluorescent Albumin Accumulation Assay

Fixable, Alexa Fluor 647-conjugated bovine serum albumin (BSA) (Invitrogen) was used as a substrate to estimate total fluid-phase endocytosis. D15 cultures of gene-edited PB-TRE-N1ICD hPSC-EPCs treated with 4 μM CHIR with or without 1 μg/mL doxycycline were pre-treated for 30 min at 37° C. with or without various endocytic pathway inhibitors (3 mM methyl-β-cyclodextrin [MβCD, Sigma-Aldrich], 20 UM chlorpromazine [CPZ, Sigma-Aldrich], or 2 μM rottlerin [Tocris]). Similarly, D6 cultures of HUVECs treated with GFP or N1ICD LV were pre-treated with or without 3 mM MβCD for 30 min at 37° C. Cells were washed once with sterile DPBS and then incubated in hECSR (for hPSC-EPCs) supplemented with 5 μg/mL BSA or 1:1000 diluted DPBS, 1:100 diluted CD144-FITC or FITC isotype control antibody, and 4 μM Hoechst 33342 (Life Technologies) or EGM-2 media (for HUVECs) supplemented with 5 μg/mL BSA or 1:1000 diluted DPBS and 4 μM Hoechst 33342 at 4° C. or 37° C. for 2 h on a rotating platform at 30 rpm, protected from light. Cells were then washed 3 times with 4° C. sterile DPBS to stop the assay and wash away any extracellular BSA. Internalization of fluorescent BSA was visualized using an Eclipse Ti2-E epifluorescence microscope (Nikon, Tokyo, Japan) with a 20× objective.

Flow cytometry was used to quantify accumulated BSA fluorescence within cells. Individual well replicates were dissociated with Accutase (for edited 10(+CR2) hPSC-EPCs, 1 mL/well for 15 min at 37° C.) or 0.25% Trypsin-EDTA (for HUVECs, 0.5 mL/well for 10 min at 37° C.), quenched in hECSR (hPSC-EPCs) or EGM-2 (HUVECs) through a 40 μm cell strainer and centrifuged. Pellets were resuspended in 500 μL each of 4% PFA in DPBS and incubated 15 min at room temperature, protected from light. Cells were centrifuged at 200×g for 5 min and pellets were resuspended in 400 μL MACS buffer. For 3(+Y), 9(+Y), and 11 (+CR2) edited hPSC-EPCs, cells were dissociated with 300 μL/well of Accutase and quenched directly into 1.2 mL hECSR without straining in a 96-well V-bottom deep well plate (Corning). The plate was centrifuged at 300×g for 5 min at 4° C., and pellets were resuspended in 400 μL MACS buffer without a prior fixation step. All samples were run on an Attune NxT V6 flow cytometer with excitation at 488 nm and/or at 633 nm. FITC emission was detected with a 530/30 filter and AlexaFluor 647 emission was detected with a 670/14 filter. Data were analyzed with Flow Jo software.

### Caveolin-1 Immunogold Labeling and Transmission Electron Microscopy

Gene-edited PB-TRE-N1ICD hPSC-EPCs were cultured on sterile 15 mm round cover glass (CELLTREAT Scientific Products, Pepperell, MA) coated with 10 μg/mL collagen-IV within a 12-well tissue culture plate (Corning). Cells were seeded at 4×10⁴ cells/cm² in hECSR supplemented with 4 μM CHIR, 1:1000 diluted DPBS, and 1×antibiotic-antimycotic (Life Technologies). Cells were cultured with media changes every other day until D11. On D11, media was changed with hECSR supplemented with 4 μM CHIR, 1 μg/mL doxycycline or 1:1000 diluted DPBS, and 1×antibiotic-antimycotic. This media was changed every other day until D15.

On D15, cells were washed once with DPBS. N=4 wells each treated with doxycycline or DPBS were fixed with 4% PFA (Electron Microscopy Sciences)+0.1% glutaraldehyde (GA, Electron Microscopy Sciences) in 0.1M Sorensen's phosphate buffer (PB) (for caveolin-1 immunogold labeling) or 2% PFA+2.5% GA in 0.1M PB (for ultrastructural imaging) for 15 min at room temperature. Cells were washed with DPBS. Cells fixed with 2% PFA+2.5% GA were stored at 4° C. in DPBS until proceeding with downstream processing for TEM.

Cells fixed with 4% PFA+0.1% GA (for caveolin-1 immunogold labeling) and were permeabilized with 0.1% saponin from Quillaja bark (Sigma-Aldrich) in DPBS for 10 min at room temperature. Cells were washed with DPBS and blocked in 10% goat serum in DPBS for 1 h, rocking at room temperature. Cells were washed with DPBS and incubated overnight rocking at 4° C. in 10% goat serum supplemented with α-caveolin-1 primary antibody (Table 2). Cells were washed twice with DPBS, and incubated rocking at 4° C. in 10% goat serum supplemented with Nanogold-conjugated secondary antibody (Table 2, Nanoprobes, Yaphank, NY). Cells were washed in DPBS. Cells were post-fixed with 1% GA in phosphate buffer for 30 min at room temperature. The HQ Silver kit (Nanoprobes) was used to silver enhance samples for 7 min at room temperature, protected from light. All samples (both ultrastructure imaging and caveolin-1 immunogold staining) were embedded in epoxy resin and sectioned parallel to the cell culture surface with a microtome before mounting on TEM grids. Sample sections were subsequently treated with osmium tetroxide (Electron Microscopy Sciences) for 20 min. Samples for ultrastructure imaging were contrast stained with uranyl acetate for 15 min followed by lead citrate for 10 min. Samples with caveolin-1 immunogold labeling were contrasted with a lead citrate and uranyl acetate solution for 30 seconds. Grids were imaged using a FEI CM120 transmission electron microscope (Philips, Amsterdam, Netherlands). Images were analyzed using FIJI by a researcher who was blinded to the treatment condition. Number of vesicles, vesicle diameter, and total cell area were measured manually, while Nanogold area was determined via thresholding.

### Transendothelial Electrical Resistance (TEER)

Transwell inserts (6.5 mm diameter with 0.4 μm pore polyester membrane) (Corning) were coated with 50 μl of a collagen-IV (400 μg/mL) and fibronectin (100 μg/mL) solution in sterile water and incubated at 37° C. for a minimum of 4 h before seeding cells. Gene-edited PB-TRE-N1ICD hPSC-EPCs (clone 10 [+CR2]) were seeded on D5 at a density of 10⁵ cells/cm² onto the Transwell membrane in hECSR medium supplemented with 4 μM CHIR and 1:1000 diluted DPBS. Medium volumes were 100 μL for the apical chamber and 600 μL for the basolateral chamber. Medium was replaced every other day. Starting the day after seeding, TEER was measured daily for 10 days with an EVOM2 epithelial voltohmeter with STX2 chopstick electrodes (World Precision Instruments, Sarasota, FL). TEER values were calculated by subtracting the resistance of a collagen-IV/fibronectin-coated Transwell insert without cells and multiplying by the Transwell surface area (0.33 cm²).

### Bulk RNA-Sequencing and Analysis

Ten PBS or doxycycline-treated gene-edited PB-TRE-N1ICD hPSC-derived endothelial cell subpopulations obtained via FACS were centrifuged at 200×g for 5 min. Pellets were resuspended TRIzol reagent (Invitrogen) for extraction of total RNA. RNA concentrations were quantified using the Qubit 4 Fluorometer (Invitrogen) and Qubit RNA High Sensitivity Assay Kit (Invitrogen). Purified RNA samples (≥200 ng) were sent to Novogene Corporation (Sacramento, CA) for library preparation and mRNA sequencing, with approximately 40-60 million paired end reads obtained per sample. FASTQ files were uploaded to the Galaxy⁴³ web platform and processed to generate a raw counts matrix using the publicly available bioinformatics server at usegalaxy.org. We ran FastQC to ensure high Phred quality scores and low percentages (<5%) adapter content (Galaxy). Illumina Universal Adapter sequences were trimmed from raw sequencing reads using Cutadapt (Galaxy). Data were aligned to the hg38 genome model using RNA STAR and raw reads per gene were determined using featureCounts. Differential expression analysis was performed in R using DESeq2⁴⁴. Heatmaps containing TPM values were generated using the pheatmap package in R. Comparison of the transcriptomic profile of N1ICD overexpressing EPCs±Dox subpopulations to transcriptomes of established cell and tissue types was performed with PACNet⁴⁵.

Analysis of Published scRNA-Seq Data

Three publicly available single cell RNA-sequencing (scRNA-seq) data sets^{25,46,47} were analyzed using the Seurat⁴⁸ package in RStudio. We isolated endothelial cells from an integrative analysis of multiple in vivo human vascular single cell RNA-seq datasets²⁵, and performed differential expression analysis between brain endothelial cells and peripheral organ (i.e., heart, liver, lung, and skeletal muscle) endothelial cells. We identified a list of statistically significantly differentially expressed genes (false discovery rate<0.05) which included transcription factors and other regulatory factors as well as blood-brain barrier marker genes.

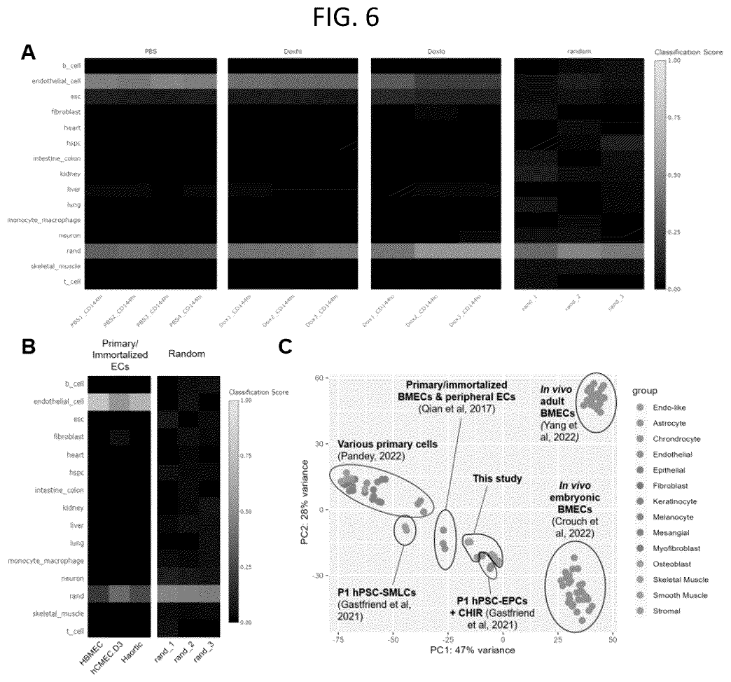

To compare gene-edited PB-TRE-N1ICD hPSC-EPCs treated with CHIR with or without doxycycline to in vivo brain microvascular endothelial cells (FIG. 6C), we obtained embryonic⁴⁶ and adult⁴⁷ brain vascular scRNA-seq datasets. Within each data set, we subsetted Seurat objects corresponding to capillary endothelial cells based on metadata provided by the authors. In order to make these single cell datasets comparable to the bulk RNA-seq in vitro datasets, we pseudo-bulked the single cell transcriptomes for each gestational time point except GW20 (very few capillary ECs) in the Crouch et al⁴⁶ dataset and all healthy control adult samples from Yang et al data set⁴⁷.

### Statistics

Statistics for most experiments were performed using Microsoft Excel or GraphPad Prism software. Student's t-test was used to compare differences of means between 2 groups. One-way ANOVA with post-hoc Tukey's or Dunnet's test was used to compare differences of means between 3 or more groups. For bulk RNA-sequencing analysis, p-values were calculated using the DESeq2 Wald test with Benjamini-Hochberg correction.

### Results:

Characterization of Transcytosis-Associated Markers in hPSC-EPCs with Lentiviral Overexpression of N1ICD

hPSC-derived EPCs stimulated with CHIR have previously been shown to gain substantial BBB character⁸ and are known as hPSC-derived CNS-like ECs. However, these CNS-like ECs do not accurately recapitulate the transcytosis properties of in vivo BMECs. Notch signaling, particularly that mediated by D114 ligand interaction with the Notch1 receptor in endothelial cells, has been implicated in induction and maintenance of low transcytosis in retinal endothelial cells in adult mice⁴; retinal endothelial cells contain blood-retina barrier properties, similar to BBB properties in BMECs. We initiated our investigation of the effect of N1ICD overexpression in unedited hPSC-derived EPCs (FIG. 11A) cultured in the presence of CHIR that were transduced with N1ICD or GFP (empty control) LV (FIG. 11B). Lentivirus doses and timing of transduction were titrated for both N1ICD LV and GFP LV to achieve a transduction efficiency of at least 70-80% and minimal cellular toxicity with smallest possible dose. We determined that hPSC-EPCs that were transduced with 6.25 μL/well GFP LV in a 6-well plate achieved a transduction efficiency (% GFP+ cells) of nearly 100% with essentially no reduction in CD31 MFI (FIG. 11D). We attempted transduction of hPSC-EPCs with N1ICD LV on both D5 and D7. Transduction efficiency was high on D11 both 4 and 6 days following transduction. However, only after 4 days of transduction were the resulting endothelial cells still 100% CD31+ (data not shown). When hPSC-EPCs were transduced with N1ICD LV on D7, we observed ˜90% transduction efficiency and ˜100% CD31+ cells, although events corresponding to cells with higher GFP MFI also had a slight reduction in CD31 MFI (FIG. 11C).

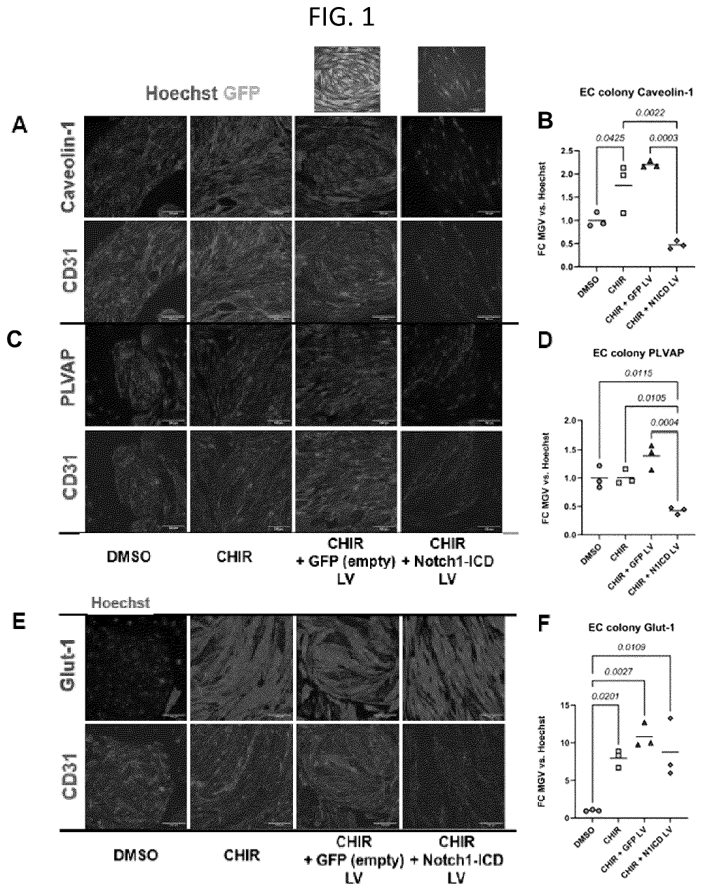

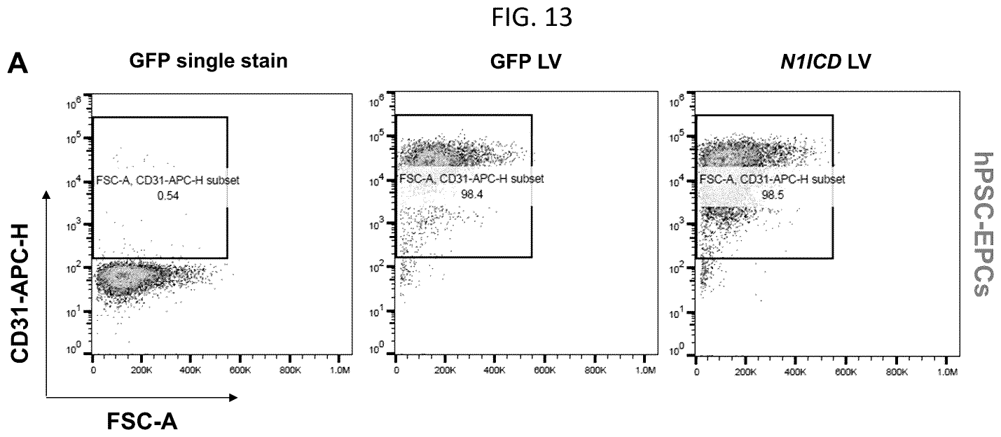

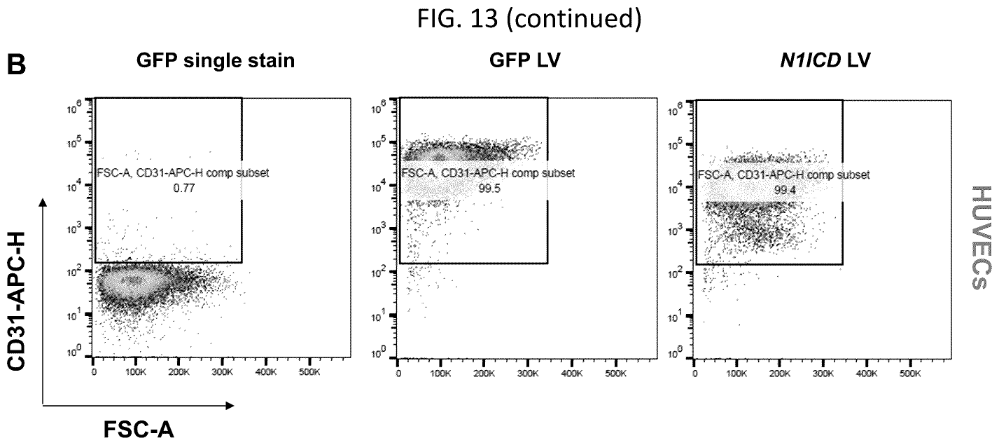

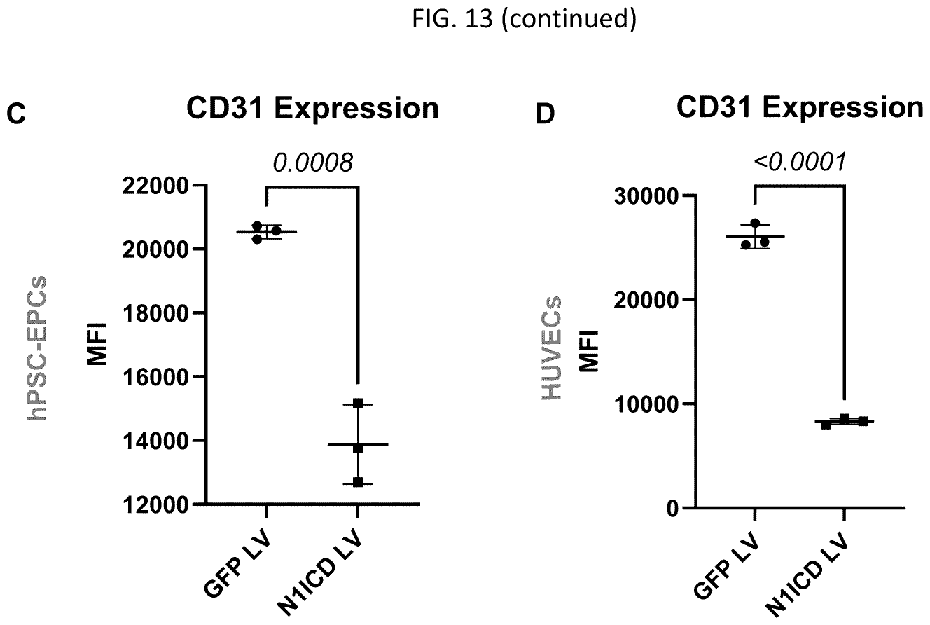

After identifying the appropriate dosage and timing for transduction of hPSC-EPCs with N1ICD LV and GFP LV, we measured expression of key vesicular transcytosis-associated structural proteins caveolin-1⁴⁹ and PLVAP⁵⁰ by immunostaining. We expect reduced expression of these proteins in brain microvascular endothelial cell (BMEC)-like cells compared to peripheral-like ECs⁵¹. hPSC-EPCs that were treated with both 4 μM CHIR and transduced with N1ICD LV had significantly lower expression of caveolin-1 than those treated with CHIR alone or treated with CHIR and GFP LV (FIG. 1A,B). There was also a significant reduction in PLVAP relative to these two controls and compared to the vehicle control (FIG. 1C,D). We performed immunoblotting for Notch1 protein in CHIR+GFP LV and CHIR+N1ICD LV-treated hPSC-EPCs (FIG. 11E). While there was no significant difference in full length Notch1 band intensity between conditions, we observed a non-significant increase in Notch1 ICD in cells that had been transduced with N1ICD LV (FIG. 11F,G). We also confirmed by flow cytometry that hPSC-EPCs treated with CHIR and N1ICD LV have significantly reduced caveolin-1 expression as compared to CHIR and GFP LV-treated hPSC-EPCs (FIG. 12A,C). Additionally, given that CHIR leads to strong upregulation in BBB-enriched glucose-transporter GLUT-1 relative to the vehicle in hPSC-EPCs⁸, we confirmed that the significant upregulation in GLUT-1 expression was maintained in hPSC-EPCs treated with CHIR and transduced with N1ICD LV relative to vehicle (FIG. 1E,F). Because a previous study demonstrated that earlier passage hPSC-EPCs were more responsive to CHIR-mediated GLUT-1 upregulation⁸, we tested whether hPSC-EPCs would be more responsive to N1ICD LV-mediated reduction in caveolin-1 due to their relative immaturity compared to terminally differentiated peripheral-like ECs. However, we also observed significant reduction in caveolin-1 expression in HUVECs treated with CHIR and N1ICD LV (FIG. 12B,D), demonstrating that this combination could also reduce transcytosis in mature ECs lacking any intrinsic BBB properties. Furthermore, although hPSC-EPCs treated with CHIR and N1ICD LV retained expression of CD31, we observed reduced CD31 fluorescence intensity in immunostained cells (FIG. 1) and confirmed this finding in both hPSC-EPCs and HUVECs transduced with N1ICD LV by flow cytometry (FIG. 13). Thus, N1ICD can decrease transcytosis related proteins in concert with Wnt signaling activation in hPSC-derived CNS-like ECs.

We then measured expression of several BBB-related genes in hPSC-EPCs treated with CHIR and N1ICD LV by RT-qPCR. We observed a significant reduction in CAV1 and PLVAP expression (˜2-fold) and significant upregulation of MFSD2A expression (˜5-fold) (FIG. 2A-C). Although we observed a non-significant increase in N1ICD expression, this is likely because the primer used cannot distinguish between native and lentiviral N1ICD (FIG. 2D). GFP expression was also significantly lower in CHIR and N1ICD LV treated hPSC-EPCs compared to those treated with CHIR and GFP LV (FIG. 2E), mirroring our observations at the protein level (FIG. 1A). There was no difference in CDH5 expression, indicating that N1ICD LV transduced cells still retain their endothelial character (FIG. 2F). Because Notch signaling has the potential to drive endothelial to mesenchymal transition (EndMT)⁵², we assessed expression of EMT-related genes CDH2 and FN1; although there was no change in CDH2, there was a significant increase in FN1 in hPSC-EPCs treated with CHIR and N1ICD LV (FIG. 2G,H). However, FN1 is upregulated in BMECs relative to peripheral ECs^{25,53}, so increased expression may be a marker of improved BMEC character. There was no significant change in expression of tight junction-related genes CLDN5, OCLN, or TJP1 (FIG. 2I-K) or efflux-transporter genes ABCB1, ABCG2, or ABCC1 (FIG. 2L-N). A ˜2.5-fold significant increase was observed in SLC2A1 expression in hPSC-EPCs treated with CHIR+N1ICD LV compared to Wnt signaling activation alone (CHIR +GFP LV) (FIG. 2O). Overall, these data indicate that the primary effect of treatment of hPSC-EPCs with CHIR and N1ICD LV is a selective reduction in expression of vesicular transcytosis-related and nutrient transporter-related genes, whereas other key BBB axes related to physical barrier and efflux transporter properties remain unchanged.

Generation and Characterization of hPSC-Derived EPCs with Doxycycline-Inducible Overexpression of N1ICD

Because of the significant variability in N1ICD upregulation and reduction in CD31 expression observed in the hPSC-EPCs that had been transduced with NICD LV, we hypothesized that achieving more precise control over the gene dosage and duration of N1ICD overexpression would allow us to achieve significantly reduced expression of vesicular-transcytosis-associated markers (e.g., CAV1, PLVAP) while preserving the expression of endothelial markers CD31 and VE-cadherin (encoded by CDH5) that were observed in hPSC-derived CNS-like ECs generated by CHIR treatment. We sought to generate a hPSC cell line with doxycycline-inducible overexpression of N1ICD so that we could achieve specific, highly reproducible levels of overexpression by optimizing the doxycycline dose and timing in an hPSC line with a specific copy number of N1ICD overexpressing constructs. This hPSC line could then be differentiated to EPCs and sorted in a similar fashion as EPCs derived from unedited hPSCs to yield hPSC-derived CNS-like ECs with inducible N1ICD overexpression.

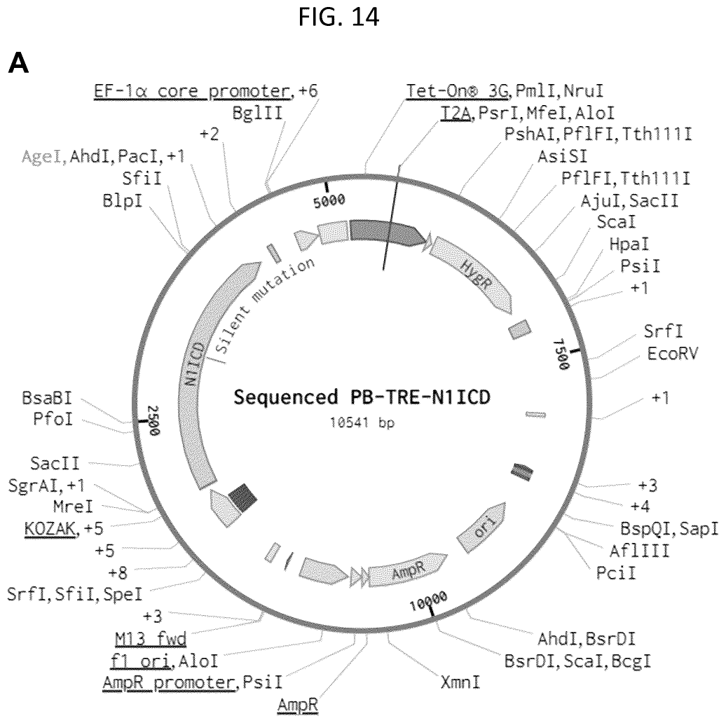

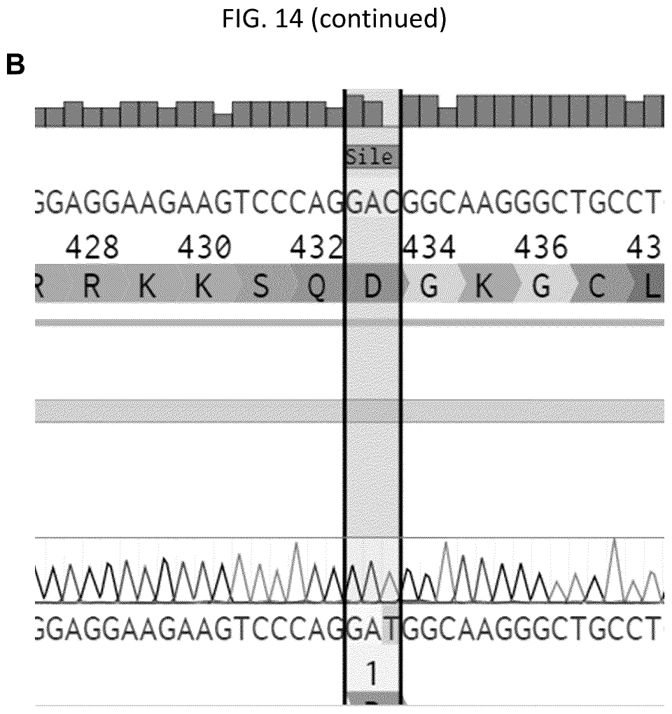

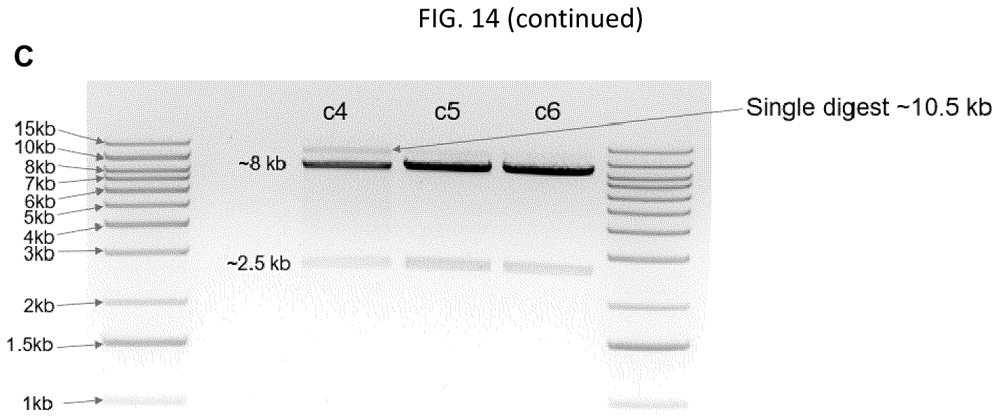

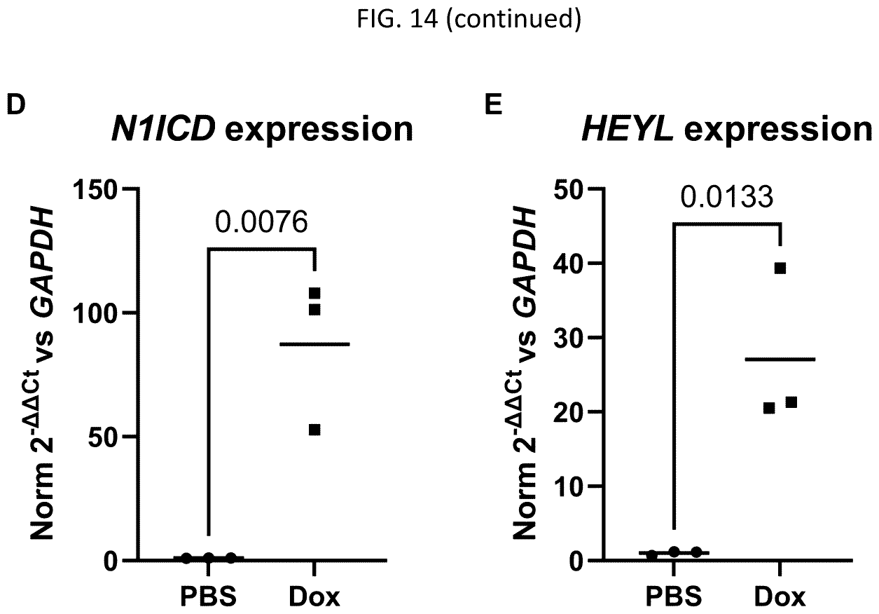

To generate an hPSC line with doxycycline-inducible N1ICD overexpression, we generated a piggyBac transposon vector that encoded a doxycycline-inducible N1ICD overexpression cassette (FIG. 14A). After cloning the PB-TRE-N1ICD plasmid with the same N1ICD insert in pWPI-N1ICD into a piggyBac transposon backbone containing a doxycycline-inducible TRE3G promoter, we verified by Sanger sequencing that the amino acids in the translated version of the N1ICD coding sequence matched that in pWPI-N1ICD (human native N1ICD) (FIG. 14B). We also confirmed by gel electrophoresis that restriction digest of PB-TRE-N1ICD produced 2 fragments corresponding to the expected size of the backbone (˜8 kb) and N1ICD insert (˜2.5 kb), respectively (FIG. 14C). Next, the PB-TRE-N1ICD plasmid was co-transfected into hPSCs with a plasmid encoding piggyBac transposase. After hygromycin selection of hPSCs with successful integration of the transposon encoded on PB-TRE-N1ICD, we demonstrated that treatment of the heterogeneous pool of gene-edited hPSCs with doxycycline resulted in a significant increase in expression of N1ICD as well as HEYL, a gene regulated downstream of Notch signaling (FIG. 14D,E). We attempted to differentiate the heterogeneous population of gene-edited doxycycline-inducible N1ICD overexpressing hPSCs to EPCs by a similar protocol to that used for differentiation of unedited hPSCs to EPCs (FIG. 3A). We were able to differentiate a population of gene-edited hPSC-EPCs with a similar purity to that observed in a similar differentiation of unedited hPSC-EPCs (˜15%). Furthermore, after positive selection of CD31+ cells by MACS, we obtained a ˜100% pure population of gene-edited hPSC-EPCs (FIG. 3C). We also demonstrated an increase in N1ICD expression in gene-edited hPSC-EPCs treated with CHIR and Dox by immunoblotting (FIG. 3D).

Next, we wanted to identify what the optimal timing was for treatment of gene-edited hPSC-EPCs with hygromycin and doxycycline. We hypothesized that because chromatin remodeling may have occurred over the course of the EPC differentiation, that regions of individual edited cells' genomes may have become less accessible in regions where the PB-TRE-N1ICD transposon had integrated. Thus, to maximize the overall population response to doxycycline-induced N1ICD overexpression, we would perform a hygromycin reselection on gene-edited hPSC-EPCs prior to doxycycline treatment. We cultured heterogeneous gene-edited hPSC-EPCs with 4 or 6 days of hygromycin reselection followed by 4 or 6 days of doxycycline. After the culture period was complete for each condition, cells were analyzed with CD31 flow cytometry. The condition with highest percentage of CD31+ cells and highest CD31 MFI (˜90%) was 6 days of hygromycin reselection followed by 4 days of doxycycline treatment (FIG. 15A,B). We then proceeded to culture heterogeneous gene-edited hPSC-EPCs in hECSR supplemented with CHIR and the optimized hygromycin reselection and doxycycline treatment timing and performed immunocytochemistry to measure expression of caveolin-1 and CD31 in doxycycline vs. PBS treated cells. Although there appeared to a be visual reduction in caveolin-1 fluorescence intensity, the variability in fluorescence intensity meant that there was not a significant reduction in caveolin-1 expression in the doxycycline-treated heterogeneous population of gene-edited hPSC-EPCs at the selected doxycycline dosage (FIG. 15C,D). However, there was also no difference in CD31 expression between conditions, indicating that the doxycycline-treated cells had maintained their endothelial character (FIG. 15C,D).

We next selected individual clones of gene-edited PB-TRE-NICD hPSC with specific copy numbers of the doxycycline-inducible N1ICD overexpressing transposon. We selected a total of 11 clones with piggyBac transposon copy numbers ranging from ˜2 to ˜48 copies. Most of the clones have copy numbers in the range of 10-25 copies (FIG. 16A). Four of these clones with a range of copy numbers (˜2 [9(+Y)], ˜15 [11 (+CR2)], ˜24 [10 (+CR2)], and ˜48 [3(+Y)] copies) were selected for further characterization. We treated these clones at the hPSC level with either PBS or different concentrations of doxycycline ranging from 100 ng/ml to 1 μg/mL to determine if a dose-response existed in terms of N1ICD overexpression. We found that although there was a consistent increase in N1ICD expression for all 4 clones with the addition of increasing concentrations of doxycycline, the relative expression differences in N1ICD at similar doxycycline doses did not correlate with copy number of the PB-TRE-N1ICD transposon in each of the hPSC clones (FIG. 16B). This finding is likely attributable to variability in genomic location of piggyBac transposon integration and thus variability in accessibility of the transposon to transcriptional machinery and other local gene regulatory elements.

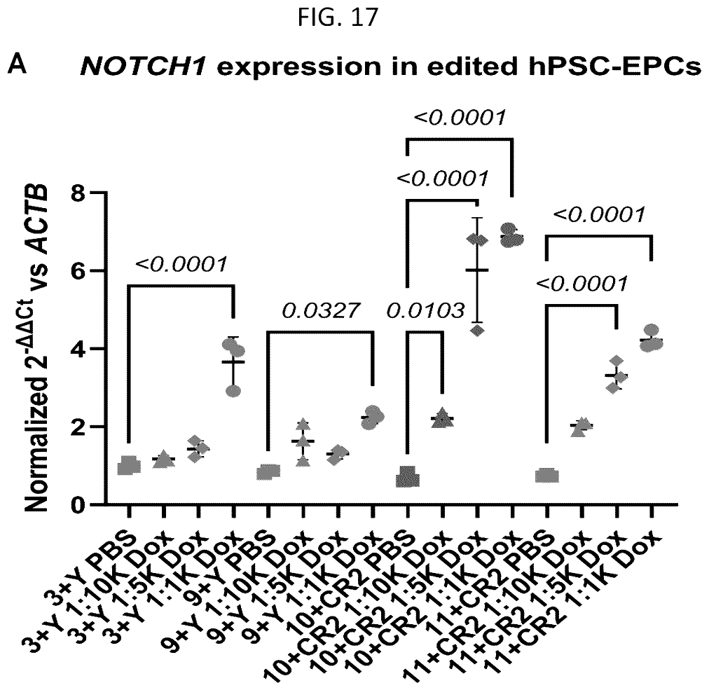

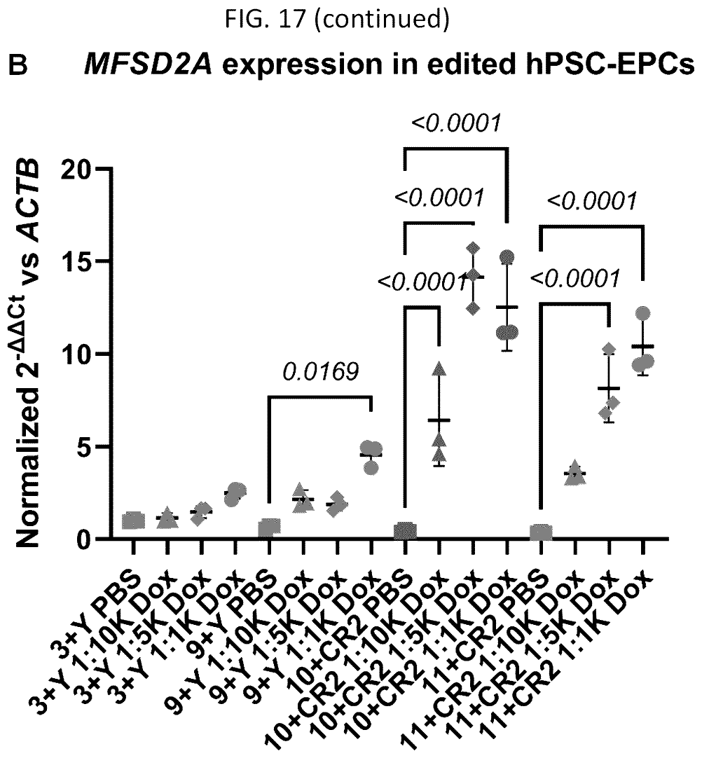

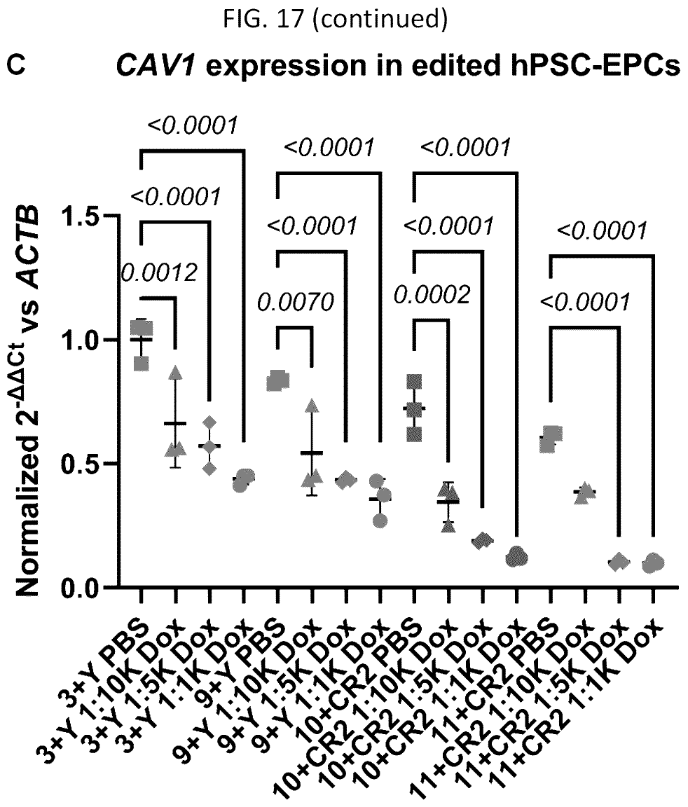

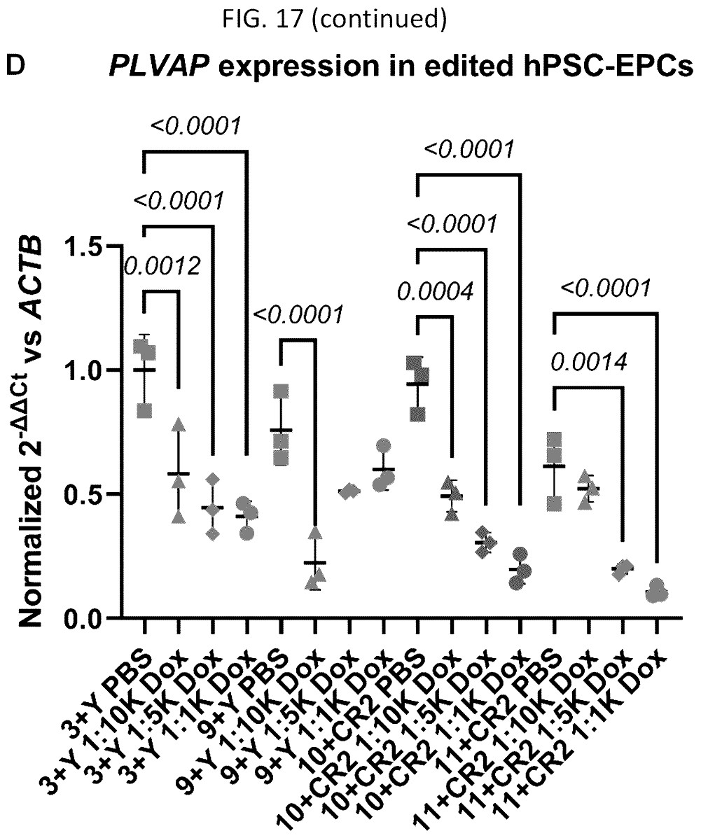

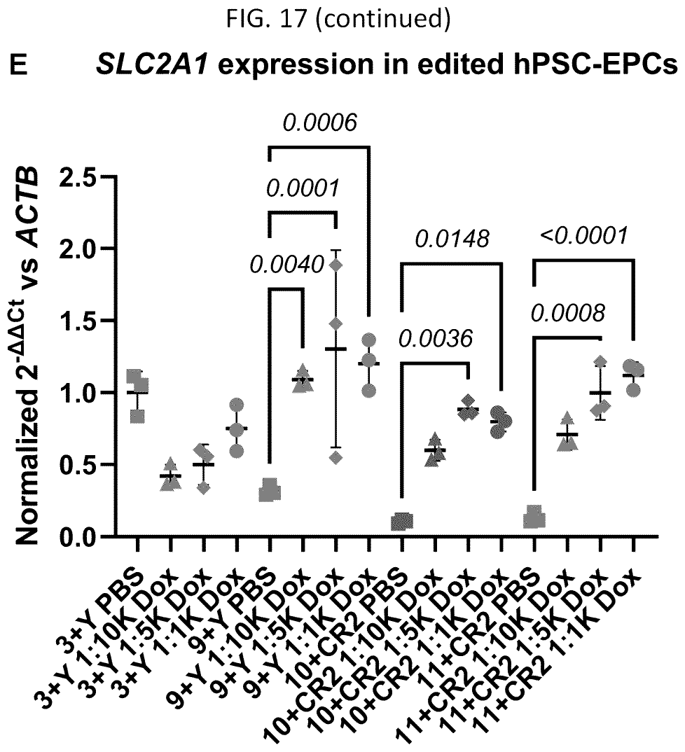

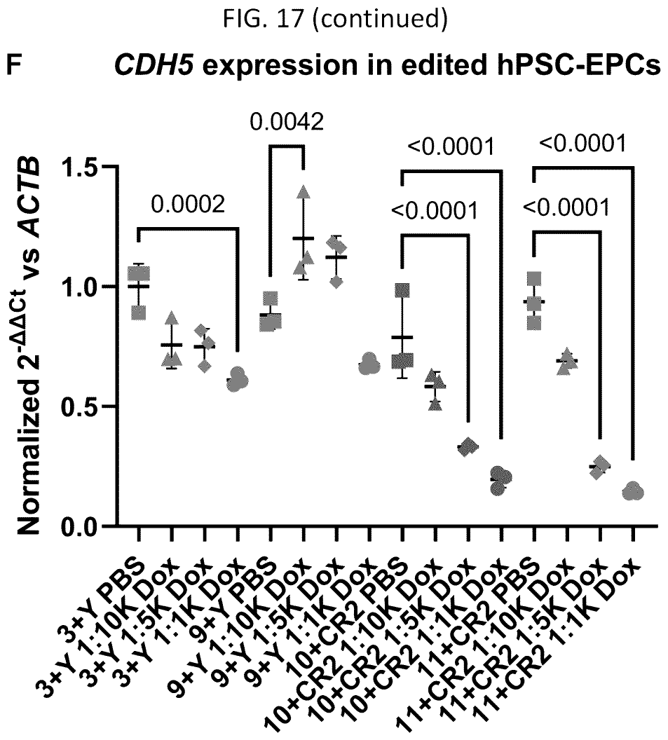

We also compared expression of selected BBB-related genes in hPSC-EPCs derived from gene-edited doxycycline-inducible N1ICD overexpressing clones 3(+Y), 9(+Y), 10(+CR2), and 11 (+CR2). The edited hPSC-EPCs were treated with 1:1000 diluted DPBS or doxycycline ranging from 100 ng/mL to 1 μg/mL from D11-D15 of culture (FIG. 3A). Significant, dose-dependent increases in NOTCH1 and MFSD2A expression were observed across all 4 clones (FIG. 17A,B). The highest degree of NOTCH1 and MFSD2A upregulation were observed in EPCs derived from clones 10(+CR2) and 11 (+CR2), which had intermediate copy numbers among the 4 clones analyzed. A dose dependent decrease in CAV1 expression (FIG. 17C) was seen in all 4 clonal edited hPSC-EPCs. Similarly, a decrease in PLVAP was seen with increasing doxycycline dose in all 4 lines except 9(+Y) hPSC-EPCs, which demonstrated peak reduction in PLVAP expression at the lowest doxycycline concentration (1:10K, 100 ng/ml), followed by an increase to just below the baseline (+PBS) expression level at the highest concentrations (1:5K ng/ml], 1:1K [1 μg/mL]) (FIG. 17D). We also observed a dose-dependent increase in SLC2A1 for all 4 lines except 3(+Y) (FIG. 17E). Among the 4 lines tested, the greatest reduction in CDH5 expression was observed in EPCs derived from 10(+CR2) and 11 (+CR2) edited hPSCs, correlating with the larger degree to which N1ICD is overexpressed in these two clones with increasing doxycycline dosage (FIG. 17F). There was ˜2 fold decrease in CDH5 expression at the highest doxycycline dose for the 3(+Y) hPSC-EPCs and a non-significant decrease for the 9(+Y) hPSC-EPCs. Taken together, these data suggest that upregulation of MFSD2A and SLC2A1 and downregulation of CAV1 and PLVAP are dependent on degree of Notch signaling activation via N1ICD overexpression. They also indicate that there is a trade-off between more BBB-like expression for genes related to vesicular transcytosis and selective nutrient transport and canonical endothelial marker expression (e.g., for CDH5, which encodes VE-cadherin).

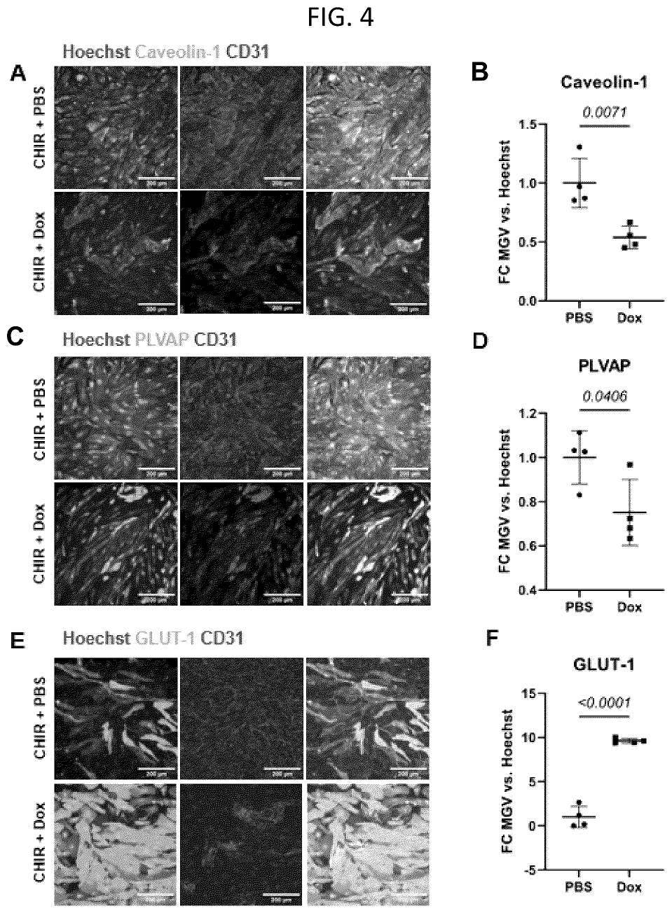

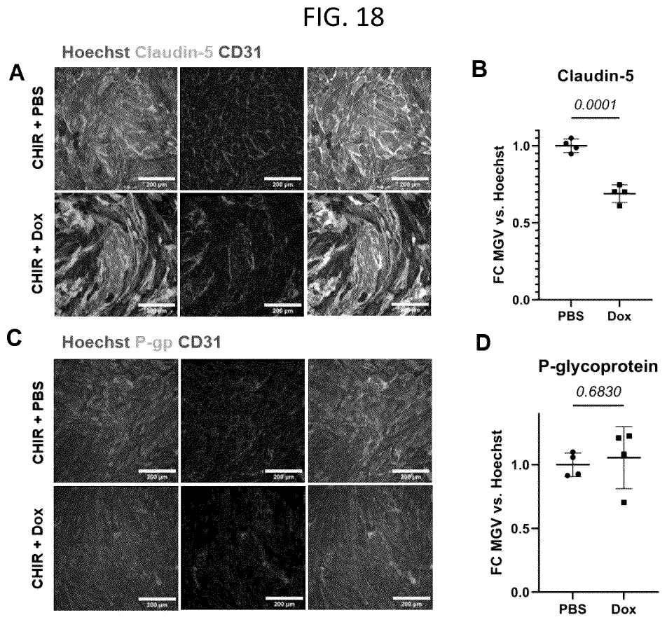

We continued with 1 μg/mL doxycycline or PBS control treatment of 10(+CR2) hPSC-EPCs for 4 days as this clone appeared to have the highest degree of N1ICD overexpression induction upon doxycycline addition. We confirmed a significant decrease in caveolin-1 (FIG. 4A,B) and PLVAP expression (FIG. 4C,D) and an increase in GLUT-1 expression (FIG. 4E,F) by immunocytochemistry in EPCs derived from 10[+CR2] treated doxycycline compared to the PBS control. We also observed patchy reduction in CD31 expression in the doxycycline-treated populations, with CD31 expression at similar levels to the PBS control in some colonies and significantly reduced to absent CD31 expression in cells outside of these colonies. We also observed reduced claudin-5 expression and no difference in P-glycoprotein (P-gp) expression in the Dox-treated cells relative to PBS control (FIG. 18A-D). Occludin expression was undetectable by immunocytochemistry both in the PBS control and Dox-treated cells (data not shown).

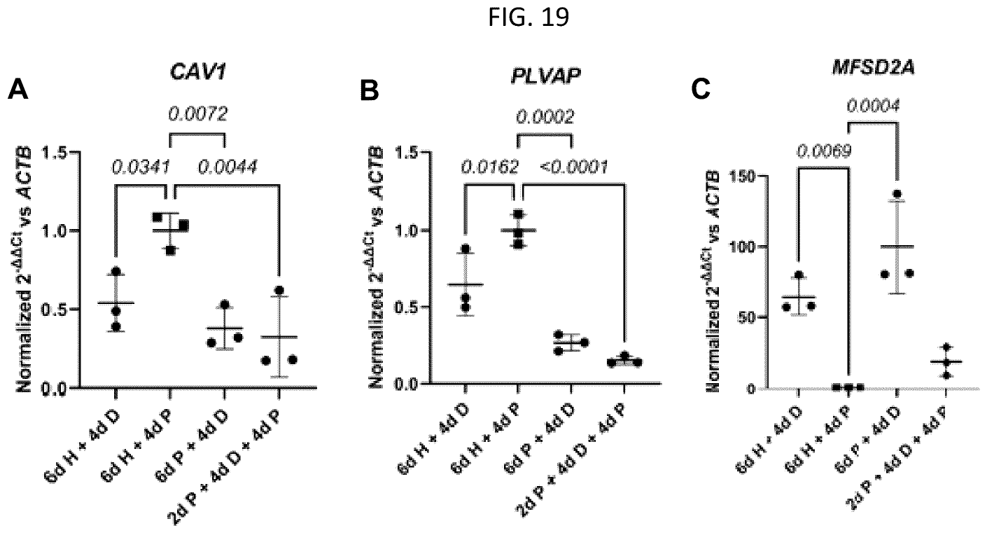

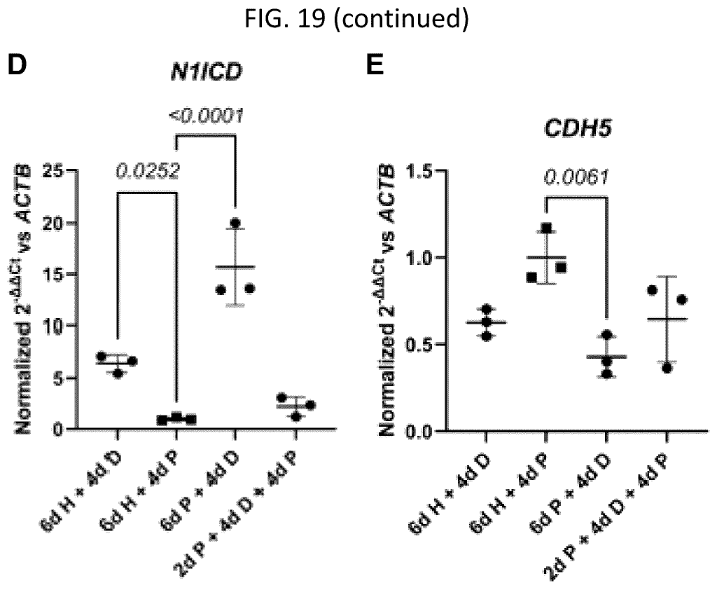

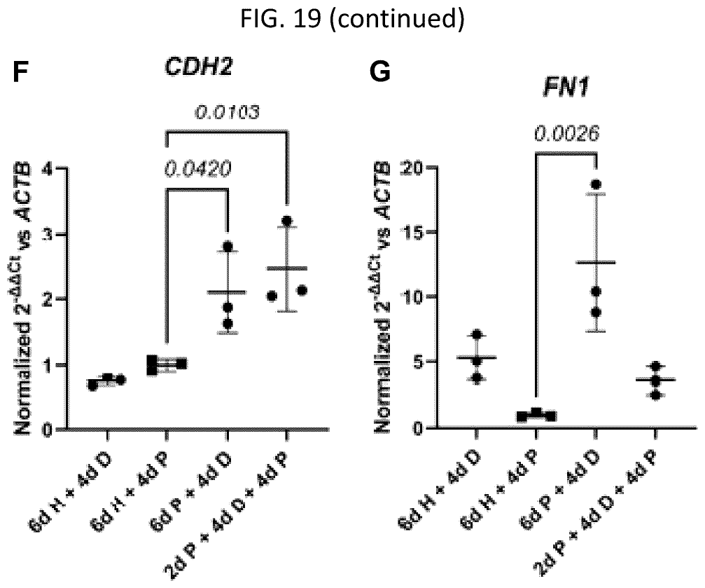

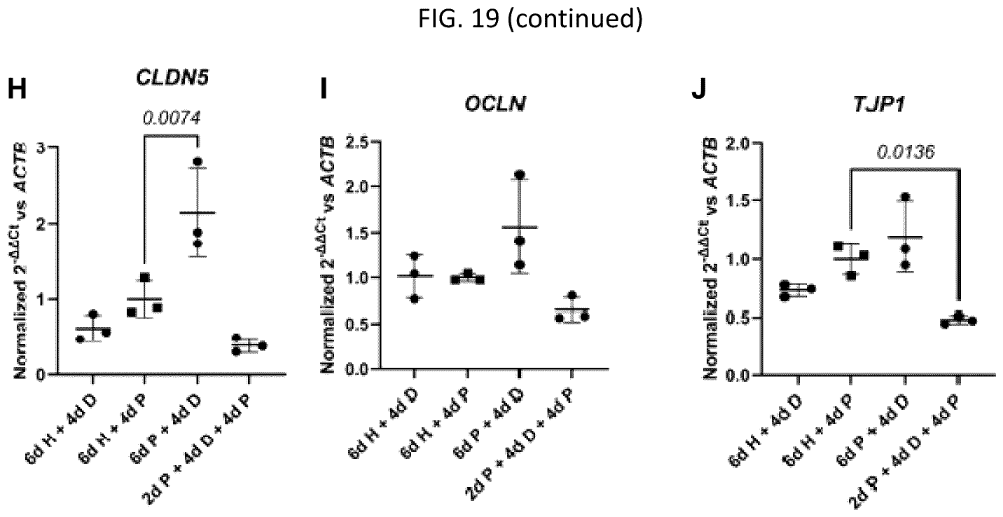

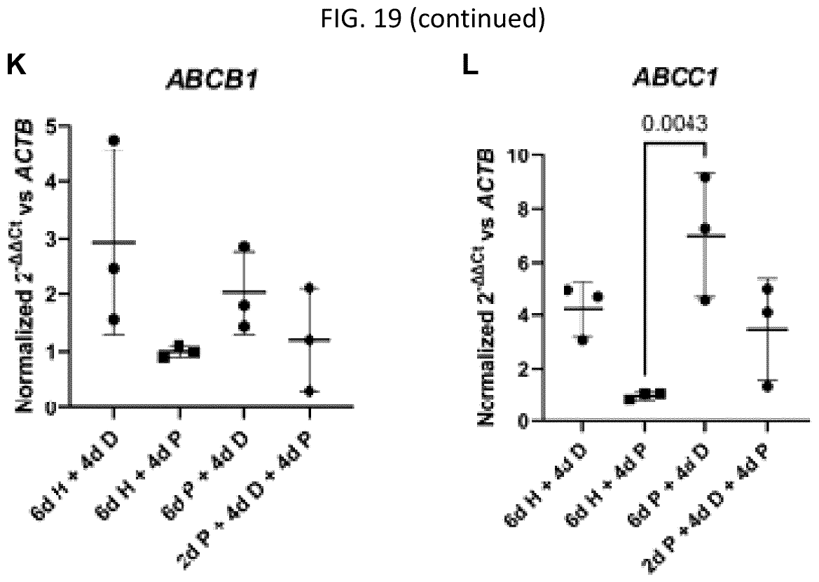

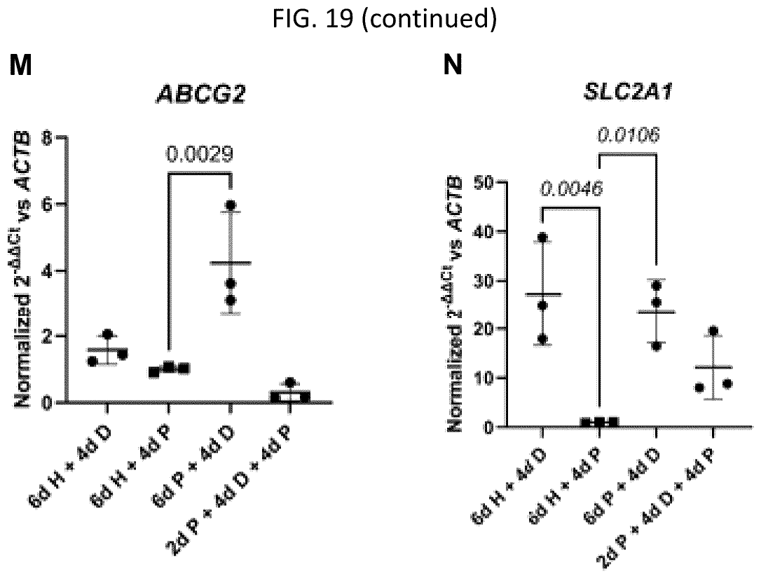

In PB-TRE-N1ICD hPSC-EPCs derived from the 10(+CR2) clone, we asked whether hygromycin reselection, or the lack thereof, and the timing of doxycycline induction of N1ICD overexpression in these edited EPCs would result in more BBB-like transcript-level changes. Because of the time frame for treatment identified in the heterogeneous population of edited PB-TRE-N1ICD hPSC-EPCs (FIG. 15), we tested 6 days of hygromycin reselection followed by 4 days of PBS (6d H+4d P) or doxycycline (6d H+4d D) treatment, as well as 6 days without hygromycin reselection followed by 4 days of doxycycline (6d P+4d D) treatment. Because using the edited doxycycline-inducible hPSC line allowed us to turn N1ICD overexpression on and off at will, we tested how induction of N1ICD overexpression 4 days earlier than the other conditions for the same duration (4 days) (2d P+4d D+4P) would affect BBB gene expression. While earlier induction of N1ICD overexpression (2d P+4d D+4P) resulted in similar significant reductions in CAV1 and PLVAP expression compared to the PBS control (6d H+4d P) as later N1ICD induction, both with and without hygromycin reselection (6d H+4d D and 6d P+4d D) (FIG. 19A,B), the earlier induction of N1ICD overexpression failed to yield a significant increase in MFSD2A (FIG. 19C) or NOTCH1 expression (FIG. 19D) that resulted when N1ICD overexpression was induced later and maintained without doxycycline withdrawal until the cells were isolated for analysis. However, we did observe an impressive 60- to 100-fold upregulation in MFSD2A in both conditions with later induction of N1ICD overexpression (6d H +4d D and 6d P+4d D) (FIG. 19C). A significant reduction in CDH5 was only observed in the 6d P+4d D condition (˜2-fold) relative to PBS control (FIG. 19E). However, similar although non-significant fold reductions in CDH5 were observed for other doxycycline-treated conditions (˜1.7-fold). A modest but significant increase (˜2-fold) in N-cadherin encoding CDH2 was observed in both the 6d P+4d D and 2d P+4d D+4P conditions (FIG. 19F), while FN1 was only significantly increased relative to PBS control (˜12-fold) in the 6d P+4d D condition (FIG. 19G). We next examined CLDN5, OCLN, and TJP1, which encode BBB-relevant tight junctions and tight junction-associated proteins. CLDN5 was non-significantly reduced in all conditions relative to the PBS control except in 6d P+4d D, in which CLDN5 was significantly upregulated (˜2-fold) (FIG. 19H). There was no significant difference in OCLN expression in any of the doxycycline-treated conditions relative to PBS control, although there was a slight upward trend with 6d P+4d D and slight downward trend with 2d P+4d D+4P (FIG. 19I). TJP1 was unchanged in most of the doxycycline-treated conditions except for 2d P+4d D+4P, in which TJP1 was significantly downregulated (˜2 fold) (FIG. 19J). There was no significant increase in expression of the efflux transporter genes ABCB1, ABCC1, and ABCG2 between the doxycycline treated conditions and PBS control, except for a 7-fold and 4-fold increase in ABCC1 and ABCG2, respectively in 6d P+4d D (FIG. 19K-M). Finally, there was a significant increase in SLC2A1 expression in both 6d P+4d D and 6d H+4d D relative to PBS control, but not in 2d P+4d D+4P (FIG. 19N). Taken together, these data suggest that hygromycin reselection is not required to achieve reduced expression of transcytosis structural genes CAV1 and PLVAP as well as increased expression of MFSD2A and SLC2A1 upon overexpression of N1ICD in the doxycycline-inducible system. Furthermore, earlier induction and withdrawal of N1ICD overexpression (in the 2d P+4d D+4d P condition) diminishes or abolishes the effects of this treatment on induction of BBB-like changes in the expression of transcytosis and nutrient transporter-related genes. Changes in endothelial and EMT-related gene expression in the doxycycline-inducible system are similar in trend to those observed with lentiviral N1ICD overexpression. These data further reinforce that there is no consistent effect of N1ICD overexpression on tight junction or efflux transporter gene expression.

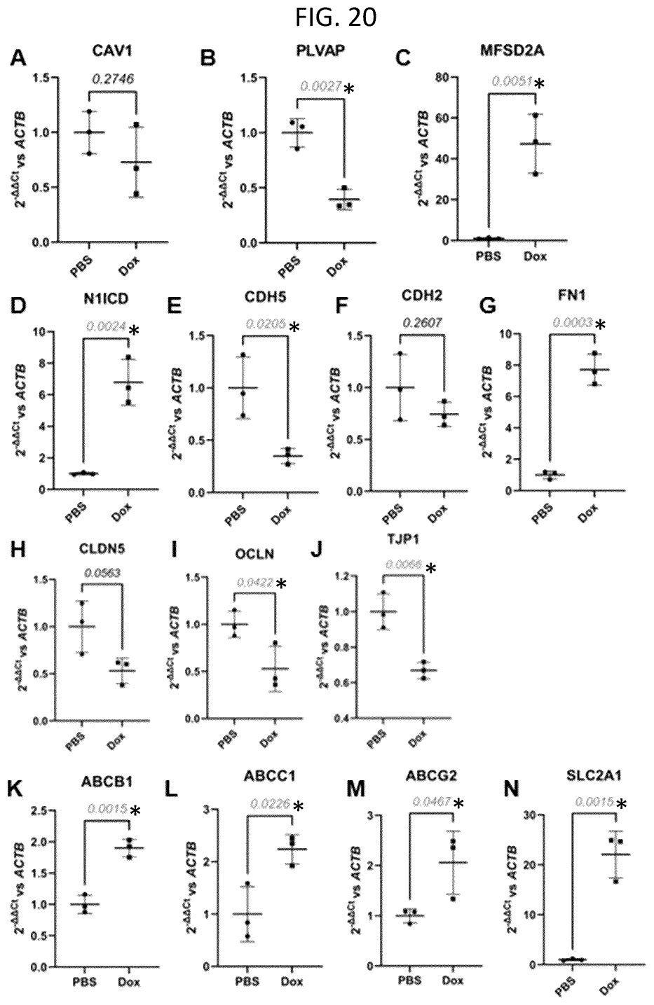

We selected the 10(+CR2) PB-TRE-N1ICD hPSC-EPCs treated with CHIR with or without doxycycline for 4 days (6d H+4d D) to follow up on via RT-qPCR. While we did not observe a significant reduction in CAV1 (FIG. 20A) due to increased variability between replicates, there was a significant reduction in PLVAP expression (FIG. 20B) and a significant, ˜50-fold increase in MFSD2A expression (FIG. 20C), compared to a ˜5-fold MFSD2A upregulation in N1ICD LV transduced cells. We observed significant upregulation in N1ICD expression (˜8-fold, FIG. 20D), with a fold change ˜2 times greater than with the lentiviral overexpression system (FIG. 2D). We also observed a significant, ˜3-fold decrease in expression of CDH5, which encodes VE-cadherin (FIG. 20E). Although there was no significant change in CDH2 expression (FIG. 20F), similar to the lentiviral approach, there was a significant increase in FN1 expression (FIG. 20G), although with a slightly lower fold increase than with the lentiviral system (FIG. 2H). Our RT-qPCR analysis showed no significant change in CLDN5 (FIG. 20H) due to doxycycline-induced N1ICD overexpression and significant downregulation of both OCLN (FIG. 20I) and TPJ1 (FIG. 20J). Finally, we noted significant ˜2-fold increases in expression of all genes encoding BBB-enriched efflux transporters, including ABCB1, ABCC1, and ABCG2 (FIG. 20K-M), which contrasted with the NICD LV transduced cells. There was a >20-fold increase in SLC2A1 expression compared to PBS-treated cells (FIG. 20N); both conditions are cultured in the presence of CHIR, which induces a ˜750-fold increase in normalized SLC2A1 expression (TPM) when supplemented in hPSC-EPCs culture medium⁸. These data generated with the 10(+CR2) hPSC line recapitulate that the major effect of overexpression of N1ICD in hPSC-derived CNS-like EPCs is the induction of more BBB-like expression patterns of transcytosis-related genes.

Sequencing Analysis of hPSC-Derived EPCs with Doxycycline-Inducible Overexpression of N1ICD.

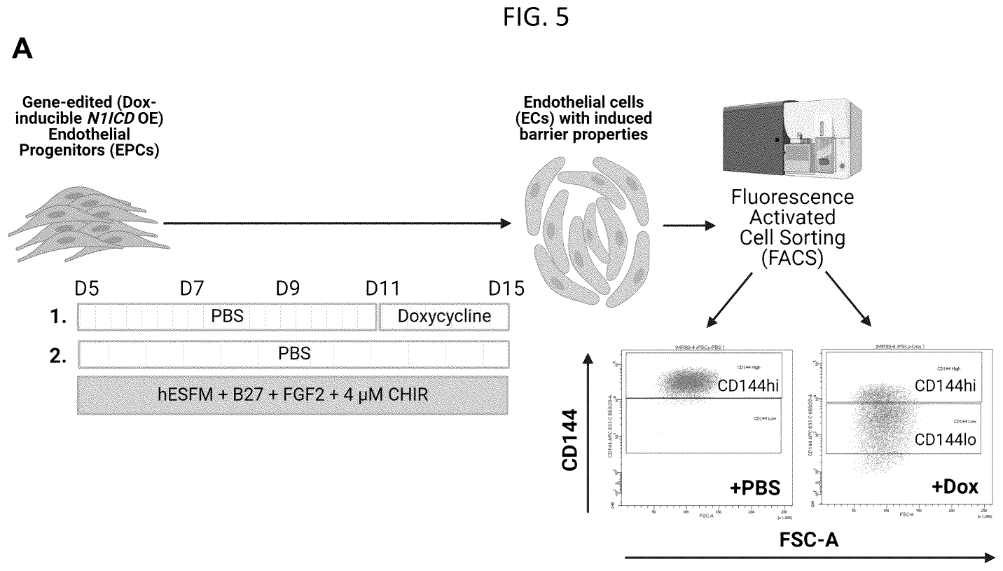

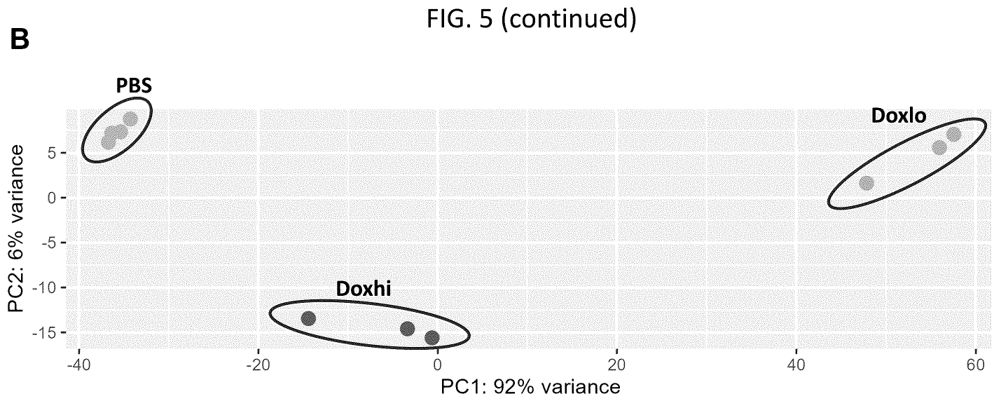

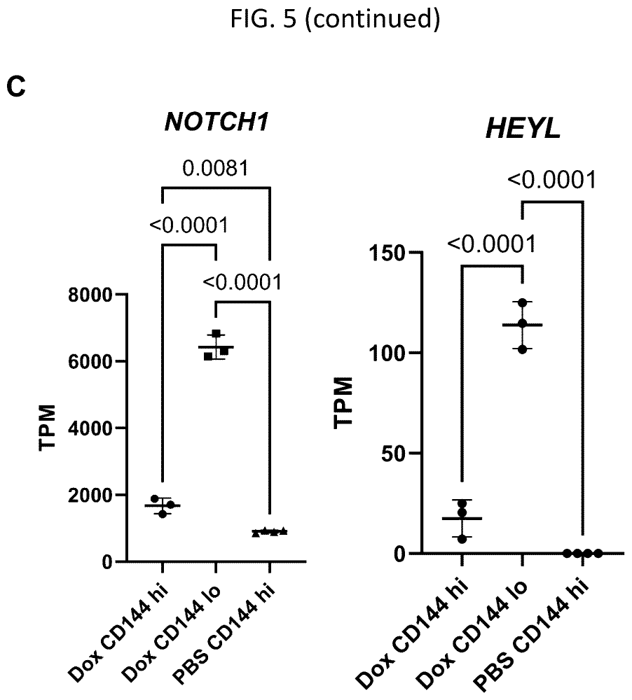

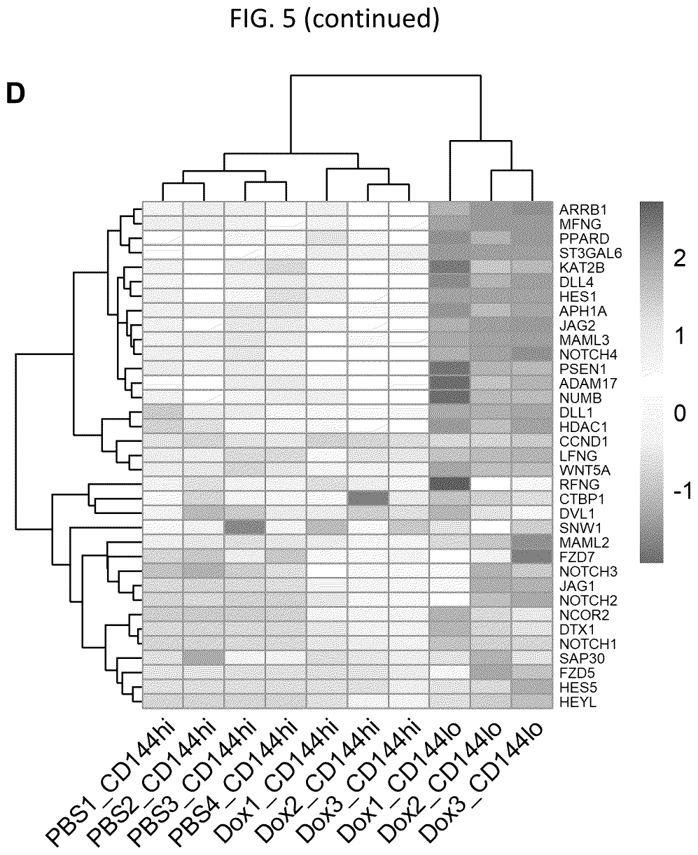

Next, we wanted to determine whether two subpopulations of endothelial cells existed in the gene-edited PB-TRE-N1ICD 10(+CR2) hPSC-EPCs treated with or without doxycycline, based on our observation of CD31 expression in immuno-stained cells derived from this line (FIG. 4). We performed fluorescence activated cell sorting (FACS) on PBS- or doxycycline-treated 10(+CR2) edited hPSC-EPCs based on surface expression of CD144 (also known as VE-cadherin), another canonical endothelial surface marker protein (FIG. 5A). The majority (˜94%) of the live cells in the PBS-treated condition were CD144+. In the doxycycline-treated population, a majority (˜90%) of the cells were CD144+, but these cells appeared as two subpopulations, one with high CD144 expression (˜25%, similar to the vast majority of the PBS-treated cells) and one with low but not absent CD144 expression (˜65%) (FIG. 5A). After FACS, RNA was isolated from the CD144 high and low subpopulations for both the PBS and doxycycline-treated samples. The RNA concentration from the PBS-treated CD144 low subpopulation was undetectable, so RNA was isolated from only the PBS-treated CD144 high, doxycycline-treated CD144 high, and doxycycline-treated CD144 low subpopulations. Samples were sent to Novogene for paired-end read bulk RNA-sequencing.

Following pre-processing of raw sequencing data, samples from each of the three sequenced subpopulations were analyzed using DESeq2 to investigate the differences between each of the doxycycline-treated subpopulations (Dox-treated CD144 high and CD144 low) and the PBS control (PBS-treated CD144 high). When visualized using principal component analysis (FIG. 5B), the PC1 axis, which accounts for 92% of variance between samples, is the primary axis along which samples from the different subpopulations segregate. PBS-treated CD144 high (PBS), is most different from Dox-treated CD144 low (Doxlo) and slightly less different from Dox-treated CD144 high (Doxhi). This suggests that the PC1 axis defines the axis of increasing N1ICD overexpression level. This is confirmed when we look at the normalized expression (TPM) of NOTCH1 for each of the subpopulations (FIG. 5C). There is a ˜2-fold increase in NOTCH1 expression from PBS-treated CD144 high (PBS CD144 hi) to Dox-treated CD144 high (Dox CD144 hi) and a nearly 7-fold increase from PBS CD144 hi to Dox-treated CD144 low (Dox CD144 lo). These differences match the relative distances observed between the global transcriptome dimensional reductions of each of these subpopulations on the PCA plot. Furthermore, we observed an increase in expression of HEYL, a gene downstream of Notch signaling activation, that is proportional to the increase in NOTCH1 expression (FIG. 5C). We examined a list of genes derived from a combination of the Hallmark and KEGG Notch signaling pathway gene sets to see which of the Notch-related genes were significantly differentially expressed between the three sequenced subpopulations (FIG. 5D). 35 genes were differentially expressed, encompassing Notch receptors and ligands as well as Notch-signaling associated transcription factors. Around half of these genes appeared to up- or down-regulated in response to increasing degrees of N1ICD overexpression. All genes encoding Notch receptors were upregulated with greater N1ICD overexpression dosage except for NOTCH4, which was downregulated in the highest N1ICD overexpressing subpopulation (Dox CD144 lo). In contrast, differentially expressed Notch ligands, such as DLL4, JAG2, and DLL1, were primary downregulated with increasing N1ICD overexpression. Only JAG1 was upregulated in Dox CD144 lo. HEY/HES family transcription factors were variably differentially expressed; HEYL and HES5 were significantly upregulated and HES1 significantly downregulated in the subpopulation with the greatest degree of N1ICD overexpression.

Given the reduction in CD144 expression that was observed in a significant proportion of the doxycycline-treated 10(+CR2) PB-TRE-N1ICD hPSC-EPCs, we asked if the cells with reduced CD144 expression maintained their endothelial identity. We used Platform-Agnostic CellNet (PACNet)⁴⁵ to compare the transcriptomes of each of the sequenced PBS- and doxycycline-treated subpopulations to transcriptomes of several standardized cell and tissue types used by the PACNet algorithm (FIG. 6A). For each of the subpopulations, the classification score was highest for endothelial cells, and nearly zero for all other cell types, including ESCs or hPSCs, fibroblasts, and skeletal muscle. However, there was a nonzero classification score for a randomly generated cell type “rand” derived from the combination of multiple transcriptomes of the training cell types. The “rand” cells had classification scores of zero for all the defined cell types. The classification score decreased for endothelial cells and increased for the “rand” cells in the doxycycline-treated subpopulations with increasing degrees of N1ICD overexpression relative to PBS control. Compared to the PBS- or doxycycline-treated PB-TRE-N1ICD hPSC-EPCs, the endothelial classification scores of primary or immortalized human brain endothelial cells (HBMEC and hCMEC.D3, respectively), or primary human aortic endothelial cells (Haortic), were higher and the corresponding “rand” cell classification scores were lower (FIG. 6B). Taken together these data suggest that while the transcriptome of the edited endothelial cells with or without N1ICD overexpression may not different from that of the generic endothelial cells used to train the PACNet algorithm, these cells are still endothelial cells and have not transformed into another cell type.

Additionally, we compared the global transcriptomes of the subpopulations that were sequenced as part of this study to the transcriptomes of other endothelial and non-endothelial cell types. The comparison bulk RNA sequencing datasets included Passage 1 hPSC-derived EPCs treated with CHIR8, Passage 1 hPSC-derived smooth muscle-like cells that were a biproduct of the hPSC-EPC differentiation⁸, primary/immortalized human brain microvascular endothelial cells and primary human aortic endothelial cells⁵⁴ previously used in the PACNet analysis, and a variety of primary cells including endothelial, epithelial, smooth muscle, skeletal muscle, and fibroblast cells (Pandey et al, GEO accession: GSE190615). Furthermore, we generated pseudo-bulk transcriptomes of human brain capillary, arterial, and venous endothelial cells from adult⁴⁷ and embryonic⁴⁶ single cell RNA-sequencing datasets. We included all these datasets in a DESeq2 analysis to investigate whether the endothelial-like cells generated by overexpression of N1ICD in Wnt-signaling activation hPSC-EPCs were more similar to bona-fide endothelial cells or other non-endothelial cell types (FIG. 6C). Most of the samples from the various studies segregated along PC1 (47% of variance) which roughly ranged from in vitro cells with the least endothelial character on the left to in vivo endothelial cells with BBB properties on the right. The transcriptomes of the cells sequenced as part of this study were most similar to the Passage 1 hPSC-EPCs+CHIR; this is unsurprising because the gene-edited PB-TRE-N1ICD hPSC-EPCs treated with CHIR with or without doxycycline were derived by the same differentiation protocol as the Gastfriend endothelial data set. Notably, the hPSC-derived smooth muscle-like cells from the Gastfriend data set clustered more closely with the non-endothelial primary cells from the Pandey data set than with hPSC-derived EPCs they were co-differentiated with. Additionally, the cells from this study clustered more closely with the primary and immortalized brain endothelial cells from the Qian dataset compared to the in vivo brain endothelial cells from the Crouch and Yang datasets. These data indicate that the gene-edited PB-TRE-N1ICD hPSC-EPCs treated with CHIR with or without doxycycline retain their endothelial character but are still more similar to in vitro brain-like endothelial cells which have lost a considerable degree of BBB character compared to in vivo embryonic or adult human brain capillary endothelial cells.

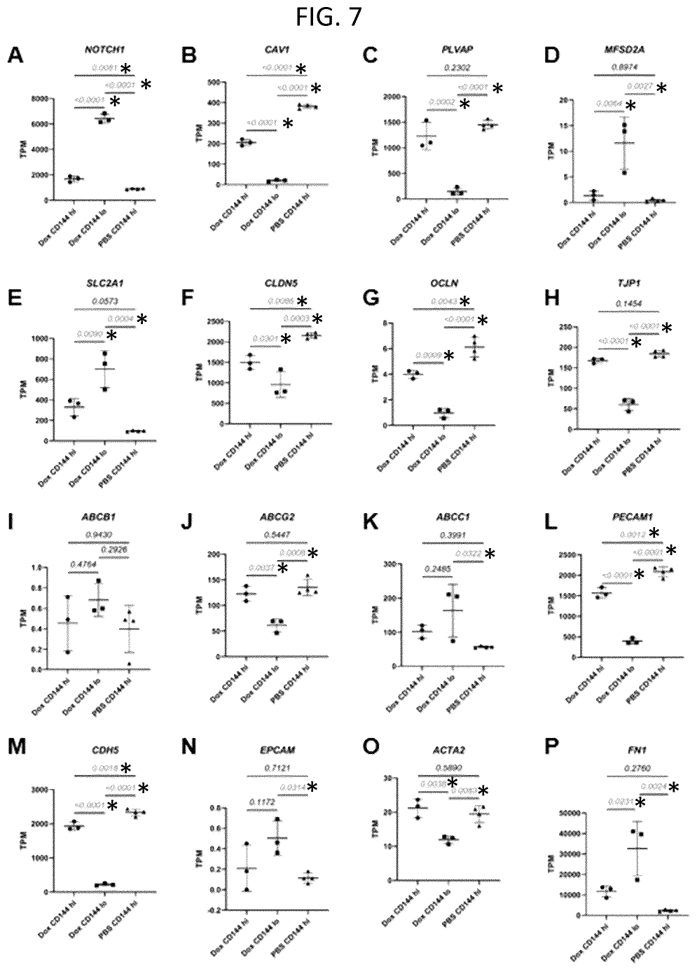

Next, we examined our sequencing data to determine the effects of doxycycline treatment, and N1ICD overexpression by extension, on BBB properties. Several of the differentially expressed genes from the qPCR analysis of gene-edited PB-TRE-N1ICD hPSC-EPCs treated with or without doxycycline (FIG. 19, FIG. 20) were also changed in the bulk RNA-seq analysis. In response to increasing N1ICD overexpression (ascending from PBS CD144 hi to Dox CD144 hi and Dox CD144 lo subpopulations), we observed decreasing CAV1 and PLVAP and increasing MFSD2A (FIG. 7B-D). SLC2A1 was also significantly increased upon N1ICD upregulation (FIG. 7E). However, we observed downregulation of tight junction genes CLDN5, OCLN, and TJP1 (FIG. 7F-H). Changes in efflux pump genes were more variable; ABCB1 was unchanged, ABCG2 decreased, and ABBC1 increased in response to N1ICD overexpression (FIG. 7I-K). Similar to previous assays, both in the doxycycline-inducible and lentiviral approaches for upregulating N1ICD, we noted significant decreases in endothelial genes PECAM1 and CDH5 (FIG. 7L-M). However, the normalized expression level for epithelial gene EPCAM was near 0 TPM, and while there was a significant increase in the Dox CD144 lo subpopulation with the greatest degrees of N1ICD overexpression, the highest normalized expression level observed was also less than 1 TPM (FIG. 7N), indicating a lack of epithelial identity in the resulting cells. We examined smooth muscle-associated transcript ACTA2 (FIG. 7O), and to our surprise observed a decrease in expression with increasing N1ICD overexpression. We observed an increase in FN1 (FIG. 7P), which although is a mesenchymal-associated gene, is enriched in BBB-like endothelial cells in vivo, based on comparison of scRNA-seq data of brain ECs to peripheral organ ECs²⁵.

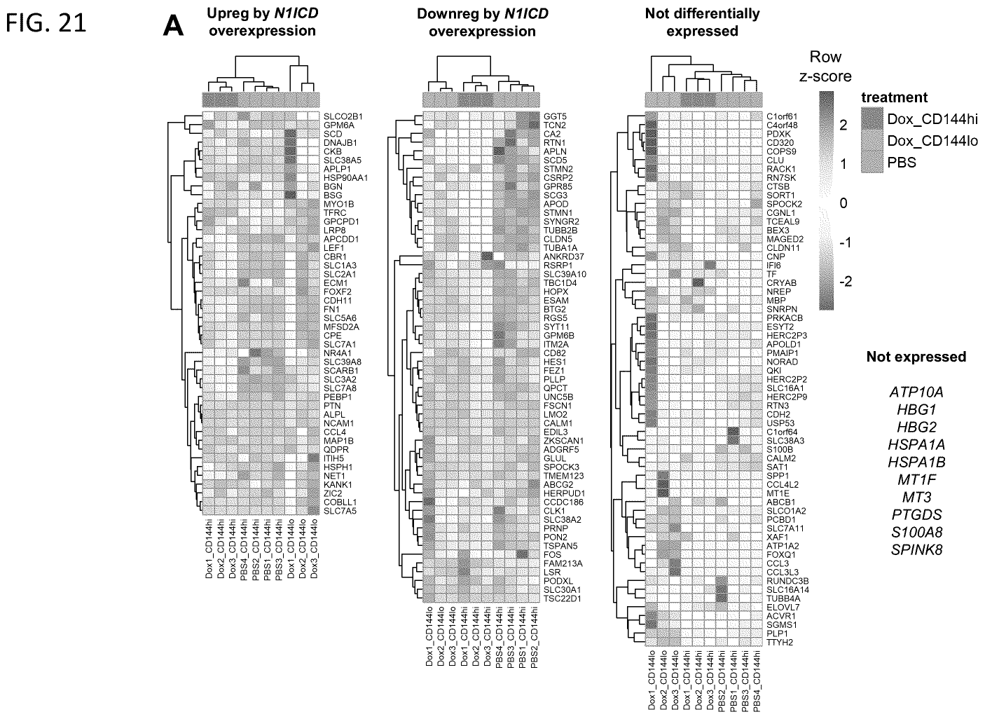

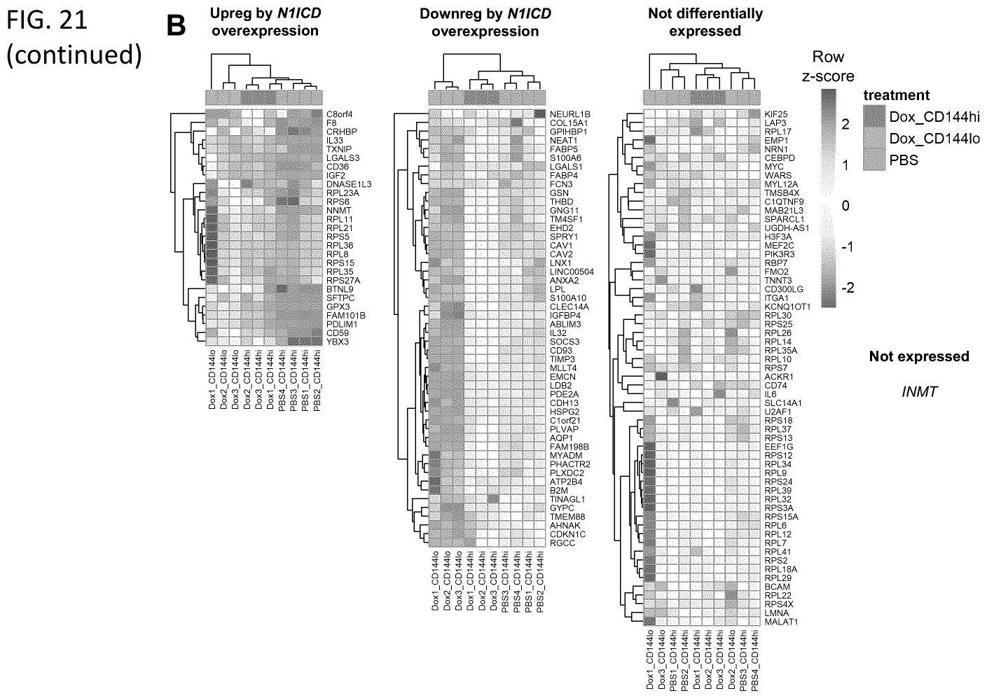

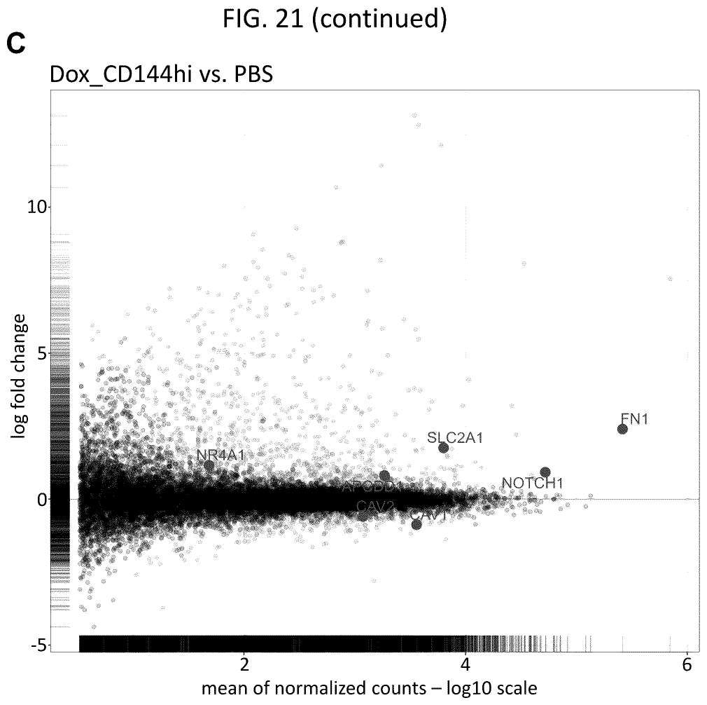

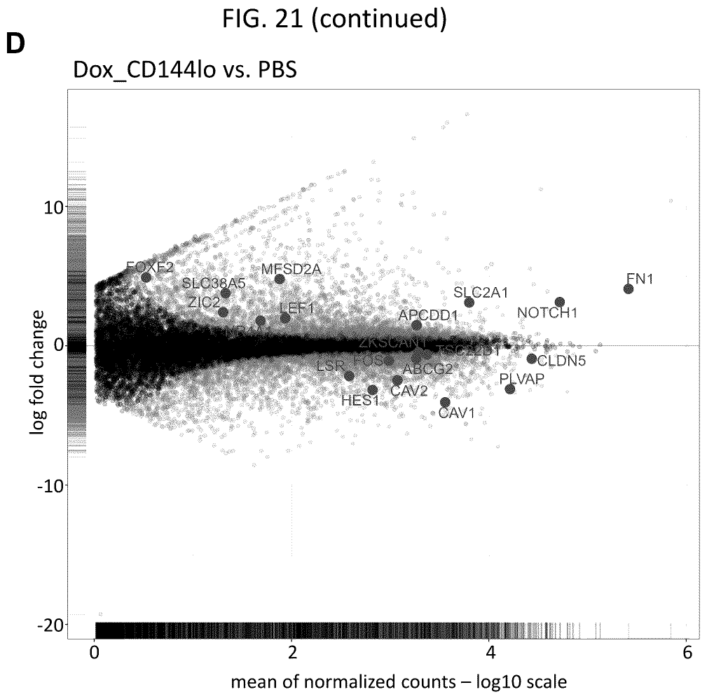

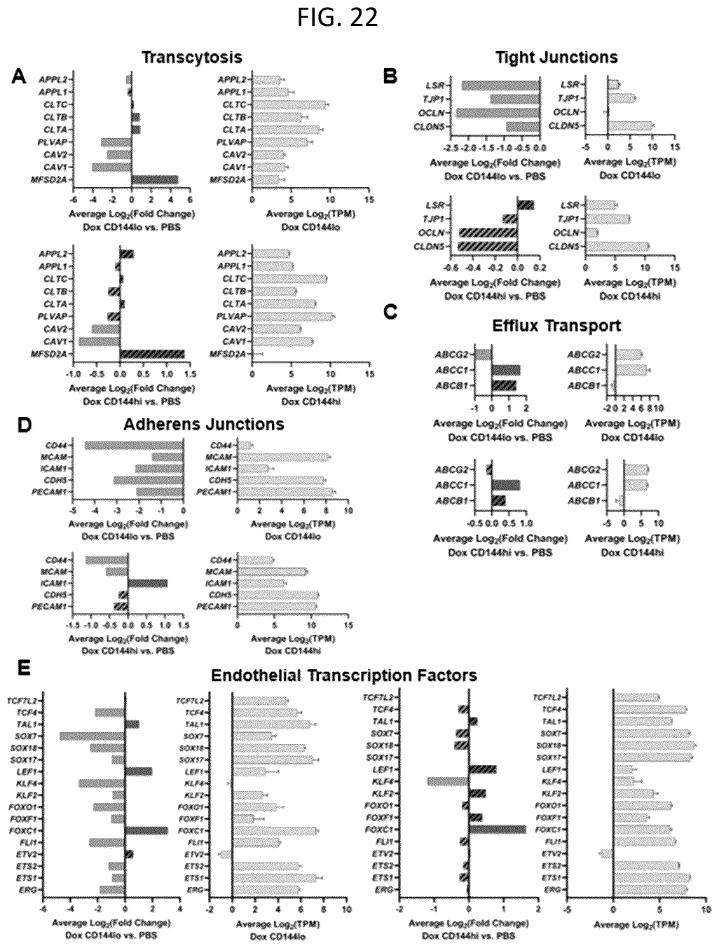

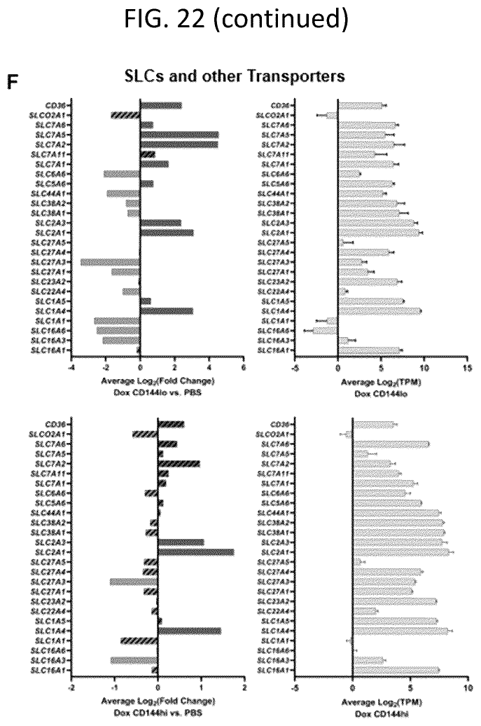

We next identified 310 genes which are predicted to up- or downregulated in brain endothelial cells relative to peripheral organ endothelial cells based on an integrated single-cell RNA-sequencing analysis of multiple publicly available datasets from several organs, including the brain²⁵. Of the 173 genes that were expected to be upregulated in brain vs. peripheral endothelial cells (FIG. 21A), 46 were appropriately increased in response to Notch signaling activation in our gene-edited PB-TRE-N1ICD hPSC-EPCs. These include SLC2A1 and MFSD2A, as previously shown, but also a host of other BBB-enriched genes, including many SLC nutrient transport genes such as SLC7A6, SLC7A5, SLC7A2, SLC7A1, SLC5A6, SLC2A3, SLC1A5, and SLC1A4 (FIG. 22F). 56/173 genes were inappropriately downregulated, including tight junction genes CLDN5, OCLN, TJP1, and LSR (FIG. 22B). 61/173 genes expected to be upregulated in BBB-like endothelial cells were not differentially expressed due to N1ICD overexpression. Of the 137 genes that were expected to be downregulated in brain vs. peripheral endothelial cells (FIG. 21B), 50 were appropriately downregulated including CAV1, CAV2 and PLVAP. When we examined transcytosis related genes more closely, caveolae-mediated transcytosis genes were primarily downregulated, whereas genes related to clathrin-mediated endocytosis and macropinocytosis (e.g., CLTA, CLTB, CLTC, and APPL1, respectively) were differentially expressed depending on the degree of N1ICD overexpression in the Dox-treated subpopulation (FIG. 22A). Generally, clathrin-related genes were more likely to be upregulated in response to increased Notch signaling. 27/137 were inappropriately upregulated and 59/137 were not differentially expressed due to N1ICD activation, although many of the genes in these two categories were not obviously associated with BBB phenotypes. Among both the genes that were expected to be up or downregulated at the BBB, several (11 total) were not expressed at all in our in vitro model (FIG. 21A-B). Several adherens junction genes besides CDH5 and PECAM1 were also downregulated in response to N1ICD overexpression, including CD44 and MCAM (FIG. 22D); however, the normalized expression of CDH5, PECAM1, and MCAM remained in the range of 200-500 TPM. We also noted decreased expression of several endothelial-associated transcription factors (FIG. 22E), although the normalized expression levels remained quite high despite the reductions due to N1ICD overexpression. The only transcription factors that were increased in response to Notch signaling activation were TAL1, LEF1, and FOXC1. In addition to genes corresponding to important BBB structural proteins and transporters, we also identified genes encoding transcription factors and other transcriptional regulatory factors (TFORFs) that were enriched in adult human brain endothelial cells compared to peripheral endothelial cells²⁵. We visualized differential expression of selected genes in Dox-treated gene-edited PB-TRE-N1ICD hPSC-EPCs vs. PBS control with an MA plot. These genes included TFORF and non-TFORF genes differentially expressed in brain endothelial cells in vivo (FIG. 21C-D). Unsurprisingly, more genes were significantly differentially expressed in the Dox CD144 lo subpopulation (which had higher N1ICD overexpression) vs. PBS control relative to the Dox CD144 hi vs. PBS comparison. In both comparisons, NOTCH1 was significantly upregulated, and several other brain-enriched TFORFs, including NR4A1, APCDD1, ZIC2, LEF1, and FOXF2 were upregulated due to N1ICD overexpression. Downregulated TFORF genes in the Dox-treated subpopulation with the highest degree of N1ICD overexpression included FOS, ZKSCAN1, TSCD22D1, and HES1 (FIG. 21C-D).

### Functional Characterization of Vesicles and Endocytic Trafficking.

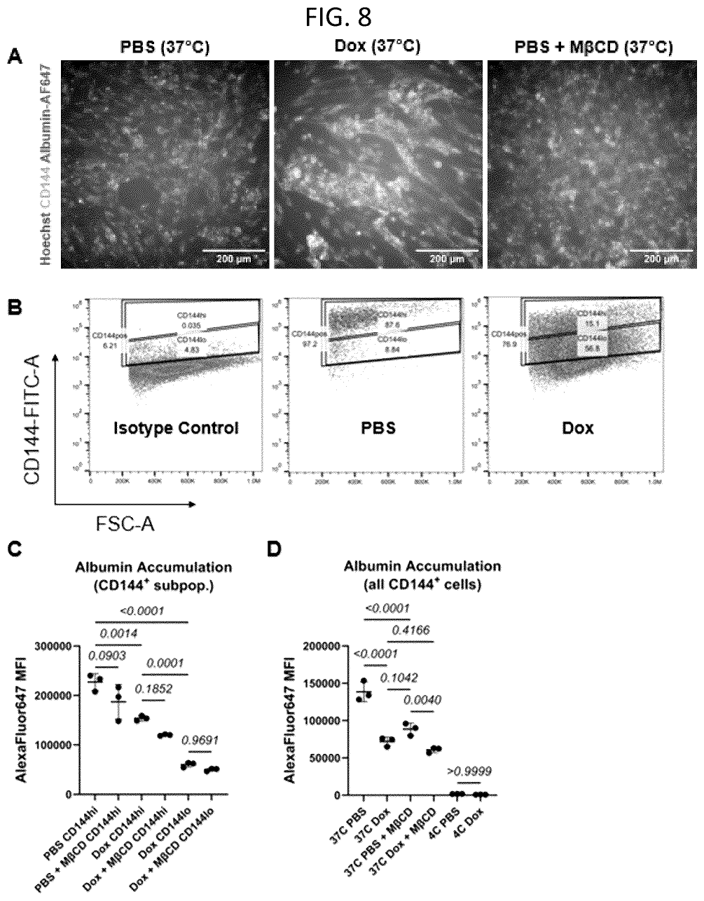

We next wanted to confirm that reduced caveolin-1 expression in both hPSC-derived endothelial progenitors and HUVECs with N1ICD overexpression correlated with decreased vesicle-based trafficking of substrates. We performed fluorescent substrate accumulation assays with albumin (˜66 kDa) conjugated to AlexaFluor 647 in gene-edited PB-TRE-N1ICD hPSC-EPCs treated with or without doxycycline. We imaged accumulation of fluorescent albumin by epifluorescence microscopy (FIG. 8A) and performed flow cytometry to quantify accumulation (FIG. 8B). Similarly to FACS-sorted Dox-treated gene-edited PB-TRE-N1ICD hPSC-EPCs (FIG. 5A), we observed that most PBS-treated cells had high CD144 expression, whereas Dox-treated cells had subpopulations with high or low CD144 expression (FIG. 8B). We quantified fluorescent albumin accumulation in the PBS CD144 hi, Dox CD144 hi, and Dox CD144 lo subpopulations, and found that there was a trend of decreasing albumin accumulation with increasing levels of N1ICD overexpression (FIG. 8C). When all CD144+ cells were considered, there was significant reduction in fluorescent albumin accumulation due to doxycycline treatment, and by extension Notch signaling activation (FIG. 8D).

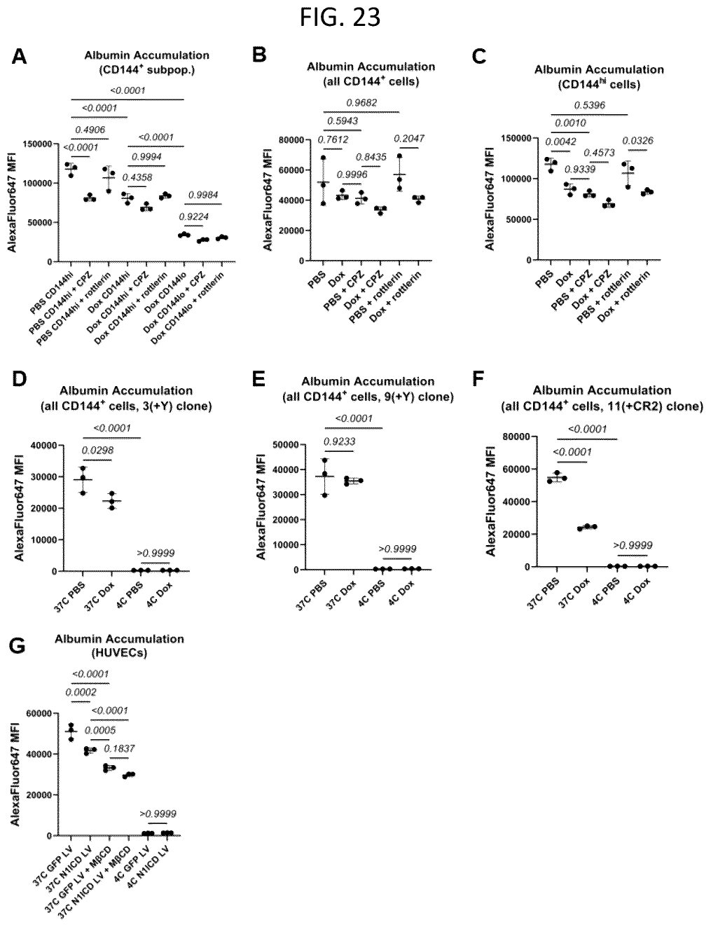

We also asked if Notch signaling activation caused this reduction in albumin accumulation because caveolae-mediated endocytosis was being reduced. We incubated both PBS- and Dox-treated cells with methyl-β-cyclodextrin (MβCD), an inhibitor of caveolae-mediated endocytosis; the dose of 3 mM MβCD was selected based on past literature⁵⁵. We found that the reduction in albumin accumulation achieved by blocking caveolae-mediated endocytosis alone (PBS+MβCD) was similar in magnitude to the reduction achieved via upregulation of Notch signaling (Dox). However, when comparing PBS+MβCD and Dox+MβCD, there was still a slight but significant reduction in albumin accumulation when Notch signaling activation was combined with blockade of caveolae-mediated endocytosis (FIG. 8D). This suggests that although Notch signaling primarily results in reduction of caveolae mediated endocytosis, other endocytic pathways are also reduced when this pathway is activated in our endothelial model. Therefore, we decided to investigate other common pathways reported in the literature for substrate trafficking across endothelial cells, including clathrin-mediated endocytosis and macropinocytosis⁵⁶. We inhibited clathrin-mediated endocytosis with chlorpromazine (CPZ) and macropinocytosis with rottlerin, as previously described⁸. We observed that albumin accumulation was reduced when PBS-treated cells were incubated with CPZ, but not with rottlerin (FIG. 23A-C), suggesting that, in addition to caveolae-mediated endocytosis, clathrin-mediated endocytosis is another possible mechanism for intracellular accumulation of albumin. We again observed a N1ICD overexpression dose-dependent reduction in albumin accumulation among the PBS- and Dox-treated subpopulations (FIG. 23A). We also observed recapitulation of the Dox/N1ICD overexpression mediated reduction in albumin accumulation, although the difference was only significant when examining the CD144hi subpopulations, likely due to significant variability in CD144 expression among the CD144+ Dox-treated cells (FIG. 23B-C). Thus, Notch signaling activation via N1ICD overexpression reduces caveolae- and clathrin-mediated endocytosis, but not macropinocytosis, and results in reduced accumulation of albumin in the gene-edited PB-TRE-N1ICD hPSC-EPCs in vitro.

Because these accumulation assays were performed in edited EPCs derived from the 10(+CR2) clone, we repeated the accumulation assay in the other 3 clones with various copy numbers of the Dox-inducible N1ICD overexpression cassette that were previously characterized, namely 3(+Y), 9(+Y), and 11 (+CR2). We found that fluorescent albumin accumulation was decreased in 2 of 3 of these clones (3[+Y] and 11[+CR2]), and the fold reduction in AlexaFluor 647 signal intensity correlated with the degree of N1ICD upregulation in each of these clones at the highest doxycycline dose relative their respective PBS controls (FIG. 23D-F, FIG. 17A). Finally, we investigated whether HUVECs transduced with N1ICD overexpressing lentivirus also demonstrated reduced albumin accumulation. We confirmed reduced albumin accumulation in these primary human endothelial cells (FIG. 23G); however, the degree of downregulation was less than when HUVECs were treated with caveolae inhibitor MβCD. This is unsurprising given that the level of N1ICD protein induced by lentiviral overexpression is much lower than with Dox-inducible overexpression in our edited cell line (FIG. 11E, FIG. 3D).

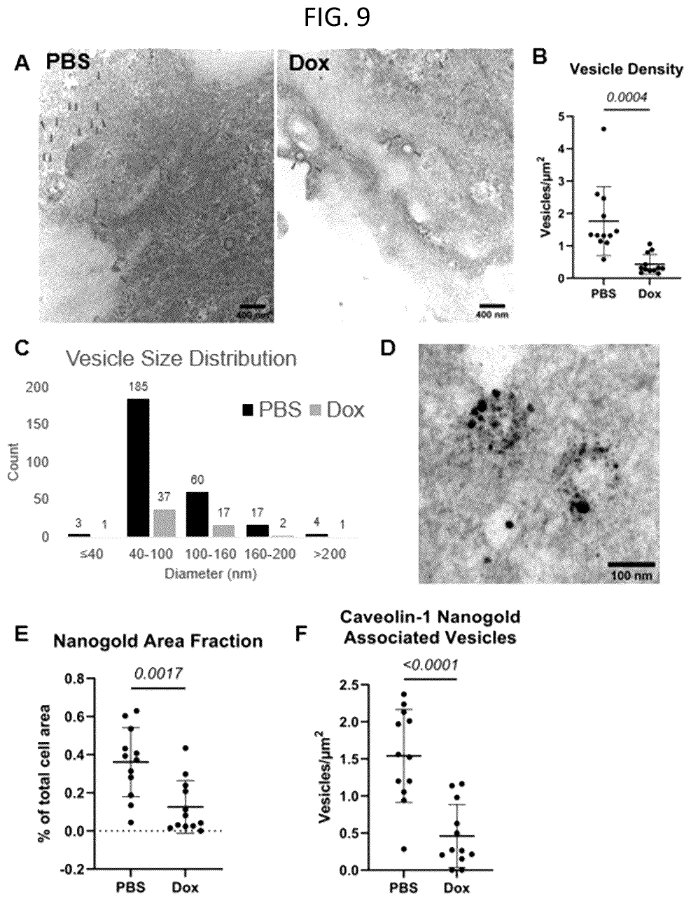

Finally, we visualized vesicles in doxycycline-treated 10(+CR2) PB-TRE-N1ICD hPSC-EPCs with transmission electron microscopy (TEM). We observed fewer vesicles per cell area in Dox-treated samples relative to PBS control (FIG. 9A-B). We measured the diameter for all identifiable vesicles in both conditions and found that the majority of vesicles were of the size range for caveolae (50-100 nm) and clathrin-coated vesicles (70-150 nm) described in the literature⁵⁶ (FIG. 9C). Upon treatment with doxycycline, there was a significant decrease in the number of vesicles between 40 and 160 nm in diameter (FIG. 9C). We also obtained images of cells immunolabeled for caveolin-1 with Nanogold-conjugated antibodies. In these images, caveolae appeared as circular regions with reduced electron density (less gray) compared to the surrounding cell area, with at least one (more frequently several) adjacent dark, electron-dense dot, representative of Nanogold particles (FIG. 9D). In Dox-treated cells, we observed a smaller percentage of the total cell area taken up by caveolin-1 associated Nanogold particles (FIG. 9E) and fewer caveolin-1 Nanogold-associated vesicles per cell area (FIG. 9F). These data indicate that not only does upregulation of Notch signaling via N1ICD overexpression reduce caveolin-1 expression, but it also reduces the average number of vesicles and the number of caveolae, specifically. Although our data suggest that there are fewer vesicles of similar size to clathrin-coated vesicles, we do not have direct evidence as with the caveolin-1-associated vesicles.

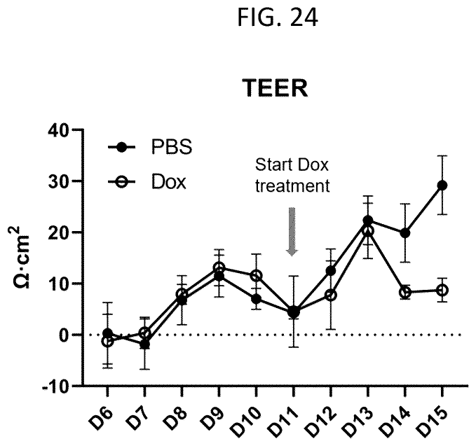

We also measured trans-endothelial electrical resistance (TEER), which estimates barrier tightness, in gene-edited PB-TRE-N1ICD hPSC-EPCs treated with or without doxycycline over 10 days in culture (FIG. 24). We observed that while initially, the temporal increase in TEER observed in the control cells was roughly matched by the Dox-treated cells, after more than 2 days of doxycycline treatment, TEER began to decline relative to the control. This is unsurprising as we observed transcript- and protein-level decreases in claudin-5 (FIG. 18) which contributes to barrier integrity at the BBB. Because average TEER in the Dox-treated edited hPSC-EPCs at the end of the culture time (D15) was less than 10 Ω·cm², we did not proceed with a fluorescent albumin transcytosis assay, as paracellular leakage of fluorescent substrate would complicate interpretation of substrate crossing by the transcellular pathway.

### DISCUSSION

Notch signaling is known to play several important roles in the development and maintenance of the vasculature²¹. Although limited evidence in the literature suggests that Notch signaling is involved in the induction of BBB properties in humans, most of our understanding about this pathway's contribution centers on the role of NOTCH3 in the attachment, differentiation, and survival of brain pericytes at the BBB²³. Signaling by Notch ligand D114 through endothelial cell-specific Notch1 receptor in adult mice maintained reduced transcytosis properties of the blood-retina barrier⁴. In developing mice, D114 knockdown resulted in increased brain albumin accumulation⁵⁷. DLL4 insufficiency reduced TEER and increased permeability in primary human BMECs, and there was increased NOTCH1 density at the RBPJ binding site within the CLDN5 promoter⁵⁷.

In this study, we demonstrated through a combination of protein-level characterization, bulk RNA-seq, and functional assays that Notch signaling activation (by overexpression of N1ICD) in hPSC-derived EPCs pre-treated with Wnt signaling activation leads to induction of additional BBB-like properties that were not previously achieved with activation of Wnt signaling alone. The main BBB property induced by N1ICD overexpression was reduced vesicular endocytosis. We were not able to demonstrate reduced transcytosis in vitro due to low paracellular barrier resistance. We were able to recapitulate the phenomenon of reduced caveolin-1 and reduced vesicular endocytosis in primary endothelial cells (e.g., HUVECs) which are more terminally differentiated than our hPSC-derived EPCs, suggesting that Notch signaling-mediated reduction in vesicular trafficking is not limited to progenitor-like endothelial cells. This is in contrast to a previous study⁸ which demonstrated that developmental plasticity of endothelial cells is a key determinant in their response to BBB property-inducing signals such as canonical Wnt signaling activation. The degree of fluorescent substrate accumulation observed in HUVECs due to N1ICD overexpression was less than observed in the gene-edited hPSC-EPCs treated with doxycycline, but this is likely due to the reduced gene dosage of N1ICD overexpression achieved by the lentiviral overexpression system compared to the cell line with inducible N1ICD upregulation.

However, we identified several limitations to artificial activation of Notch signaling in our human-derived endothelial model system. We observed reduced transcript and protein-level expression of claudin-5, as well as reduced transcript level expression of occludin and lower TEER. Thus, in our model system, sufficiently high gene dosage of N1ICD overexpression can reduce tight junction expression and paracellular barrier integrity. This is in contrast to another aforementioned study which demonstrated signaling through NOTCH1 in primary human BMECs is important for maintenance of claudin-5 expression⁵⁷; this potentially draws a distinction between the role of Notch signaling in BBB property induction vs. maintenance. Further tuning of the degree of Notch signaling activation with different clones of the Dox-inducible N1ICD overexpressing hPSC-line and modulation of other BBB-relevant signaling pathways, such as TGF-β inhibtion⁵⁸, may ameliorate the claudin-5 reduction.

Our study also demonstrated reduced expression of endothelial markers CD31 (PECAM1) and CD144 (VE-cadherin) proportional to the gene dosage of N1ICD overexpression. However, the comparisons of mouse single cell transcriptomes of brain capillary endothelial and peripheral organ (e.g., lung) endothelial cells suggest that BMECs have ˜2-fold and ˜6-fold lower expression of Pecam1 and Cdh5 (encoding VE-cadherin), respectively, relative to peripheral endothelial cells^{53,59}. Additionally, in vivo human brain endothelial cells have increased expression of FN125, encoding fibronectin, which in the context of many other endothelial cell types is a marker of endothelial to mesenchymal transition⁵². These observations indicate that in the process of acquiring their unique phenotype, they become a distinctly different type of endothelial cell compared to those in capillary beds of peripheral organs.

A natural question that arises upon confirmation of the effects of Notch signaling in vitro is the potential origin of those signals in vivo. Some have suggested Notch signaling in brain microvascular endothelial cells in vivo may occur through juxtacrine signaling from mural²³ cells via unknown Notch ligands. In a recent scRNA-seq analysis of the human fetal brain vasculature⁴⁶ Crouch and colleagues show that early in development (15 gestational weeks, or GWs), brain endothelial cells of various subtypes, including arterial, capillary, venous, tip, and mitotic endothelial cells, primarily express NOTCH1 and NOTCH4 receptor genes-arterial cells have the highest expression of NOTCH1. In contrast, mural cells including classical pericytes, smooth muscle cells (SMCs), and mitotic mural cells mainly express NOTCH3, which has previously been reported^{42,60,61}. Most interestingly, the primary cell types expressing Notch ligands genes including DLL4, JAG1, and JAG2 during human development are endothelial cells, particularly arterial and tip cells, suggesting that much of the BBB-inductive signaling associated with the Notch pathway may be a juxtracrine process between different endothelial subtypes⁴⁶. Later in development (23 GWs), the primary endothelial Notch receptor is NOTCH4, and arterial and tip endothelial cells mainly express the ligand DLL4, whereas SMCs in the ganglionic eminence/subventricular zone express JAG1, which can also potentially communicate with endothelial Notch receptors⁴⁶. Induction of BBB-related genes coincident with upregulation in Notch-related gene expression has also been observed in vascular organoids co-cultured with neural organoids⁶². The authors observed spontaneous emergence of mural cells as well as several endothelial subtypes in these assembloids mirroring those found at the brain arteriovenous tree^{46,47,63-65}, contributing to activation of Notch signaling.

Another study⁶⁶ demonstrated that natural aging in mice resulted in increased permeability of the brain vasculature to plasma proteins which correlated with decreased expression of genes associated with receptor-mediated transcytosis and clathrin-mediated endocytosis, increased expression of genes associated with caveolae-mediated transcytosis, and decreased expression of MFSD2A protein. However, there was no overt suggestion that these phenomena correlated with decreased endothelial cell-intrinsic Notch signaling.

An important contribution of this work is the discovery that Notch signaling activation induces upregulation of MFSD2A in a human-derived endothelial in vitro model. Previously, only Wnt⁵ and PTEN/AKT⁶⁷ signaling were reported to mediate increased MFSD2A expression at the BBB. It is important to note that while earlier evidence suggested that MFSD2A is required for the maintenance of barrier integrity⁷, several subsequent studies⁶⁸⁻⁷⁰ have suggested the absence of a causal link between increased MFSD2A expression at the BBB and reduced vesicular transcytosis. In Mfsd2a knockout mice, there was no observed increase in leakiness of fluorescent tracers of several sizes across the blood-retina⁶⁸ or blood-brain barriers⁶⁸⁻⁷⁰. The present study reinforces a strong association between increased Notch signaling, MFSD2A upregulation, caveolin-1 downregulation, and reduced caveolae-mediated endocytosis.

Furthermore, the cell line with doxycycline-inducible N1ICD overexpression is versatile in that it can be differentiated into other cell types which rely on Notch signaling activation for induction of key functional properties, such as neural stem cells⁷¹ and T cells⁷². Based on unpublished observations from our own lab, overexpression of the Notch receptor intracellular domain is a much more potent activator of downstream Notch-related gene expression than Notch ligand-presenting cells or Notch ligand-conjugated beads.

### CONCLUSIONS

In this study, we demonstrated for the first time in a human-derived in vitro endothelial model system that Notch signaling plays an important role in the induction of certain BBB properties. These include reduced expression of caveolin-1 and PLVAP, and increased expression of MFSD2A, which also correlates with reduced vesicle-based endocytosis of substrates and fewer caveolar vesicles. Furthermore, we demonstrated that Notch signaling activation can also increase expression of BBB-enriched glucose transporter GLUT-1, as well as a handful of other BBB-enriched selective nutrient transporters. We also observed that despite the reduction in expression of endothelial markers CD31 and CD144 (which may be more reflective of in vivo reality), endothelial cells subjected to Notch signaling activation via overexpression of N1ICD maintain their endothelial identity and do not take on characteristics of other cell types such as smooth or skeletal muscle cells, epithelial cells, or fibroblasts. However, some limitations of this protocol include reduced expression of claudin-5 and occludin, and little to no induction of efflux transporter P-glycoprotein or other drug efflux pump transcripts. Nonetheless, our findings contribute to the understanding of the complex interplay of various cell signals that are important for induction of BBB properties and are therefore a step towards generation of improved human-derived in vitro BBB models.

### REFERENCES

ADDIN Mendeley Bibliography CSL_BIBLIOGRAPHY 1. Obermeier, B., Daneman, R. & Ransohoff,
R. M. Development, maintenance and disruption of the blood-brain barrier. Nat. Med. 19, 1584-1596 (2013).
2. Zhao, Z., Nelson, A. R., Betsholtz, C. & Zlokovic, B. V. Establishment and Dysfunction of the Blood-Brain Barrier. Cell 163, 1064-1078 (2015).
3. Workman, M. J. & Svendsen, C. N. Recent advances in human iPSC-derived models of the blood-brain barrier. Fluids Barriers CNS 17, 1-10(2020).
4. Yang, J. M. et al. DLL4 suppresses transcytosis for arterial blood-retinal barrier homeostasis. Circ. Res. 126, 767-783(2020).
5. Wang, Z. et al. Wnt signaling activates MFSD2A to suppress vascular endothelial transcytosis and maintain blood-retinal barrier. Sci. Adv. 6, (2020).
6. Andreone, B. J. et al. Blood-Brain Barrier Permeability Is Regulated by Lipid Transport-Dependent Suppression of Caveolae-Mediated Transcytosis. Neuron 94, 581-594 (2017).
7. Ben-Zvi, A. et al. Mfsd2a is critical for the formation and function of the blood-brain barrier. Nature 509, 507-511 (2014).
8. Gastfriend, B. D. et al. Wnt signaling mediates acquisition of blood-brain barrier properties in naive endothelium derived from human pluripotent stem cells. Elife 10, 1-33 (2021).
9. Wilhelm, I., Nyúl-Tóth, Á., Suciu, M., Hermenean, A. & Krizbai, I. A. Heterogeneity of the blood-brain barrier. Tissue Barriers 4, 1-8 (2016).
10. Saunders, N. R., Liddelow, S. A. & Dziegielewska, K. M. Barrier mechanisms in the developing brain. Front. Pharmacol. 3, 1-18 (2012).
11. Berndt, P. et al. Tight junction proteins at the blood-brain barrier: far more than claudin-5. Cell. Mol. Life Sci. 76, 1987-2002 (2019).
12. Sohet, F. et al. LSR/angulin-1 is a tricellular tight junction protein involved in blood-brain barrier formation. J. Cell Biol. 208, 703-711 (2015).
13. Morris, M. E., Rodriguez-Cruz, V. & Felmlee, M. A. SLC and ABC Transporters: Expression, Localization, and Species Differences at the Blood-Brain and the Blood-Cerebrospinal Fluid Barriers. AAPS J. 19, 1317-1331 (2017).
14. Butterworth IV, J. F., Mackey, D. C. & Wasnick, J. D. in Morgan and Mikhail's Clinical Anesthesiology, 7e (McGraw-Hill Education, 2022). at <http://accessmedicine.mhmedical.com/content.aspx?aid=1190607015>
15. Pardridge, W. M. The Blood-Brain Barrier: Bottleneck in Brain Drug Development. NeuroRx 2, 3-14 (2005).
16. Engelhardt, B. & Liebner, S. Novel insights into the development and maintenance of the blood-brain barrier. Cell Tissue Res. 355, 687-699(2014).
17. Villabona-Rueda, A., Erice, C., Pardo, C. A. & Stins, M. F. The Evolving Concept of the Blood Brain Barrier (BBB): From a Single Static Barrier to a Heterogeneous and Dynamic Relay Center. Front. Cell. Neurosci. 13, 1-9(2019).
18. Daneman, R. et al. Wnt/β-catenin signaling is required for CNS, but not non-CNS, angiogenesis. Proc. Natl. Acad. Sci. U.S.A 106, 641-646 (2009).
19. Stenman, J. M. et al. Canonical Wnt Signaling Regulates Organ-Specific Assembly and Differentiation of CNS Vasculature. Science (80-.). 322, 1247-1250 (2008).
20. Wang, Y. et al. Beta-catenin signaling regulates barrier-specific gene expression in circumventricular organ and ocular vasculatures. Elife 8, 1-36 (2019).
21. Akil, A. et al. Notch Signaling in Vascular Endothelial Cells, Angiogenesis, and Tumor Progression: An Update and Prospective. Front. Cell Dev. Biol. 9, 1-16 (2021).
22. Mack, J. J. et al. NOTCH1 is a mechanosensor in adult arteries. Nat. Commun. 8, 1-18 (2017).
23. Winkler, E. A., Bell, R. D. & Zlokovic, B. V. Central nervous system pericytes in health and disease. Nat. Neurosci. 14, 1398-1405 (2011).
24 Li, F. et al. Endothelial Smad4 Maintains Cerebrovascular Integrity by Activating N-Cadherin through Cooperation with Notch. Dev. Cell 20, 291-302 (2011).
25. Gastfriend, B. D., Foreman, K. L., Katt, M. E., Palecek, S. P. & Shusta, E. V. Integrative analysis of the human brain mural cell transcriptome. J. Cereb. Blood Flow Metab. 41, 3052-3068 (2021).
26. Wong, B. H. & Silver, D. L. in Advances in Experimental Medicine and Biology 1276, 223-234 (2020).
27. Andreone, B. J. et al. Blood-Brain Barrier Permeability Is Regulated by Lipid Transport-Dependent Suppression of Caveolae-Mediated Transcytosis. Neuron 94, 581-594.e5 (2017).
28. Kuintzle, R., Santat, L. A. & Elowitz, M. B. Diversity in Notch ligand-receptor signaling interactions. Elife 1-80 (2023). at <https://doi.org/10.7554/eLife.91422.1https://en.wikipedia.org/wiki/Open_access>
29. Falo-Sanjuan, J. & Bray, S. J. Decoding the Notch signal. Dev. Growth Differ. 62, 4-14 (2020).
30. Zhou, B. et al. Notch signaling pathway: architecture, disease, and therapeutics. Signal Transduct. Target. Ther. 7, 1-33(2022).
31. Ayaz, F. & Osborne, B. A. Non-canonical Notch signaling in cancer and immunity. Front. Oncol. 4, 1-6 (2014).
32. Ye, Q. et al. Small molecule activation of NOTCH signaling inhibits acute myeloid leukemia. Sci. Rep. 6, 1-11 (2016).
33. Yu, X. M., Phan, T., Patel, P. N., Jaskula-Sztul, R. & Chen, H. Chrysin activates Notch1 signaling and suppresses tumor growth of anaplastic thyroid carcinoma in vitro and in vivo. Cancer 119, 774-781 (2013).
34 Patel, P. N., Yu, X. M., Jaskula-Sztul, R. & Chen, H. Hesperetin Activates the Notch1 Signaling Cascade, Causes Apoptosis, and Induces Cellular Differentiation in Anaplastic Thyroid Cancer. Ann. Surg. Oncol. 21, 497-504 (2014).
35. Guo, G. et al. N-methylhemeanthidine chloride, a novel Amaryllidaceae alkaloid, inhibits pancreatic cancer cell proliferation via down-regulating AKT activation. Toxicol. Appl. Pharmacol. 280, 475-483(2014).
36. Li, F., Ye, L., Lin, S. mei & Leung, L. K. Dietary flavones and flavonones display differential effects on aromatase (CYP19) transcription in the breast cancer cells MCF-7. Mol. Cell. Endocrinol. 344, 51-58 (2011).
37. Yang, Y., Wolfram, J., Shen, H., Fang, X. & Ferrari, M. Hesperetin: An inhibitor of the transforming growth factor-β (TGF-β) signaling pathway. Eur. J. Med. Chem. 58, 390-395 (2012).
38. Yu, L. et al. Adropin preserves the blood-brain barrier through a Notch1/Hes1 pathway after intracerebral hemorrhage in mice. J. Neurochem. 143, 750-760 (2017).
39. Cui, X. Y. et al. NB-3/Notch1 pathway via Deltex1 promotes neural progenitor cell differentiation into oligodendrocytes. J. Biol. Chem. 279, 25858-25865 (2004).
40. Lian, X. et al. Efficient Differentiation of Human Pluripotent Stem Cells to Endothelial Progenitors via Small-Molecule Activation of WNT Signaling. Stem Cell Reports 3, 1-13 (2014).
41. Nishihara, H. et al. Advancing human induced pluripotent stem cell-derived blood-brain barrier models for studying immune cell interactions. FASEB J. 34, 16693-16715 (2020).
42. Gastfriend, B. D., Snyder, M. E., Daneman, R., Palecek, S. P. & Shusta, E. V. Notch3 directs differentiation of brain mural cells from human pluripotent stem cell-derived neural crest. Sci. Adv. (2023).
43. Afgan, E. et al. The Galaxy platform for accessible, reproducible and collaborative biomedical analyses: 2016 update. Nucleic Acids Res. 44, W3-W10(2016).
44. Love, M. I., Huber, W. & Anders, S. Moderated estimation of fold change and dispersion for RNA-seq data with DESeq2. Genome Biol. 15, 1-21 (2014).
45. Lo, E. K. W. et al. Platform-agnostic CellNet enables cross-study analysis of cell fate engineering protocols. Stem Cell Reports 18, 1721-1742 (2023).
46. Crouch, E. E. et al. Ensembles of endothelial and mural cells promote angiogenesis in prenatal human brain. Cell 185, 3753-3769(2022).
47. Yang, A. C. et al. A human brain vascular atlas reveals diverse mediators of Alzheimer's risk. Nature 603, 885-892 (2022).
48. Stuart, T. et al. Comprehensive Integration of Single-Cell Data. Cell 177, 1888-1902 (2019).
49. Williams, T. M. & Lisanti, M. P. The caveolin proteins. Genome Biol. 5, 1-8 (2004).
50. Denzer, L., Muranyi, W., Schroten, H. & Schwerk, C. The role of PLVAP in endothelial cells. Cell Tissue Res. 392, 393-412 (2023).
51. Pandit, R. et al. Role for caveolin-mediated transcytosis in facilitating transport of large cargoes into the brain via ultrasound. J. Control. Release 327, 667-675 (2020).
52. Dejana, E., Hirschi, K. K. & Simons, M. The molecular basis of endothelial cell plasticity. Nat. Commun. 8, 1-11 (2017).
53. He, L. et al. Single-cell RNA sequencing of mouse brain and lung vascular and vessel-associated cell types. Sci. Data 5, 1-11 (2018).
54. Qian, T. et al. Directed differentiation of human pluripotent stem cells to blood-brain barrier endothelial cells. Sci. Adv. 3, 1-12 (2017).
55. Guo, S. et al. Selectivity of commonly used inhibitors of clathrin-mediated and caveolae-dependent endocytosis of G protein-coupled receptors. Biochim. Biophys. Acta-Biomembr. 1848, 2101-2110 (2015).
56. De Bock, M. et al. Into rather unexplored terrain-transcellular transport across the blood-brain barrier. Glia 64, 1097-1123(2016).
57. Ke, X. et al. Delta like 4 regulates cerebrovascular development and endothelial integrity via DLL4-NOTCH-CLDN5 pathway and is vulnerable to neonatal hyperoxia. J. Physiol. 10, 2265-2285 (2024).
58. Roudnicky, F. et al. Inducers of the endothelial cell barrier identified through chemogenomic screening in genome-edited hPSC-endothelial cells. Proc. Natl. Acad. Sci. U.S.A 117, 19854-19865 (2020).
59. Vanlandewijck, M. et al. A molecular atlas of cell types and zonation in the brain vasculature. Nature 554, 475-480 (2018).
60. Liu, H., Kennard, S. & Lilly, B. NOTCH3 expression is induced in mural cells through an autoregulatory loop that requires Endothelial-expressed JAGGED1. Circ. Res. 104, 466-475 (2009).
61. Liu, H., Zhang, W., Kennard, S., Caldwell, R. B. & Lilly, B. Notch3 is critical for proper angiogenesis and mural cell investment. Circ. Res. 107, 860-870 (2010).
62. Dao, L. et al. Modeling blood-brain barrier formation and cerebral cavernous malformations in human PSC-derived organoids. Cell Stem Cell 31, 1-16 (2024).
63. Garcia, F. J. et al. Single-cell dissection of the human brain vasculature. Nature 603, 893-899(2022).
64. Sun, N. et al. Single-nucleus multiregion transcriptomic analysis of brain vasculature in Alzheimer's disease. Nat. Neurosci. 26, 970-982 (2023).
65. Wälchli, T. et al. Single-cell atlas of the human brain vasculature across development, adulthood and disease. Nature 1-12 (2024). doi: 10.1038/s41586-024-07493-y
66 Yang, A. C. et al. Physiological blood-brain transport is impaired with age by a shift in transcytosis. Nature 583, 425-430 (2020).
67. Cui, Y. et al. Brain endothelial PTEN/AKT/NEDD4-2/MFSD2A axis regulates blood-brain barrier permeability. Cell Rep. 36, 109327 (2021).
68. Wong, B. H. et al. Mfsd2a is a transporter for the essential ω-3 fatty acid docosahexaenoic acid (DHA) in eye and is important for photoreceptor cell development. J. Biol. Chem. 291, 10501-10514 (2016).
69. Lobanova, E. S. et al. Disrupted blood-retina lysophosphatidylcholine transport impairs photoreceptor health but not visual signal transduction. J. Neurosci. 39, 9689-9701 (2019).
70. Mäe, M. A. et al. Single-Cell Analysis of Blood-Brain Barrier Response to Pericyte Loss. Circ. Res. 128, E46-E62 (2021).
71. Hitoshi, S. et al. Primitive neural stem cells from the mammalian epiblast differentiate to definitive neural stem cells under the control of Notch signaling. Genes Dev. 18, 1806-1811 (2004).
72. Brandstadter, J. D. & Maillard, I. Notch signalling in T cell homeostasis and differentiation. Open Biol. 9, (2019).
73. Porkoláb, G. et al. Synergistic induction of blood-brain barrier properties. Proc. Natl. Acad. Sci. 121, 1-12 (2024).

What is claimed:

1. A gene-edited pluripotent stem cell that comprises a transgene comprising a polynucleotide encoding a Notch1 receptor intracellular domain (N1ICD) operably linked to an inducible promoter.

2. The pluripotent stem cell of claim 1, wherein the inducible promoter is a doxycycline-inducible promoter.

3. The pluripotent stem cell of claim 1, wherein the pluripotent stem cell comprises 2-48 copies of the transgene.

4. A CD34+CD31+ endothelial progenitor cell differentiated from the pluripotent stem cell of claim 1.

5. A method for producing an endothelial cell with blood-brain barrier (BBB)-like transcytosis properties, the method comprising:
(a) culturing a CD34+CD31+ endothelial progenitor cell in a medium comprising a Wnt/β-catenin signaling activator; and
(b) inducing Notch signaling after 2-7 days of culturing.

6. The method of claim 5, wherein the endothelial cell produced by the method exhibits:
a) reduced expression of caveolin-1 relative to a non-BBB endothelial cell,
b) reduced expression of plasmalemma vesicle associated protein (PLVAP) relative to a non-BBB endothelial cell,
c) increased expression of GLUT-1 relative to a non-BBB endothelial cell,
d) increased expression of MFSD2A relative to a non-BBB endothelial cell,
e) increased expression of SLC1A4, SLC1A5, SLC7A5, SLC7A1, SLC7A2, SLC7A6, SLC5A6, and/or SLC2A3 relative to an endothelial cell produced by performing only step (a) of the method; and/or
f) reduced vesicular endocytosis relative to an endothelial cell produced by performing only step (a) of the method.

7. The method of claim 5, wherein the endothelial progenitor cell was differentiated from a pluripotent stem cell.

8. The method of claim 5, wherein the Wnt/β-catenin signaling activator is CHIR99021.

9. The method of claim 5, wherein Notch signaling is induced via overexpression of a N1ICD.

10. The method of claim 9, wherein overexpression of the N1ICD is achieved via transduction with a virus comprising a polynucleotide encoding the N1ICD.

11. The method of claim 9, wherein the endothelial progenitor cell is a CD34+CD31+ endothelial progenitor cell differentiated from a gene-edited pluripotent stem cell comprising a transgene comprising a polynucleotide encoding the N1ICD operably linked to an inducible promoter, and wherein overexpression of the N1ICD is achieved by contacting the endothelial progenitor cell with a reagent that induces expression from the inducible promoter.

12. The method of claim 11, wherein the inducible promoter is a doxycycline-inducible promoter, and wherein the reagent is doxycycline.

13. The method of claim 9, wherein the N1ICD comprises SEQ ID NO: 3.

14. The method of claim 5, wherein Notch signaling is induced for 3-6 days.

15. The method of claim 5, wherein the medium further comprises human endothelial serum-free medium (hESFM) supplemented with B-27 supplement and/or FGF2.

16. A population of endothelial cells with BBB-like transcytosis properties produced by the method of claim 5.

17. An in vitro BBB model comprising a confluent monolayer of the endothelial cells of claim 16 cultured on a surface, wherein the BBB model has BBB-like transcytosis properties.

18. The BBB model of claim 17, wherein the BBB model is an isogenic model.

19. The BBB model of claim 18, wherein the endothelial cells are derived from pluripotent stem cells obtained from a subject.

20. A method for using the BBB model of claim 17 to test the ability of a therapeutic agent to cross the BBB.
